SEQUENCE LISTING (225 sequences)

SEQ ID NO: 1 - protein, 5 aa, Artificial Sequence
  Description of Artificial Sequence: Synthetic peptide
TSINN

SEQ ID NO: 2 - protein, 7 aa, Artificial Sequence
  Description of Artificial Sequence: Synthetic peptide
IRSNERE

SEQ ID NO: 3 - protein, 14 aa, Artificial Sequence
  Description of Artificial Sequence: Synthetic peptide
CATTGDSGYSTLTF

SEQ ID NO: 4 - protein, 5 aa, Artificial Sequence
  Description of Artificial Sequence: Synthetic peptide
MNHEY

SEQ ID NO: 5 - protein, 6 aa, Artificial Sequence
  Description of Artificial Sequence: Synthetic peptide
SVGAGI

SEQ ID NO: 6 - protein, 13 aa, Artificial Sequence
  Description of Artificial Sequence: Synthetic peptide
CASTRQGGNEQFF

SEQ ID NO: 7 - protein, 5 aa, Artificial Sequence
  Description of Artificial Sequence: Synthetic peptide
TSINN

SEQ ID NO: 8 - protein, 7 aa, Artificial Sequence
  Description of Artificial Sequence: Synthetic peptide
IRSNERE

SEQ ID NO: 9 - protein, 14 aa, Artificial Sequence
  Description of Artificial Sequence: Synthetic peptide
CATEGDSGYSTLTF

SEQ ID NO: 10 - protein, 5 aa, Artificial Sequence
  Description of Artificial Sequence: Synthetic peptide
MNHEY

SEQ ID NO: 11 - protein, 6 aa, Artificial Sequence
  Description of Artificial Sequence: Synthetic peptide
SVGAGI

SEQ ID NO: 12 - protein, 14 aa, Artificial Sequence
  Description of Artificial Sequence: Synthetic peptide
CASSYQGGNYGYTF

SEQ ID NO: 13 - protein, 5 aa, Artificial Sequence
  Description of Artificial Sequence: Synthetic peptide
TSINN

SEQ ID NO: 14 - protein, 7 aa, Artificial Sequence
  Description of Artificial Sequence: Synthetic peptide
IRSNERE

SEQ ID NO: 15 - protein, 14 aa, Artificial Sequence
  Description of Artificial Sequence: Synthetic peptide
CATEGGSGYSTLTF

SEQ ID NO: 16 - protein, 5 aa, Artificial Sequence
  Description of Artificial Sequence: Synthetic peptide
MNHEY

SEQ ID NO: 17 - protein, 6 aa, Artificial Sequence
  Description of Artificial Sequence: Synthetic peptide
SVGAGI

SEQ ID NO: 18 - protein, 14 aa, Artificial Sequence
  Description of Artificial Sequence: Synthetic peptide
CASNTQGGHYGYTF

SEQ ID NO: 19 - protein, 5 aa, Artificial Sequence
  Description of Artificial Sequence: Synthetic peptide
TSINN

SEQ ID NO: 20 - protein, 7 aa, Artificial Sequence
  Description of Artificial Sequence: Synthetic peptide
IRSNERE

SEQ ID NO: 21 - protein, 14 aa, Artificial Sequence
  Description of Artificial Sequence: Synthetic peptide
CATQGDSGYSTLTF

SEQ ID NO: 22 - protein, 5 aa, Artificial Sequence
  Description of Artificial Sequence: Synthetic peptide
MNHEY

SEQ ID NO: 23 - protein, 6 aa, Artificial Sequence
  Description of Artificial Sequence: Synthetic peptide
SVGAGI

SEQ ID NO: 24 - protein, 15 aa, Artificial Sequence
  Description of Artificial Sequence: Synthetic peptide
CASSYQGGTRNGYTF

SEQ ID NO: 25 - protein, 5 aa, Artificial Sequence
  Description of Artificial Sequence: Synthetic peptide
TSINN

SEQ ID NO: 26 - protein, 7 aa, Artificial Sequence
  Description of Artificial Sequence: Synthetic peptide
IRSNERE

SEQ ID NO: 27 - protein, 14 aa, Artificial Sequence
  Description of Artificial Sequence: Synthetic peptide
CATVGNSGYSTLTF

SEQ ID NO: 28 - protein, 5 aa, Artificial Sequence
  Description of Artificial Sequence: Synthetic peptide
MNHEY

SEQ ID NO: 29 - protein, 6 aa, Artificial Sequence
  Description of Artificial Sequence: Synthetic peptide
SVGAGI

SEQ ID NO: 30 - protein, 14 aa, Artificial Sequence
  Description of Artificial Sequence: Synthetic peptide
CASSGQGGSDGYTF

SEQ ID NO: 31 - protein, 5 aa, Artificial Sequence
  Description of Artificial Sequence: Synthetic peptide
TSINN

SEQ ID NO: 32 - protein, 7 aa, Artificial Sequence
  Description of Artificial Sequence: Synthetic peptide
IRSNERE

SEQ ID NO: 33 - protein, 14 aa, Artificial Sequence
  Description of Artificial Sequence: Synthetic peptide
CATNGESGYSTLTF

SEQ ID NO: 34 - protein, 5 aa, Artificial Sequence
  Description of Artificial Sequence: Synthetic peptide
MNHEY

SEQ ID NO: 35 - protein, 6 aa, Artificial Sequence
  Description of Artificial Sequence: Synthetic peptide
SVGAGI

SEQ ID NO: 36 - protein, 14 aa, Artificial Sequence
  Description of Artificial Sequence: Synthetic peptide
CASSTQGGGDGYTF

SEQ ID NO: 37 - protein, 5 aa, Artificial Sequence
  Description of Artificial Sequence: Synthetic peptide
TSINN

SEQ ID NO: 38 - protein, 7 aa, Artificial Sequence
  Description of Artificial Sequence: Synthetic peptide
IRSNERE

SEQ ID NO: 39 - protein, 14 aa, Artificial Sequence
  Description of Artificial Sequence: Synthetic peptide
CAYEGNSGYSTLTF

SEQ ID NO: 40 - protein, 5 aa, Artificial Sequence
  Description of Artificial Sequence: Synthetic peptide
MNHEY

SEQ ID NO: 41 - protein, 6 aa, Artificial Sequence
  Description of Artificial Sequence: Synthetic peptide
SVGAGI

SEQ ID NO: 42 - protein, 14 aa, Artificial Sequence
  Description of Artificial Sequence: Synthetic peptide
CASSTQGGADGYTF

SEQ ID NO: 43 - protein, 5 aa, Artificial Sequence
  Description of Artificial Sequence: Synthetic peptide
TSINN

SEQ ID NO: 44 - protein, 7 aa, Artificial Sequence
  Description of Artificial Sequence: Synthetic peptide
IRSNERE

SEQ ID NO: 45 - protein, 14 aa, Artificial Sequence
  Description of Artificial Sequence: Synthetic peptide
CATEGDSGYSTLTF

SEQ ID NO: 46 - protein, 5 aa, Artificial Sequence
  Description of Artificial Sequence: Synthetic peptide
MNHEY

SEQ ID NO: 47 - protein, 6 aa, Artificial Sequence
  Description of Artificial Sequence: Synthetic peptide
SVGAGI

SEQ ID NO: 48 - protein, 14 aa, Artificial Sequence
  Description of Artificial Sequence: Synthetic peptide
CASRVQGGIEGYTF

SEQ ID NO: 49 - protein, 5 aa, Artificial Sequence
  Description of Artificial Sequence: Synthetic peptide
TSINN

SEQ ID NO: 50 - protein, 7 aa, Artificial Sequence
  Description of Artificial Sequence: Synthetic peptide
IRSNERE

SEQ ID NO: 51 - protein, 14 aa, Artificial Sequence
  Description of Artificial Sequence: Synthetic peptide
CATEGDSGYSTLTF

SEQ ID NO: 52 - protein, 5 aa, Artificial Sequence
  Description of Artificial Sequence: Synthetic peptide
MNHEY

SEQ ID NO: 53 - protein, 6 aa, Artificial Sequence
  Description of Artificial Sequence: Synthetic peptide
SVGAGI

SEQ ID NO: 54 - protein, 14 aa, Artificial Sequence
  Description of Artificial Sequence: Synthetic peptide
CASNQQGGGYGYTF

SEQ ID NO: 55 - protein, 5 aa, Artificial Sequence
  Description of Artificial Sequence: Synthetic peptide
TSINN

SEQ ID NO: 56 - protein, 7 aa, Artificial Sequence
  Description of Artificial Sequence: Synthetic peptide
IRSNERE

SEQ ID NO: 57 - protein, 14 aa, Artificial Sequence
  Description of Artificial Sequence: Synthetic peptide
CATEGDSGYSTLTF

SEQ ID NO: 58 - protein, 5 aa, Artificial Sequence
  Description of Artificial Sequence: Synthetic peptide
MNHEY

SEQ ID NO: 59 - protein, 6 aa, Artificial Sequence
  Description of Artificial Sequence: Synthetic peptide
SVGAGI

SEQ ID NO: 60 - protein, 14 aa, Artificial Sequence
  Description of Artificial Sequence: Synthetic peptide
CASRVQGGIEGYTF

SEQ ID NO: 61 - protein, 5 aa, Artificial Sequence
  Description of Artificial Sequence: Synthetic peptide
TSINN

SEQ ID NO: 62 - protein, 7 aa, Artificial Sequence
  Description of Artificial Sequence: Synthetic peptide
IRSNERE

SEQ ID NO: 63 - protein, 14 aa, Artificial Sequence
  Description of Artificial Sequence: Synthetic peptide
CATEGGSGYSTLTF

SEQ ID NO: 64 - protein, 5 aa, Artificial Sequence
  Description of Artificial Sequence: Synthetic peptide
MNHEY

SEQ ID NO: 65 - protein, 6 aa, Artificial Sequence
  Description of Artificial Sequence: Synthetic peptide
SVGAGI

SEQ ID NO: 66 - protein, 15 aa, Artificial Sequence
  Description of Artificial Sequence: Synthetic peptide
CASSFQGGGNYGYTF

SEQ ID NO: 67 - protein, 5 aa, Artificial Sequence
  Description of Artificial Sequence: Synthetic peptide
TSINN

SEQ ID NO: 68 - protein, 7 aa, Artificial Sequence
  Description of Artificial Sequence: Synthetic peptide
IRSNERE

SEQ ID NO: 69 - protein, 14 aa, Artificial Sequence
  Description of Artificial Sequence: Synthetic peptide
CATEGGSGYSTLTF

SEQ ID NO: 70 - protein, 5 aa, Artificial Sequence
  Description of Artificial Sequence: Synthetic peptide
MNHEY

SEQ ID NO: 71 - protein, 6 aa, Artificial Sequence
  Description of Artificial Sequence: Synthetic peptide
SVGAGI

SEQ ID NO: 72 - protein, 15 aa, Artificial Sequence
  Description of Artificial Sequence: Synthetic peptide
CASSSQGGWNYGYTF

SEQ ID NO: 73 - protein, 5 aa, Artificial Sequence
  Description of Artificial Sequence: Synthetic peptide
TSINN

SEQ ID NO: 74 - protein, 7 aa, Artificial Sequence
  Description of Artificial Sequence: Synthetic peptide
IRSNERE

SEQ ID NO: 75 - protein, 14 aa, Artificial Sequence
  Description of Artificial Sequence: Synthetic peptide
CAPLGEAGYSTLTF

SEQ ID NO: 76 - protein, 5 aa, Artificial Sequence
  Description of Artificial Sequence: Synthetic peptide
MNHEY

SEQ ID NO: 77 - protein, 6 aa, Artificial Sequence
  Description of Artificial Sequence: Synthetic peptide
SVGAGI

SEQ ID NO: 78 - protein, 14 aa, Artificial Sequence
  Description of Artificial Sequence: Synthetic peptide
CASSRQGSYNEQFF

SEQ ID NO: 79 - protein, 5 aa, Artificial Sequence
  Description of Artificial Sequence: Synthetic peptide
TSINN

SEQ ID NO: 80 - protein, 7 aa, Artificial Sequence
  Description of Artificial Sequence: Synthetic peptide
IRSNERE

SEQ ID NO: 81 - protein, 14 aa, Artificial Sequence
  Description of Artificial Sequence: Synthetic peptide
CATVGDSGYSTLTF

SEQ ID NO: 82 - protein, 5 aa, Artificial Sequence
  Description of Artificial Sequence: Synthetic peptide
MNHEY

SEQ ID NO: 83 - protein, 6 aa, Artificial Sequence
  Description of Artificial Sequence: Synthetic peptide
SVGAGI

SEQ ID NO: 84 - protein, 14 aa, Artificial Sequence
  Description of Artificial Sequence: Synthetic peptide
CASNPQGGSIGYTF

SEQ ID NO: 85 - protein, 5 aa, Artificial Sequence
  Description of Artificial Sequence: Synthetic peptide
TSINN

SEQ ID NO: 86 - protein, 7 aa, Artificial Sequence
  Description of Artificial Sequence: Synthetic peptide
IRSNERE

SEQ ID NO: 87 - protein, 14 aa, Artificial Sequence
  Description of Artificial Sequence: Synthetic peptide
CATEGGSGYSTLTF

SEQ ID NO: 88 - protein, 5 aa, Artificial Sequence
  Description of Artificial Sequence: Synthetic peptide
MNHEY

SEQ ID NO: 89 - protein, 6 aa, Artificial Sequence
  Description of Artificial Sequence: Synthetic peptide
SVGAGI

SEQ ID NO: 90 - protein, 13 aa, Artificial Sequence
  Description of Artificial Sequence: Synthetic peptide
CASTRQGGNEQFF

SEQ ID NO: 91 - protein, 5 aa, Artificial Sequence
  Description of Artificial Sequence: Synthetic peptide
TTLSN

SEQ ID NO: 92 - protein, 7 aa, Artificial Sequence
  Description of Artificial Sequence: Synthetic peptide
LVKSGEV

SEQ ID NO: 93 - protein, 9 aa, Artificial Sequence
  Description of Artificial Sequence: Synthetic peptide
CADPDGLIF

SEQ ID NO: 94 - protein, 5 aa, Artificial Sequence
  Description of Artificial Sequence: Synthetic peptide
SGHNS

SEQ ID NO: 95 - protein, 6 aa, Artificial Sequence
  Description of Artificial Sequence: Synthetic peptide
FNNNVP

SEQ ID NO: 96 - protein, 15 aa, Artificial Sequence
  Description of Artificial Sequence: Synthetic peptide
CASSLGAGAGYGYTF

SEQ ID NO: 97 - protein, 5 aa, Artificial Sequence
  Description of Artificial Sequence: Synthetic peptide
DSVNN

SEQ ID NO: 98 - protein, 5 aa, Artificial Sequence
  Description of Artificial Sequence: Synthetic peptide
IPSGT

SEQ ID NO: 99 - protein, 11 aa, Artificial Sequence
  Description of Artificial Sequence: Synthetic peptide
CAVDGGNKLVF

SEQ ID NO: 100 - protein, 5 aa, Artificial Sequence
  Description of Artificial Sequence: Synthetic peptide
SGHNS

SEQ ID NO: 101 - protein, 6 aa, Artificial Sequence
  Description of Artificial Sequence: Synthetic peptide
FNNNVP

SEQ ID NO: 102 - protein, 15 aa, Artificial Sequence
  Description of Artificial Sequence: Synthetic peptide
CASSLGAGSGWEQYF

SEQ ID NO: 103 - protein, 6 aa, Artificial Sequence
  Description of Artificial Sequence: Synthetic peptide
NYSPAY

SEQ ID NO: 104 - protein, 7 aa, Artificial Sequence
  Description of Artificial Sequence: Synthetic peptide
IRENEKE

SEQ ID NO: 105 - protein, 9 aa, Artificial Sequence
  Description of Artificial Sequence: Synthetic peptide
CLRNTPLVF

SEQ ID NO: 106 - protein, 5 aa, Artificial Sequence
  Description of Artificial Sequence: Synthetic peptide
SGHAT

SEQ ID NO: 107 - protein, 6 aa, Artificial Sequence
  Description of Artificial Sequence: Synthetic peptide
FQNNGV

SEQ ID NO: 108 - protein, 13 aa, Artificial Sequence
  Description of Artificial Sequence: Synthetic peptide
CASSLVPEETQYF

SEQ ID NO: 109 - protein, 6 aa, Artificial Sequence
  Description of Artificial Sequence: Synthetic peptide
NSASQS

SEQ ID NO: 110 - protein, 6 aa, Artificial Sequence
  Description of Artificial Sequence: Synthetic peptide
VYSSGN

SEQ ID NO: 111 - protein, 13 aa, Artificial Sequence
  Description of Artificial Sequence: Synthetic peptide
CVVRGYNFNKFYF

SEQ ID NO: 112 - protein, 6 aa, Artificial Sequence
  Description of Artificial Sequence: Synthetic peptide
DFQATT

SEQ ID NO: 113 - protein, 7 aa, Artificial Sequence
  Description of Artificial Sequence: Synthetic peptide
SNEGSKA

SEQ ID NO: 114 - protein, 18 aa, Artificial Sequence
  Description of Artificial Sequence: Synthetic peptide
CSARGPGEREANTDTQYF

SEQ ID NO: 115 - protein, 6 aa, Artificial Sequence
  Description of Artificial Sequence: Synthetic peptide
NSASQS

SEQ ID NO: 116 - protein, 6 aa, Artificial Sequence
  Description of Artificial Sequence: Synthetic peptide
VYSSGN

SEQ ID NO: 117 - protein, 13 aa, Artificial Sequence
  Description of Artificial Sequence: Synthetic peptide
CVVRGYNFNKFYF

SEQ ID NO: 118 - protein, 6 aa, Artificial Sequence
  Description of Artificial Sequence: Synthetic peptide
DFQATT

SEQ ID NO: 119 - protein, 7 aa, Artificial Sequence
  Description of Artificial Sequence: Synthetic peptide
SNEGSKA

SEQ ID NO: 120 - protein, 18 aa, Artificial Sequence
  Description of Artificial Sequence: Synthetic peptide
CSARGPRITDSSTDTQYF

SEQ ID NO: 121 - protein, 5 aa, Artificial Sequence
  Description of Artificial Sequence: Synthetic peptide
TTLSN

SEQ ID NO: 122 - protein, 7 aa, Artificial Sequence
  Description of Artificial Sequence: Synthetic peptide
LVKSGEV

SEQ ID NO: 123 - protein, 12 aa, Artificial Sequence
  Description of Artificial Sequence: Synthetic peptide
CARFSDGQKLLF

SEQ ID NO: 124 - protein, 5 aa, Artificial Sequence
  Description of Artificial Sequence: Synthetic peptide
MNHNY

SEQ ID NO: 125 - protein, 6 aa, Artificial Sequence
  Description of Artificial Sequence: Synthetic peptide
SVGAGI

SEQ ID NO: 126 - protein, 16 aa, Artificial Sequence
  Description of Artificial Sequence: Synthetic peptide
CASSCRGRGDCTEAFF

SEQ ID NO: 127 - protein, 6 aa, Artificial Sequence
  Description of Artificial Sequence: Synthetic peptide
NSAFQY

SEQ ID NO: 128 - protein, 6 aa, Artificial Sequence
  Description of Artificial Sequence: Synthetic peptide
TYSSGN

SEQ ID NO: 129 - protein, 12 aa, Artificial Sequence
  Description of Artificial Sequence: Synthetic peptide
CAMSAWASKIIF

SEQ ID NO: 130 - protein, 5 aa, Artificial Sequence
  Description of Artificial Sequence: Synthetic peptide
KGHDR

SEQ ID NO: 131 - protein, 6 aa, Artificial Sequence
  Description of Artificial Sequence: Synthetic peptide
SFDVKD

SEQ ID NO: 132 - protein, 16 aa, Artificial Sequence
  Description of Artificial Sequence: Synthetic peptide
CATSEKGTGRQETQYF

SEQ ID NO: 133 - protein, 5 aa, Artificial Sequence
  Description of Artificial Sequence: Synthetic peptide
TTLSN

SEQ ID NO: 134 - protein, 7 aa, Artificial Sequence
  Description of Artificial Sequence: Synthetic peptide
LVKSGEV

SEQ ID NO: 135 - protein, 12 aa, Artificial Sequence
  Description of Artificial Sequence: Synthetic peptide
CARFSDGQKLLF

SEQ ID NO: 136 - protein, 5 aa, Artificial Sequence
  Description of Artificial Sequence: Synthetic peptide
MNHNY

SEQ ID NO: 137 - protein, 6 aa, Artificial Sequence
  Description of Artificial Sequence: Synthetic peptide
SVGAGI

SEQ ID NO: 138 - protein, 15 aa, Artificial Sequence
  Description of Artificial Sequence: Synthetic peptide
CASSNRGGVPDTQYF

SEQ ID NO: 139 - protein, 9 aa, Human gammaherpesvirus 4
FLYALALLL

SEQ ID NO: 140 - protein, 10 aa, Human gammaherpesvirus 4
SSCSSCPLSK

SEQ ID NO: 141 - protein, 9 aa, Human gammaherpesvirus 4
IYVLVMLVL

SEQ ID NO: 142 - protein, 9 aa, Human gammaherpesvirus 4
TYGPVFMCL

SEQ ID NO: 143 - protein, 6 aa, Artificial Sequence
  Description of Artificial Sequence: Synthetic peptide
DSAIYN

SEQ ID NO: 144 - protein, 7 aa, Artificial Sequence
  Description of Artificial Sequence: Synthetic peptide
IQSSQRE

SEQ ID NO: 145 - protein, 13 aa, Artificial Sequence
  Description of Artificial Sequence: Synthetic peptide
CAVLMDSNYQLIW

SEQ ID NO: 146 - protein, 5 aa, Artificial Sequence
  Description of Artificial Sequence: Synthetic peptide
WSHSY

SEQ ID NO: 147 - protein, 6 aa, Artificial Sequence
  Description of Artificial Sequence: Synthetic peptide
SAAADI

SEQ ID NO: 148 - protein, 14 aa, Artificial Sequence
  Description of Artificial Sequence: Synthetic peptide
CASSEDGMNTEAFF

SEQ ID NO: 149 - protein, 5 aa, Artificial Sequence
  Description of Artificial Sequence: Synthetic peptide
DSVNN

SEQ ID NO: 150 - protein, 5 aa, Artificial Sequence
  Description of Artificial Sequence: Synthetic peptide
IPSGT

SEQ ID NO: 151 - protein, 11 aa, Artificial Sequence
  Description of Artificial Sequence: Synthetic peptide
CAVLNNNDMRF

SEQ ID NO: 152 - protein, 5 aa, Artificial Sequence
  Description of Artificial Sequence: Synthetic peptide
MGHRA

SEQ ID NO: 153 - protein, 6 aa, Artificial Sequence
  Description of Artificial Sequence: Synthetic peptide
YSYEKL

SEQ ID NO: 154 - protein, 13 aa, Artificial Sequence
  Description of Artificial Sequence: Synthetic peptide
CASSQGRWYEQYF

SEQ ID NO: 155 - protein, 7 aa, Artificial Sequence
  Description of Artificial Sequence: Synthetic peptide
NIATNDY

SEQ ID NO: 156 - protein, 5 aa, Artificial Sequence
  Description of Artificial Sequence: Synthetic peptide
GYKTK

SEQ ID NO: 157 - protein, 13 aa, Artificial Sequence
  Description of Artificial Sequence: Synthetic peptide
CLVGDQGYSTLTF

SEQ ID NO: 158 - protein, 5 aa, Artificial Sequence
  Description of Artificial Sequence: Synthetic peptide
SGHAT

SEQ ID NO: 159 - protein, 6 aa, Artificial Sequence
  Description of Artificial Sequence: Synthetic peptide
FQNNGV

SEQ ID NO: 160 - protein, 14 aa, Artificial Sequence
  Description of Artificial Sequence: Synthetic peptide
CASSFKLVEETQYF

SEQ ID NO: 161 - protein, 6 aa, Artificial Sequence
  Description of Artificial Sequence: Synthetic peptide
DSAIYN

SEQ ID NO: 162 - protein, 7 aa, Artificial Sequence
  Description of Artificial Sequence: Synthetic peptide
IQSSQRE

SEQ ID NO: 163 - protein, 12 aa, Artificial Sequence
  Description of Artificial Sequence: Synthetic peptide
CGGLGFGNVLHC

SEQ ID NO: 164 - protein, 6 aa, Artificial Sequence
  Description of Artificial Sequence: Synthetic peptide
GTSNPN

SEQ ID NO: 165 - protein, 5 aa, Artificial Sequence
  Description of Artificial Sequence: Synthetic peptide
SVGIG

SEQ ID NO: 166 - protein, 14 aa, Artificial Sequence
  Description of Artificial Sequence: Synthetic peptide
CAWSSLASSYEQYF

SEQ ID NO: 167 - protein, 9 aa, Human gammaherpesvirus 4
CLGGLLTMV

SEQ ID NO: 168 - protein, 9 aa, Artificial Sequence
  Description of Artificial Sequence: Synthetic peptide
SLGGLLTMV

SEQ ID NO: 169 - protein, 497 aa, Human gammaherpesvirus 4
MGSLEMVPMGAGPPSPGGDPDGYDGGNNSQYPSASGSSGNTPTPPNDEERESNEEPPPPYEDPYWGNGDRHSDYQPLGTQDQSLYLGLQHDGNDGLPPPPYSPRDDSSQHIYEEAGRGSMNPVCLPVIVAPYLFWLAAIAASCFTASVSTVVTATGLALSLLLLAAVASSYAAAQRKLLTPVTVLTAVVTFFAICLTWRIEDPPFNSLLFALLAAAGGLQGIYVLVMLVLLILAYRRRWRRLTVCGGIMFLACVLVLIVDAVLQLSPLLGAVTVVSMTLLLLAFVLWLSSPGGLGTLGAALLTLAAALALLASLILGTLNLTTMFLLMLLWTLVVLLICSSCSSCPLSKILLARLFLYALALLLLASALIAGGSILQTNFKSLSSTEFIPNLFCMLLLIVAGILFILAILTEWGSGNRTYGPVFMCLGGLLTMVAGAVWLTVMSNTLLSAWILTAGFLIFLIGFALFGVIRCCRYCCYYCLTLESEERPPTPYRNTV

SEQ ID NO: 170 - protein, 132 aa, Artificial Sequence
  Description of Artificial Sequence: Synthetic polypeptide
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATTGDSGYSTLTFGKGTMLLVSP

SEQ ID NO: 171 - protein, 131 aa, Artificial Sequence
  Description of Artificial Sequence: Synthetic polypeptide
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASTRQGGNEQFFGPGTRLTVL

SEQ ID NO: 172 - protein, 132 aa, Artificial Sequence
  Description of Artificial Sequence: Synthetic polypeptide
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATEGDSGYSTLTFGKGTMLLVSP

SEQ ID NO: 173 - protein, 132 aa, Artificial Sequence
  Description of Artificial Sequence: Synthetic polypeptide
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYQGGNYGYTFGSGTRLTVV

SEQ ID NO: 174 - protein, 132 aa, Artificial Sequence
  Description of Artificial Sequence: Synthetic polypeptide
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATEGGSGYSTLTFGKGTMLLVSP

SEQ ID NO: 175 - protein, 132 aa, Artificial Sequence
  Description of Artificial Sequence: Synthetic polypeptide
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASNTQGGHYGYTFGSGTRLTVV

SEQ ID NO: 176 - protein, 132 aa, Artificial Sequence
  Description of Artificial Sequence: Synthetic polypeptide
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATQGDSGYSTLTFGKGTMLLVSP

SEQ ID NO: 177 - protein, 133 aa, Artificial Sequence
  Description of Artificial Sequence: Synthetic polypeptide
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYQGGTRNGYTFGSGTRLTVV

SEQ ID NO: 178 - protein, 132 aa, Artificial Sequence
  Description of Artificial Sequence: Synthetic polypeptide
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATVGNSGYSTLTFGKGTMLLVSP

SEQ ID NO: 179 - protein, 132 aa, Artificial Sequence
  Description of Artificial Sequence: Synthetic polypeptide
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSGQGGSDGYTFGSGTRLTVV

SEQ ID NO: 180 - protein, 132 aa, Artificial Sequence
  Description of Artificial Sequence: Synthetic polypeptide
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATNGESGYSTLTFGKGTMLLVSP

SEQ ID NO: 181 - protein, 132 aa, Artificial Sequence
  Description of Artificial Sequence: Synthetic polypeptide
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSTQGGGDGYTFGSGTRLTVV

SEQ ID NO: 182 - protein, 132 aa, Artificial Sequence
  Description of Artificial Sequence: Synthetic polypeptide
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCAYEGNSGYSTLTFGKGTMLLVSP

SEQ ID NO: 183 - protein, 132 aa, Artificial Sequence
  Description of Artificial Sequence: Synthetic polypeptide
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSTQGGADGYTFGSGTRLTVV

SEQ ID NO: 184 - protein, 132 aa, Artificial Sequence; identical to SEQ ID NO: 172
  Description of Artificial Sequence: Synthetic polypeptide

SEQ ID NO: 185 - protein, 132 aa, Artificial Sequence
  Description of Artificial Sequence: Synthetic polypeptide
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRVQGGIEGYTFGSGTRLTVV

SEQ ID NO: 186 - protein, 132 aa, Artificial Sequence; identical to SEQ ID NO: 172
  Description of Artificial Sequence: Synthetic polypeptide

SEQ ID NO: 187 - protein, 132 aa, Artificial Sequence
  Description of Artificial Sequence: Synthetic polypeptide
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASNQQGGGYGYTFGSGTRLTVV

SEQ ID NO: 188 - protein, 132 aa, Artificial Sequence; identical to SEQ ID NO: 172
  Description of Artificial Sequence: Synthetic polypeptide

SEQ ID NO: 189 - protein, 132 aa, Artificial Sequence; identical to SEQ ID NO: 185
  Description of Artificial Sequence: Synthetic polypeptide

SEQ ID NO: 190 - protein, 132 aa, Artificial Sequence; identical to SEQ ID NO: 174
  Description of Artificial Sequence: Synthetic polypeptide

SEQ ID NO: 191 - protein, 133 aa, Artificial Sequence
  Description of Artificial Sequence: Synthetic polypeptide
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSFQGGGNYGYTFGSGTRLTVV

SEQ ID NO: 192 - protein, 132 aa, Artificial Sequence; identical to SEQ ID NO: 174
  Description of Artificial Sequence: Synthetic polypeptide

SEQ ID NO: 193 - protein, 133 aa, Artificial Sequence
  Description of Artificial Sequence: Synthetic polypeptide
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSSQGGWNYGYTFGSGTRLTVV

SEQ ID NO: 194 - protein, 132 aa, Artificial Sequence
  Description of Artificial Sequence: Synthetic polypeptide
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCAPLGEAGYSTLTFGKGTMLLVSP

SEQ ID NO: 195 - protein, 132 aa, Artificial Sequence
  Description of Artificial Sequence: Synthetic polypeptide
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSRQGSYNEQFFGPGTRLTVL

SEQ ID NO: 196 - protein, 132 aa, Artificial Sequence
  Description of Artificial Sequence: Synthetic polypeptide
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATVGDSGYSTLTFGKGTMLLVSP

SEQ ID NO: 197 - protein, 132 aa, Artificial Sequence
  Description of Artificial Sequence: Synthetic polypeptide
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASNPQGGSIGYTFGSGTRLTVV

SEQ ID NO: 198 - protein, 132 aa, Artificial Sequence; identical to SEQ ID NO: 174
  Description of Artificial Sequence: Synthetic polypeptide

SEQ ID NO: 199 - protein, 131 aa, Artificial Sequence; identical to SEQ ID NO: 171
  Description of Artificial Sequence: Synthetic polypeptide

SEQ ID NO: 200 - protein, 131 aa, Artificial Sequence
  Description of Artificial Sequence: Synthetic polypeptide
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVLMDSNYQLIWGAGTKLIIKP

SEQ ID NO: 201 - protein, 132 aa, Artificial Sequence
  Description of Artificial Sequence: Synthetic polypeptide
MGTRLFFYVALCLLWAGHRDAGITQSPRYKITETGRQVTLMCHQTWSHSYMFWYRQDLGHGLRLIYYSAAADITDKGEVPDGYVVSRSKTENFPLTLESATRSQTSVYFCASSEDGMNTEAFFGQGTRLTVV

SEQ ID NO: 202 - protein, 125 aa, Artificial Sequence
  Description of Artificial Sequence: Synthetic polypeptide
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCADPDGLIFGQGTELSVKP

SEQ ID NO: 203 - protein, 134 aa, Artificial Sequence
  Description of Artificial Sequence: Synthetic polypeptide
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSLGAGAGYGYTFGSGTRLTVV

SEQ ID NO: 204 - protein, 127 aa, Artificial Sequence
  Description of Artificial Sequence: Synthetic polypeptide
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVDGGNKLVFGAGTILRVKS

SEQ ID NO: 205 - protein, 134 aa, Artificial Sequence
  Description of Artificial Sequence: Synthetic polypeptide
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSLGAGSGWEQYFGPGTRLTVT

SEQ ID NO: 206 - protein, 147 aa, Artificial Sequence
  Description of Artificial Sequence: Synthetic polypeptide
MAFWLRRLGLHFRPHLGRRMESFLGGVLLILWLQVDWVKSQKIEQNSEALNIQEGKTATLTCNYTNYSPAYLQWYRQDPGRGPVFLLLIRENEKEKRKERLKVTFDTTLKQSLFHITASQPADSATYLCLRNTPLVFGKGTRLSVIA

SEQ ID NO: 207 - protein, 132 aa, Artificial Sequence
  Description of Artificial Sequence: Synthetic polypeptide
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLVPEETQYFGPGTRLLVL

SEQ ID NO: 208 - protein, 127 aa, Artificial Sequence
  Description of Artificial Sequence: Synthetic polypeptide
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVLNNNDMRFGAGTRLTVKP

SEQ ID NO: 209 - protein, 131 aa, Artificial Sequence
  Description of Artificial Sequence: Synthetic polypeptide
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQGRWYEQYFGPGTRLTVT

SEQ ID NO: 210 - protein, 127 aa, Artificial Sequence
  Description of Artificial Sequence: Synthetic polypeptide
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGDQGYSTLTFGKGTMLLVSP

SEQ ID NO: 211 - protein, 133 aa, Artificial Sequence
  Description of Artificial Sequence: Synthetic polypeptide
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSFKLVEETQYFGPGTRLLVL

SEQ ID NO: 212 - protein, 131 aa, Artificial Sequence
  Description of Artificial Sequence: Synthetic polypeptide
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVRGYNFNKFYFGSGTKLNVKP

SEQ ID NO: 213 - protein, 134 aa, Artificial Sequence
  Description of Artificial Sequence: Synthetic polypeptide
MLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSARGPGEREANTDTQYFGPGTRLTVL

SEQ ID NO: 214 - protein, 136 aa, Artificial Sequence
  Description of Artificial Sequence: Synthetic polypeptide
MATGSRTSLLLAFGLLCLPWLQEASARKEVEQDPGPFNVPEGATVAFNCTYSNSASQSFFWYRQDCRKEPKLLMSVYSSGNEDGRFTAQLNRASQYISLLIRDSKLSDSATYLCVVRGYNFNKFYFGSGTKLNVKP

SEQ ID NO: 215 - protein, 146 aa, Artificial Sequence
  Description of Artificial Sequence: Synthetic polypeptide
MATGSRTSLLLAFGLLCLPCLQEGSAGAVVSQHPSWVICKSGTSVKIECRSLDFQATTMFWYRQFPKQSLMLMATSNEGSKATYEQGVEKDKFLINHASLTLSTLTVTSAHPEDSSFYICSARGPRITDSSTDTQYFGPGTRLTVL

SEQ ID NO: 216 - protein, 136 aa, Artificial Sequence
  Description of Artificial Sequence: Synthetic polypeptide
MATGSRTSLLLAFGLLCLPWLQEASAGQQVMQIPQYQHVQEGEDFTTYCNSSTTLSNIQWYKQRPGGHPVFLIQLVKSGEVKKQKRLTFQFGEAKKNSSLHITATQTTDVGTYFCARFSDGQKLLFARGTMLKVDL

SEQ ID NO: 217 - protein, 141 aa, Artificial Sequence
  Description of Artificial Sequence: Synthetic polypeptide
MATGSRTSLLLAFGLLCLPCLQEGSANAGVTQTPKFRILKIGQSMTLQCTQDMNHNYMYWYRQDPGMGLKLIYYSVGAGITDKGEVPNGYNVSRSTTEDFPLRLELAAPSQTSVYFCASSCRGRGDCTEAFFGQGTRLTVV

SEQ ID NO: 218 - protein, 132 aa, Artificial Sequence
  Description of Artificial Sequence: Synthetic polypeptide
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMSAWASKIIFGSGTRLSIRP

SEQ ID NO: 219 - protein, 134 aa, Artificial Sequence
  Description of Artificial Sequence: Synthetic polypeptide
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSEKGTGRQETQYFGPGTRLLVL

SEQ ID NO: 220 - protein, 136 aa, Artificial Sequence; identical to SEQ ID NO: 216
  Description of Artificial Sequence: Synthetic polypeptide

SEQ ID NO: 221 - protein, 140 aa, Artificial Sequence
  Description of Artificial Sequence: Synthetic polypeptide
MATGSRTSLLLAFGLLCLPCLQEGSANAGVTQTPKFRILKIGQSMTLQCTQDMNHNYMYWYRQDPGMGLKLIYYSVGAGITDKGEVPNGYNVSRSTTEDFPLRLELAAPSQTSVYFCASSNRGGVPDTQYFGPGTRLTVL

SEQ ID NO: 222 - protein, 130 aa, Artificial Sequence
  Description of Artificial Sequence: Synthetic polypeptide
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCGGLGFGNVLHCGSGTQVIVLP

SEQ ID NO: 223 - protein, 130 aa, Artificial Sequence
  Description of Artificial Sequence: Synthetic polypeptide
MLCSLLALLLGTFFGVRSQTIHQWPATLVQPVGSPLSLECTVEGTSNPNLYWYRQAAGRGLQLLFYSVGIGQISSEVPQNLSASRPQDRQFILSSKKLLLSDSGFYLCAWSSLASSYEQYFGPGTRLTVT

SEQ ID NO: 224 - protein, 141 aa, Artificial Sequence
  Description of Artificial Sequence: Synthetic polypeptide
DIQNPEPAVYQLKDPRSQDSTLCLFTDFDSQINVPKTMESGTFITDKTVLDMKAMDSKSNGAIAWSNQTSFTCQDIFKETNACYPSSDVPCDATLTEKSFETDMNLNFQNLSVMGLRILLLKVAGFNLLMTLRLWSSRAKR

SEQ ID NO: 225 - protein, 173 aa, Artificial Sequence
  Description of Artificial Sequence: Synthetic polypeptide
EDLRNVTPPKVSLFEPSKAEIANKQKATLVCLARGFFPDHVELSWWVNGKEVHSGVSTDPQAYKESNYSYCLSSRLRVCATFWHNPRNHFRCQVQFHGLSEEDKWPEGSPKPVTQNISAEAWGRADCGITSASYQQGVLSATILYEILLGKATLYAVLVSTLVVMAMVKRKNS

(19) United States
(12) Patent Application Publication
(10) Pub. No.: US 2023/0227527 A1
(43) Pub. Date: Jul. 20, 2023

(54) TCR-T CELL THERAPY TARGETING EPSTEIN-BARR VIRUS

(71) Applicants: GUANGDONG TCRCURE BIOPHARMA TECHNOLOGY CO., LTD., Guangzhou (CN); TCRCURE BIOPHARMA CORP., Durham, NC (US)

(72) Inventors: Haiyang Wu, Guangzhou (CN); Jie Zhou, Pewaukee, WI (US); Dachuan Deng, Guangzhou (CN); Si Li, Alhambra, CA (US); Lixia Zhao, Cary, NC (US); Lu Zhang, Durham, NC (US); Rui Chen, Durham, NC (US); Paul Bryson, Culver City, CA (US); Poorva Prakash Mudgal, Mebane, NC (US)

(21) Appl. No.: 18/007,662

(22) PCT Filed: Jun. 4, 2021
(86) PCT No.: PCT/CN2021/098487

§ 371 Date: Dec. 1, 2022

(30) Foreign Application Priority Data
Jun. 5, 2020 (WO) PCT/CN2020/094628

(51) Int. Cl. C07K 14/725; C07K 14/74; C07K 16/28; A61K 35/17; A61P 31/22; A61P 35/00; C12N 15/86

(52) U.S. Cl. CPC C07K 14/7051; A61K 35/17; A61P 31/22; A61P 35/00; C07K 14/70539; C07K 16/2818; C12N 15/86; A61K 38/00

(57) ABSTRACT

Provided are T cell receptors that recognize or bind to Epstein-Barr virus (EBV) antigens, genetically engineered cells, and cell-based therapies.

### TECHNICAL FIELD

The present disclosure generally relates to engineered cells and compositions thereof, particularly, T cells comprising genetically engineered T cell receptors (TCRs). Methods for using the compositions to treat cancer are also disclosed herein.

### BACKGROUND

Epstein-Barr virus (EBV) is a member of the herpesvirus family that can infect humans. EBV spreads most commonly through bodily fluids, primarily saliva. EBV can cause infectious mononucleosis, also called mono, and other diseases including cancer. EBV has evolved a life cycle that mimics the natural differentiation pathway of antigen-activated B cells, giving the virus access to its site of latent infection. In proliferating infected B cells, Epstein-Barr virus (EBV) executes a program of gene expression, the “growth” or “latency III” program. This type of latency is found in in vitro EBV-induced lymphoblastoid cell lines (LCLs), in post-transplant lymphoproliferative diseases (Brink AA, 1997, J Clin Pathol 50: 911-918.), as well as in EBV-infected B cells in lymphoid organs during primary and persistent EBV infection, where this program is thought to lead to amplification of EBV load through proliferation of infected cells (Young LS, 2004, Nat Rev Cancer 4: 757-768; Hochberg D, 2004, Proc Natl Acad Sci USA 101: 239-244). Several immunogenic EBV antigens, termed latent membrane proteins (LMP1, LMP2A, LMP2B) and the Epstein-Barr nuclear antigens (EBNA1, -2, -3A, -3B, -3C, -LP), are expressed in latency III EBV-infected B cells.

EBV infection is associated with certain types of cancer. EBV DNA has been found in patients with nasopharyngeal cancer (Mutirangura et al., Clin Cancer Res. 4: 665-9 (1998); Lo et al., Cancer Res. 59: 1188-91 (1999)), lymphomas (Lei et al., Br J Haematol. 111:239-46 (2000); Gallagher et al., Int J Cancer. 84: 442-8 (1999); Dronet et al., J Med Viral. 57: 383-9 (1999)), breast cancer (Bonnet, M. et al., J. Natl. Cancer Inst., 91: 1376-1381 (1999)) and hepatocellular carcinoma (Sugawara, Y. et al., Virology, 256: 196-202 (1999)).

Adoptive cell transfer (ACT), as a modality of immunotherapy against cancer, has demonstrated remarkable success in treating hematologic malignancies and malignant melanoma. One form of ACT, which uses genetically modified T cells expressing a chimeric antigen receptor (CAR) to specifically target a tumor-associated-antigen (TAA), such as CD19 or GD2, has displayed encouraging results in clinical trials for treating such diseases as B cell malignancies. Despite this documented success of CAR-T cell therapy in patients with hematologic malignancies, only modest responses have been observed against solid tumors. Thus, there is a need for efficacious TCR-T cell therapies targeting EBV-induced cancer.

### SUMMARY

The present disclosure provides an engineered T cell, comprising a nucleic acid encoding an anti-LMP2 TCR wherein the anti-LMP2 TCR is a genetically engineered T cell receptor (TCR) that specifically binds to LMP2 in a tumor.

In one aspect, provided herein is a T cell receptor (TCR) or antigen-binding fragment thereof, comprising an alpha chain comprising a variable alpha (Va) region and a beta chain comprising a variable beta (Vb) region. In some embodiments, the Va region comprises a complementarity determining region 1 (CDR1), a complementarity determining region 2 (CDR2), and a complementarity determining region 3 (CDR3). In some embodiments, the CDR3 of the Va region comprises an amino acid sequence X₁GX₂SGYSTL, wherein the X₁ is a E, T, Q, V, or N; the X₂ is a D, G, N, or E. In some embodiments, the Vb region comprises a CDR1, a CDR2, and a CDR3. In some embodiments, the CDR3 of the Vb region comprises an amino acid sequence X₃X₄QGGX₅X₆X₇X₈, wherein the X₃ is a S, T, N, or R; the X₄ is a T, R, Y, G, V, Q, F, S, or P; the X₅ is a N, G, H, T, S, A, I, or W; the X₆ is a Y, N, D, E, R, or I; the X₇ is a G, Q, N, Y; the X₈ is a Y, F, or G.

In some embodiments, the Va region is encoded by a sequence from rearrangement of a human TRAV gene segment and a human TRAJ gene segment, and the Vb region is encoded by a sequence from rearrangement of a human TRBV gene segment, optionally a human TRBD gene segment, and a human TRBJ gene segment. In some embodiments, the TRAV gene segment is TRAV17; the TRAJ gene segment is TRAJ11; the TRBV gene segment is TRBV6-5; the TRBD gene segment is TRBD1 or TRBD2; and the TRBJ gene segment is TRBJ2-1 or TRBJ1-2.

In some embodiments, the Va CDR1 region comprises an amino acid sequence that is at least 80% identical to TSINN (SEQ ID NO: 1), the Va CDR2 region comprises an amino acid sequence that is at least 80% identical to IRSNERE (SEQ ID NO: 2), the Vb CDR1 region comprises an amino acid sequence that is at least 80% identical to MNHEY (SEQ ID NO: 4), and the Vb CDR2 region comprises an amino acid sequence that is at least 80% identical to SVGAGI (SEQ ID NO: 5).

In some embodiments, the Va CDR1 region comprises an amino acid sequence that is at least 80% identical to a selected Va CDR1 amino acid sequence, the Va CDR2 region comprises an amino acid sequence that is at least 80% identical to a selected Va CDR2 amino acid sequence, the Va CDR3 region comprises an amino acid sequence that is at least 80% identical to a selected Va CDR3 amino acid sequence. In some embodiments, the Vb CDR1 region comprises an amino acid sequence that is at least 80% identical to a selected Vb CDR1 amino acid sequence, the Vb CDR2 region comprises an amino acid sequence that is at least 80% identical to a selected Vb CDR2 amino acid sequence, the Vb CDR3 region comprises an amino acid sequence that is at least 80% identical to a selected Vb CDR3 amino acid sequence. In some embodiments, the selected Va CDRs 1, 2, and 3 and Vb CDRs 1, 2, and 3 amino acid sequences are one of the following:
(1) the selected Va CDRs 1, 2, 3 amino acid sequences are set forth in SEQ ID NOs: 1-3, respectively, and the selected Vb CDRs 1, 2, 3 amino acid sequences are set forth in SEQ ID NOs: 4-6, respectively;
(2) the selected Va CDRs 1, 2, 3 amino acid sequences are set forth in SEQ ID NOs: 7-9, respectively, and the selected Vb CDRs 1, 2, 3 amino acid sequences are set forth in SEQ ID NOs: 10-12, respectively;
(3) the selected Va CDRs 1, 2, 3 amino acid sequences are set forth in SEQ ID NOs: 13-15, respectively, and the selected Vb CDRs 1, 2, 3 amino acid sequences are set forth in SEQ ID NOs: 16-18, respectively;
(4) the selected Va CDRs 1, 2, 3 amino acid sequences are set forth in SEQ ID NOs: 19-21, respectively, and the selected Vb CDRs 1, 2, 3 amino acid sequences are set forth in SEQ ID NOs: 22-24, respectively;
(5) the selected Va CDRs 1, 2, 3 amino acid sequences are set forth in SEQ ID NOs: 25-27, respectively, and the selected Vb CDRs 1, 2, 3 amino acid sequences are set forth in SEQ ID NOs: 28-30, respectively;
(6) the selected Va CDRs 1, 2, 3 amino acid sequences are set forth in SEQ ID NOs: 31-33, respectively, and the selected Vb CDRs 1, 2, 3 amino acid sequences are set forth in SEQ ID NOs: 34-36, respectively;
(7) the selected Va CDRs 1, 2, 3 amino acid sequences are set forth in SEQ ID NOs: 37-39, respectively, and the selected Vb CDRs 1, 2, 3 amino acid sequences are set forth in SEQ ID NOs: 40-42, respectively;
(8) the selected Va CDRs 1, 2, 3 amino acid sequences are set forth in SEQ ID NOs: 43-45, respectively, and the selected Vb CDRs 1, 2, 3 amino acid sequences are set forth in SEQ ID NOs: 46-48, respectively;
(9) the selected Va CDRs 1, 2, 3 amino acid sequences are set forth in SEQ ID NOs: 49-51, respectively, and the selected Vb CDRs 1, 2, 3 amino acid sequences are set forth in SEQ ID NOs: 52-54, respectively;
(10) the selected Va CDRs 1, 2, 3 amino acid sequences are set forth in SEQ ID NOs: 61-63, respectively, and the selected Vb CDRs 1, 2, 3 amino acid sequences are set forth in SEQ ID NOs: 64-66, respectively;
(11) the selected Va CDRs 1, 2, 3 amino acid sequences are set forth in SEQ ID NOs: 67-69, respectively, and the selected Vb CDRs 1, 2, 3 amino acid sequences are set forth in SEQ ID NOs: 70-72, respectively;
(12) the selected Va CDRs 1, 2, 3 amino acid sequences are set forth in SEQ ID NOs: 73-75, respectively, and the selected Vb CDRs 1, 2, 3 amino acid sequences are set forth in SEQ ID NOs: 76-78, respectively;
(13) the selected Va CDRs 1, 2, 3 amino acid sequences are set forth in SEQ ID NOs: 79-81, respectively, and the selected Vb CDRs 1, 2, 3 amino acid sequences are set forth in SEQ ID NOs: 82-84, respectively; or
(14) the selected Va CDRs 1, 2, 3 amino acid sequences are set forth in SEQ ID NOs: 85-87, respectively, and the selected Vb CDRs 1, 2, 3 amino acid sequences are set forth in SEQ ID NOs: 88-90, respectively.

In one aspect, provided herein is a T cell receptor (TCR) or antigen-binding fragment thereof, comprising an alpha chain comprising a variable alpha (Va) region. In some embodiments, the Va region comprises a complementarity determining region 1 (CDR-1), a complementarity determining region 2 (CDR-2), and a complementarity determining region 3 (CDR-3). In some embodiments, the Va CDR1 region comprises an amino acid sequence that is at least 80% identical to SEQ ID NO: 55, the Va CDR2 region comprises an amino acid sequence that is at least 80% identical to SEQ ID NO: 56, the Va CDR3 region comprises an amino acid sequence that is at least 80% identical to SEQ ID NO: 57. In some embodiments, provided herein is the T cell receptor (TCR) or antigen-binding fragment thereof, further comprising a beta chain comprising a variable beta (Vb) region. In some embodiments, the Vb region comprises a complementarity determining region 1 (CDR-1), a complementarity determining region 2 (CDR-2), and a complementarity determining region 3 (CDR-3). In some embodiments, the Vb CDR1 region comprises an amino acid sequence that is at least 80% identical to SEQ ID NO: 58, the Vb CDR2 region comprises an amino acid sequence that is at least 80% identical to SEQ ID NO: 59, the Vb CDR3 region comprises an amino acid sequence that is at least 80% identical to SEQ ID NO: 60.

In some embodiments, the TCR or antigen-binding fragment thereof binds to or recognizes a peptide epitope of LMP2 (FLYALALLL) (SEQ ID NO: 139) that is presented by a major histocompatibility complex (MHC) molecule.

In some embodiments, the MHC molecule is an HLA-A2 molecule.

In some embodiments, the Va region is encoded by a sequence from rearrangement of a human TRAV gene segment and a human TRAJ gene segment, and the Vb region is encoded by a sequence from rearrangement of a human TRBV gene segment, optionally a human TRBD gene segment, and a human TRBJ gene segment. In some embodiments, the TRAV gene segment is TRAV21; the TRAJ gene segment is TRAJ33; the TRBV gene segment is TRBV10-2; the TRBD gene segment is TRBD1 or TRBD2; and the TRBJ gene segment is TRBJ2-7.

In some embodiments, the Va CDR1 region comprises an amino acid sequence that is at least 80% identical to SEQ ID NO: 143, the Va CDR2 region comprises an amino acid sequence that is at least 80% identical to SEQ ID NO: 144, the Va CDR3 region comprises an amino acid sequence that is at least 80% identical to SEQ ID NO: 145; and in some embodiments, the Vb CDR1 region comprises an amino acid sequence that is at least 80% identical to SEQ ID NO: 146, the Vb CDR2 region comprises an amino acid sequence that is at least 80% identical to SEQ ID NO: 147, the Vb CDR3 region comprises an amino acid sequence that is at least 80% identical to SEQ ID NO: 148.

In some embodiments, the TCR or antigen-binding fragment thereof binds to or recognizes a peptide epitope of LMP2 CLGGLLTMV (SEQ ID NO: 167) and/or SLGGLLTMV (SEQ ID NO: 168) that is presented by a major histocompatibility complex (MHC) molecule.

In some embodiments, the MHC molecule is an HLA-A2 molecule.

In one aspect, provided herein is a T cell receptor (TCR) or antigen-binding fragment thereof, comprising an alpha chain comprising a variable alpha (Va) region. In some embodiments, the Va region comprises a complementarity determining region 1 (CDR-1), a complementarity determining region 2 (CDR-2), and a complementarity determining region 3 (CDR-3). In some embodiments, the Va CDR1 region comprises an amino acid sequence that is at least 80% identical to a selected Va CDR1 amino acid sequence, the Va CDR2 region comprises an amino acid sequence that is at least 80% identical to a selected Va CDR2 amino acid sequence, the Va CDR3 region comprises an amino acid sequence that is at least 80% identical to a selected Va CDR3 amino acid sequence. In some embodiments, provided herein is the TCR or antigen-binding fragment thereof, further comprising a beta chain comprising a variable beta (Vb) region. In some embodiments, the Vb region comprises a complementarity determining region 1 (CDR-1), a complementarity determining region 2 (CDR-2), and a complementarity determining region 3 (CDR-3). In some embodiments, the Vb CDR1 region comprises an amino acid sequence that is at least 80% identical to a selected Vb CDR1 amino acid sequence, the Vb CDR2 region comprises an amino acid sequence that is at least 80% identical to a selected Vb CDR2 amino acid sequence, the Vb CDR3 region comprises an amino acid sequence that is at least 80% identical to a selected Vb CDR3 amino acid sequence. In some embodiments, the selected Va CDRs 1, 2, and 3 and Vb CDRs 1, 2, and 3 amino acid sequences are one of the following:
(1) the selected Va CDRs 1, 2, 3 amino acid sequences are set forth in SEQ ID NOs: 91, 92, 93, respectively, and the selected Vb CDRs 1, 2, 3 amino acid sequences are set forth in SEQ ID NOs: 94, 95, 96, respectively;
(2) the selected Va CDRs 1, 2, 3 amino acid sequences are set forth in SEQ ID NOs: 97, 98, 99, respectively, and the selected Vb CDRs 1, 2, 3 amino acid sequences are set forth in SEQ ID NOs: 100, 101, 102, respectively;
(3) the selected Va CDRs 1, 2, 3 amino acid sequences are set forth in SEQ ID NOs: 103, 104, 105, respectively, and the selected Vb CDRs 1, 2, 3 amino acid sequences are set forth in SEQ ID NOs: 106, 107, 108, respectively; and
(4) the selected Va CDRs 1, 2, 3 amino acid sequences are set forth in SEQ ID NOs: 149, 150, and 151, respectively, and the selected Vb CDRs 1, 2, 3 amino acid sequences are set forth in SEQ ID NOs: 152, 153, and 154, respectively; and
(5) the selected Va CDRs 1, 2, 3 amino acid sequences are set forth in SEQ ID NOs: 155, 156, and 157, respectively, and the selected Vb CDRs 1, 2, 3 amino acid sequences are set forth in SEQ ID NOs: 158, 159, and 160, respectively.

In some embodiments, the Va region is encoded by a sequence from rearrangement of a human TRAV gene segment and a human TRAJ gene segment, and the Vb region is encoded by a sequence from rearrangement of a human TRBV gene segment, optionally a human TRBD gene segment, and a human TRBJ gene segment. In some embodiments, the TRAV gene segment is TRAV4, TRAV25, TRAV22, or TRAV6; the TRAJ gene segment is TRAJ23, TRAJ47, TRAJ29, TRAJ43, or TRAJ11; the TRBV gene segment is TRBV12-4, TRBV12-3, TRBV11-2, TRBV4-1, or TRBV11-2; the TRBD gene segment is TRBD2; and the TRBJ gene segment is TRBJ1-1, TRBJ2-1, TRBJ2-5, or TRBJ2-7.

In some embodiments, the TCR or antigen-binding fragment thereof binds to or recognizes a peptide epitope of LMP2 (SSCSSCPLSK) (SEQ ID NO: 140) that is presented by a major histocompatibility complex (MHC) molecule.

In some embodiments, the MHC molecule is an HLA-A11 molecule.

In one aspect, provided herein is a T cell receptor (TCR) or antigen-binding fragment thereof, comprising an alpha chain comprising a variable alpha (Va) region. In some embodiments, the Va region comprises a complementarity determining region 1 (CDR-1), a complementarity determining region 2 (CDR-2), and a complementarity determining region 3 (CDR-3). In some embodiments, the Va CDR1 region comprises an amino acid sequence that is at least 80% identical to a selected Va CDR1 amino acid sequence, the Va CDR2 region comprises an amino acid sequence that is at least 80% identical to a selected Va CDR2 amino acid sequence, the Va CDR3 region comprises an amino acid sequence that is at least 80% identical to a selected Va CDR3 amino acid sequence. In some embodiments, provided herein is the TCR or antigen-binding fragment thereof, further comprising a beta chain comprising a variable beta (Vb) region. In some embodiments, the Vb region comprises a complementarity determining region 1 (CDR-1), a complementarity determining region 2 (CDR-2), and a complementarity determining region 3 (CDR-3). In some embodiments, the Vb CDR1 region comprises an amino acid sequence that is at least 80% identical to a selected Vb CDR1 amino acid sequence, the Vb CDR2 region comprises an amino acid sequence that is at least 80% identical to a selected Vb CDR2 amino acid sequence, the Vb CDR3 region comprises an amino acid sequence that is at least 80% identical to a selected Vb CDR3 amino acid sequence. In some embodiments, the selected Va CDRs 1, 2, and 3 and Vb CDRs 1, 2, and 3 amino acid sequences are one of the following:
(1) the selected Va CDRs 1, 2, 3 amino acid sequences are set forth in SEQ ID NOs: 109, 110, 111, respectively, and the selected Vb CDRs 1, 2, 3 amino acid sequences are set forth in SEQ ID NOs: 112, 113, 114, respectively; or
(2) the selected Va CDRs 1, 2, 3 amino acid sequences are set forth in SEQ ID NOs: 115, 116, 117, respectively, and the selected Vb CDRs 1, 2, 3 amino acid sequences are set forth in SEQ ID NOs: 118, 119, 120, respectively.

In some embodiments, the Va region is encoded by a sequence from rearrangement of a human TRAV gene segment and a human TRAJ gene segment, and the Vb region is encoded by a sequence from rearrangement of a human TRBV gene segment, optionally a human TRBD gene segment, and a human TRBJ gene segment. In some embodiments, the TRAV gene segment is TRAV12-1; the TRAJ gene segment is TRAJ21; the TRBV gene segment is TRBV20-1; the TRBD gene segment is TRBD1; and the TRBJ gene segment is TRBJ2-5 or TRBJ2-3.

In some embodiments, the TCR or antigen-binding fragment thereof binds to or recognizes a peptide epitope of LMP2 (IYVLVMLVL) (SEQ ID NO: 141) that is presented by a major histocompatibility complex (MHC) molecule.

In one aspect, provided herein is a T cell receptor (TCR) or antigen-binding fragment thereof, comprising an alpha chain comprising a variable alpha (Va) region. In some embodiments, the Va region comprises a complementarity determining region 1 (CDR-1), a complementarity determining region 2 (CDR-2), and a complementarity determining region 3 (CDR-3). In some embodiments, the Va CDR1 region comprises an amino acid sequence that is at least 80% identical to a selected Va CDR1 amino acid sequence, the Va CDR2 region comprises an amino acid sequence that is at least 80% identical to a selected Va CDR2 amino acid sequence, the Va CDR3 region comprises an amino acid sequence that is at least 80% identical to a selected Va CDR3 amino acid sequence. In some embodiments, provided herein is the TCR or antigen-binding fragment thereof, further comprising a beta chain comprising a variable beta (Vb) region. In some embodiments, the Vb region comprises a complementarity determining region 1 (CDR-1), a complementarity determining region 2 (CDR-2), and a complementarity determining region 3 (CDR-3). In some embodiments, the Vb CDR1 region comprises an amino acid sequence that is at least 80% identical to a selected Vb CDR1 amino acid sequence, the Vb CDR2 region comprises an amino acid sequence that is at least 80% identical to a selected Vb CDR2 amino acid sequence, the Vb CDR3 region comprises an amino acid sequence that is at least 80% identical to a selected Vb CDR3 amino acid sequence. In some embodiments, the selected Va CDRs 1, 2, and 3 and Vb CDRs 1, 2, and 3 amino acid sequences are one of the following:
(1) the selected Va CDRs 1, 2, 3 amino acid sequences are set forth in SEQ ID NOs: 121, 122, 123, respectively, and the selected Vb CDRs 1, 2, 3 amino acid sequences are set forth in SEQ ID NOs: 124, 125, 126, respectively;
(2) the selected Va CDRs 1, 2, 3 amino acid sequences are set forth in SEQ ID NOs: 127, 128, 129, respectively, and the selected Vb CDRs 1, 2, 3 amino acid sequences are set forth in SEQ ID NOs: 130, 131, 132, respectively;
(3) the selected Va CDRs 1, 2, 3 amino acid sequences are set forth in SEQ ID NOs: 133, 134, 135, respectively, and the selected Vb CDRs 1, 2, 3 amino acid sequences are set forth in SEQ ID NOs: 136, 137, 138, respectively; or
(4) the selected Va CDRs 1, 2, 3 amino acid sequences are set forth in SEQ ID NOs: 161, 162, and 163, respectively, and the selected Vb CDRs 1, 2, 3 amino acid sequences are set forth in SEQ ID NOs: 164, 165, and 166, respectively.

In some embodiments, the Va region is encoded by a sequence from rearrangement of a TCR alpha variable (TRAV) gene segment and a TCR alpha joining (TRAJ) gene segment, and the Vb region is encoded by a sequence from rearrangement of a TCR beta variable (TRBV) gene segment, optionally a TCR beta diversity (TRBD) gene segment, and a TCR beta joining (TRBJ) gene segment. In some embodiments, the TRAV gene segment is TRAV25, TRAV12-3, or TRAV21; the TRAJ gene segment is TRAJ16, TRAJ3, TRAJ35; the TRBV gene segment is TRBV6-6, TRBV24-1, or TRBV30; the TRBD gene segment is TRBD1 or TRBD2; and the TRBJ gene segment is TRBJ1-1, TRBJ2-5, TRBJ2-3, or TRBJ2-7.

In some embodiments, the TCR or antigen-binding fragment thereof binds to or recognizes a peptide epitope of LMP2 (TYGPVFMCL) (SEQ ID NO: 142) that is presented by a major histocompatibility complex (MHC) molecule.

In some embodiments, the MHC molecule is an HLA-A24 molecule.

In some embodiments, the alpha chain comprises a mouse alpha chain constant region, and the beta chain comprises a mouse beta chain constant region.

In some embodiments, the alpha chain comprises a human alpha chain constant region, and the beta chain comprises a human beta chain constant region.

In some embodiments, the TCR or antigen-binding fragment thereof, when expressed on the surface of a T cell, stimulates cytotoxic activity against a target cancer cell. In some embodiments, the target cancer cell comprises a nucleic acid sequence encoding LMP2 or expresses LMP2.

In one aspect, provided herein is a vector comprising a nucleic acid encoding TCR or antigen-binding fragment thereof as described herein.

In one aspect, provided herein is a vector comprising: a) a first nucleic acid sequence encoding a TCR alpha chain comprising an alpha chain variable region of a human anti-LMP2 TCR and an alpha chain constant region; and b) a second nucleic acid sequence encoding a TCR beta chain comprising a beta chain variable region of the human anti-LMP2 TCR and a beta chain constant region. In some embodiments, the TCR alpha chain and the TCR beta chain form the TCR or antigen-binding fragment thereof as described herein.

In some embodiments, the first nucleic acid sequence and the second nucleic acid sequence is linked by a linker sequence. In some embodiments, the linker sequence is a P2A sequence.

In some embodiments, the vector is an expression vector, a viral vector, a retroviral vector, or a lentiviral vector. In some embodiments, the retroviral vector is pMP71.

In one aspect, provided herein is an engineered cell comprising the vector as described herein.

In one aspect, provided herein is an engineered cell, comprising the TCR or antigen-binding fragment thereof as described herein.

In some embodiments, the TCR or antigen binding fragment thereof is heterologous to the cell.

In some embodiments, the engineered cell is a cell line.

In some embodiments, the engineered cell is a primary cell obtained from a subject (e.g., a human subject).

In some embodiments, the engineered cell is a T cell. In some embodiments, the T-cell is isolated from a human subject. In some embodiments, the T cell is CD8+. In some embodiments, the T cell is CD4+.

In some embodiments, the engineered cell expresses a bifunctional trap protein. In some embodiments, the bifunctional trap protein targets a checkpoint inhibitor (e.g., PD-1) and a member of the transforming growth factor beta family (e.g., TGF-β).

In some embodiments, the engineered cell expresses an antibody or antigen-binding fragment thereof targeting a checkpoint inhibitor (e.g., PD-1).

In some embodiments, the engineered cell expresses a protein that binds to a member of the transforming growth factor beta family (e.g., TGF-β).

In one aspect, provided herein is a method for producing the engineered cell, comprising introducing the vector as described herein into the cell in vitro or ex vivo. In some embodiments, the introducing step is carried out by transduction.

In one aspect, provided herein is a method of treating a disease or a disorder, comprising administering the engineered cell as described herein to a subject having a disease or disorder associated with EBV. In some embodiments, the disease or disorder associated with EBV is a cancer.

In one aspect, provided herein is a method of treating a tumor in a subject, the method comprising administering to the subject in need thereof an engineered T cell, comprising a nucleic acid encoding the TCR or antigen-binding fragment thereof as described herein that specifically binds to an antigen in the tumor.

In one aspect, provided herein is a method of treating a tumor in a subject, the method comprising administering to the subject in need thereof (a) an engineered T cell, comprising: a nucleic acid encoding the TCR or antigen-binding fragment thereof as described herein that specifically binds to an antigen in a tumor; and (b) either one or both of a checkpoint inhibitor and a protein that binds to a member of the transforming growth factor beta family (e.g., TGF-β).

In one aspect, provided herein is a method of treating a tumor in a subject, the method comprising administering to the subject in need thereof an engineered T cell, comprising: a nucleic acid encoding (a) the TCR or antigen-binding fragment thereof as described herein that specifically binds to an antigen in a tumor; and (b) a bifunctional trap protein that targets a checkpoint inhibitor and a member of the transforming growth factor beta family (e.g., TGF-β).

In some embodiments, the tumor is an EBV-associated tumor.

In some embodiments, the cancer or the EBV-associated tumor as described herein is Burkitt’s lymphoma, immunosuppressive lymphoma, diffuse large B-cell lymphoma, diffuse large B-cell lymphoma associated with chronic inflammation, lymphomatoid granulomatosis, plasmablastic lymphoma, primary effusion lymphoma, post-transplant lymphoproliferative disorder, nasopharyngeal carcinoma, gastric adenocarcinoma, lymphoepithelioma-associated carcinoma, immunodeficiency-related leiomyosarcoma, or Hodgkin’s lymphoma.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

Exemplary embodiments are illustrated in referenced figures. It is intended that the embodiments and figures disclosed herein are to be considered illustrative rather than restrictive.

FIG. 1 is a schematic diagram showing a pMP71 retroviral vector construct. P2A encodes a 2A self-cleaving peptide; Va encodes the variable region of the alpha chain of a human anti-LMP2 TCR; Vb encodes the variable region of the beta chain of the same human anti-LMP2 TCR; Ca encodes the constant region of a mouse TCR alpha chain; Cb encodes the constant region of the mouse TCR beta chain. Ψ indicates packaging sequences for the virus. 5′LTR and 3′LTR are long terminal repeats.

FIG. 2 is a table showing sequences of HLA-A2 typed anti-LMP2 TCRs. CDR1α, CDR2α, and CDR3α are CDR1, CDR2 and CDR3 of the TCR alpha chain, respectively. CDR1β, CDR2β, and CDR3β are CDR1, CDR2 and CDR3 of the TCR beta chain, respectively. TRA_VJ are the rearranged V and J segments encoding the alpha chain of the anti-LMP2 TCR. TRB_VDJ are the rearranged V, D, and J segments encoding the beta chain of the anti-LMP2 TCR. Target epitope shows the amino acid position of the TCR-targeting epitope from the LMP2 protein.

FIG. 3 is a table showing sequences of HLA-A11 typed anti-LMP2 TCRs.

FIG. 4 is a table showing sequences of HLA-A24 typed anti-LMP2 TCRs.

FIGS. 5A-5C are a set of graphs showing the in vitro expression of HLA-A2 typed anti-LMP2 TCRs in Jurkat cells transduced with the construct encoding the recombinant TCR. CD3 was stained and a viable CD3⁺ lymphocyte gating strategy was used. Untransduced (UT) cells were used as a negative control.

FIG. 5D is a set of graphs showing the in vitro expression of HLA-A2 typed anti-LMP2 TCRs in primary human T cells transduced with the construct encoding the recombinant TCR. CD3 and CD8 were stained simultaneously and a viable CD3⁺ lymphocyte gating strategy was used. Untransduced (UT) cells were used as a negative control.

FIG. 5E is a graph showing the in vitro expression of HLA-A2 typed anti-LMP2 TCR L208 in primary human T cells transduced with the construct encoding the recombinant TCR. CD3 and CD8 were stained simultaneously and a viable CD3⁺ lymphocyte gating strategy was used. Untransduced (UT) cells were used as a negative control.

FIGS. 6A-6C are graphs showing the surface CD69 expression of antigen-specific stimulated TCR-Jurkat cells. CD3⁺ TCR⁺ cells were analyzed. Untransduced (UT) cells were used as a negative control (Ctrl).

FIG. 6D is a set of graphs showing the intracellular IFN-y expression in antigen-specific stimulated TCR-T cells. CD3⁺ CD8⁺ cells were analyzed. Untransduced (UT) cells were used as a negative control.

FIG. 6E is a graph showing the intracellular IFN-y expression in antigen-specific stimulated TCR-T cells. CD3+ CD8+ cells were analyzed. Untransduced (UT) cells were used as a negative control. The antigen is a mutant peptide (SEQ ID NO: 168) from the LMP2 protein.

FIGS. 6F-6G show activation curves of TCR-T cells expressing the HLA-A2 typed anti-LMP2 TCR L208. EC50 was determined for TCR-T cells expressing the L208 recombinant TCR using increasing concentrations of the cognate wild-type or mutant LMP2 peptide.

FIGS. 7A-7B are graphs showing the in vitro expression of HLA-A11 typed anti-LMP2 TCRs in primary T cells transduced with the construct encoding the recombinant TCR. CD3 and CD8 were stained simultaneously and a viable CD3+ lymphocyte gating strategy was used. Untransduced (UT) cells were used as a negative control.

FIGS. 8A-8B are graphs showing the intracellular IFN-y expression in antigen-specific stimulated TCR-T cells. CD3⁺ CD8⁺ cells were analyzed. Untransduced (UT) cells were used as a negative control.

FIGS. 8C-8D are graphs showing the surface CD69 expression in antigen-specific stimulated TCR-T cells. CD3+ CD8+ cells were analyzed. Untransduced (UT) cells were used as a negative control.

FIG. 9 shows activation curves of TCR-T cells expressing the HLA-A11 typed anti-LMP2 TCRs. EC50 was determined for TCR-T cells expressing the L11-3 or L11-6 recombinant TCR using increasing concentrations of the cognate LMP2 peptide.

FIG. 10A is a set of graphs showing the in vitro expression of HLA-A24 typed anti-LMP2 TCRs in primary human T cells transduced with the construct encoding the recombinant TCR. CD3 and CD8 were stained simultaneously and a viable CD3⁺ lymphocyte gating strategy was used. Untransduced (UT) cells were used as a negative control.

FIG. 10B is a set of graphs showing the in vitro expression of HLA-A24 typed anti-LMP2 TCRs in Jurkat cells transduced with the construct encoding the recombinant TCR. CD3 was stained and a viable CD3⁺ lymphocyte gating strategy was used. A negative control (Ctrl) was used.

FIG. 10C is a set of graphs showing the in vitro expression of HLA-A24 typed anti-LMP2 TCRs in primary human T cells transduced with the construct encoding the recombinant TCR. CD3 and CD8 were stained simultaneously and a viable CD3⁺ lymphocyte gating strategy was used. Untransduced (UT) cells were used as a negative control.

FIGS. 11A-11C are a set of graphs showing the intracellular IFN-y expression in antigen-specific stimulated TCR-T cells. CD3⁺ CD8⁺ cells were analyzed. Untransduced (UT) cells were used as a negative control.

FIG. 11D is a set of graphs showing the surface CD69 expression of antigen-specific stimulated TCR-Jurkat cells. CD3⁺ TCR⁺ cells were analyzed. A negative control (Ctrl) was used..

FIG. 12A shows activation curves of TCR-T cells expressing the HLA-A24 typed anti-LMP2 TCRs. EC50 was determined for TCR-T cells expressing the L24-49 or L24-2 recombinant TCR using increasing concentrations of the cognate LMP2 peptide.

FIG. 12B shows activation curves of TCR-T cells expressing the HLA-A24 typed anti-LMP2 TCRs. EC50 was determined for TCR-T cells expressing the L24-3 or L24-13 recombinant TCR using increasing concentrations of the cognate LMP2 peptide.

FIG. 13 shows the motif analysis using TCRdist method. A clone cluster was identified (indicated with a rectangle), which comprises 30 TCR clones. Sequence alignment was performed using 14 test clones from the cluster. Motifs within CDR3 of TRA and CDR3 of TRB are highlighted.

FIG. 14 shows the motif analysis based on machine learning method (Support Vector Machine). The top left table shows an example of the produced motifs and their counts with motif length of 3 or 4. In the top right. On the right top table, an example of input file was shown. For each positive or negative TCR clone, a Boolean value of 1 was assigned if it contained that motif, or 0 if it does not contain that motif. In the bottom table, Area under ROC Curve (AUC) values and Support Vector Machine (SVM) importance scores were calculated for the top ranked motifs.

FIG. 15 shows the Fisher exact and Cramer’s V test results. P values, adjusted P values by the Fisher exact test, and Cramer importance scores by the Cramer’s V test were calculated for the top ranked motifs.

FIG. 16A shows highly enriched motifs within CDR3 of the TCR beta chain.

FIG. 16B shows highly enriched motifs within CDR3 of the TCR alpha chain.

FIG. 17A shows Receiver Operating Characteristics (ROC) curve of TCR beta chain motif “QGG”. The sensitivity indicates the proportion of actual positives that are correctly identified by having the motif QGG. Specificity indicates the proportion of actual negatives that are correctly identified by not having the motif QGG.

FIG. 17B shows ROC curve of TCR alpha chain motif “YST”.

FIG. 17C shows ROC curve of TCR alpha chain motif “SGY”.

FIG. 18 provides sequences as described in the disclosure.

### DETAILED DESCRIPTION

The following embodiments and aspects thereof are described and illustrated in conjunction with systems, compositions, and methods which are meant to be exemplary and illustrative, not limiting in scope.

### Definitions

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

As used herein, the term “about” refers to a measurable value such as an amount, a time duration, and the like, and encompasses variations of ±20%, ±10%, ±5%, ±1%, ±0.5% or ±0.1% from the specified value.

As used herein, the term “antibody” refers to any antigen-binding molecule that contains at least one (e.g., one, two, three, four, five, or six) immunoglobulin complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′)2, and Fv fragments.

As used herein, “single chain variable fragment”, “single-chain antibody variable fragments” or “scFv” antibodies refer to forms of antibodies comprising the variable regions of only the heavy (VH) and light (VL) chains, connected by a linker peptide. The scFvs are capable of being expressed as a single chain polypeptide. The scFvs retain the specificity of the intact antibody from which it is derived. The light and heavy chain variable regions can be in any order, for example, VH-linker-VL or VL-linker-VH, so long as the specificity of the scFv to the target antigen is retained.

The term “binding protein” refers to protein binding domains (such as cytokine, cytokine receptors), antibody fragments (such as Fab, scFv, diabody, variable domain derived binders, VHH nanobody), alternative scaffold derived protein binding domains (such as Fn3 variants, ankyrin repeat variants, centyrin variants, avimers, affibody) or any protein recognizing specific antigens.

As used herein, the term “antigen” refers to a molecule capable of being bound by an antibody or a T cell receptor (TCR) if presented by MHC molecules. The term “antigen”, as used herein, also encompasses T-cell epitopes which are recognized by T-cell receptors. This recognition can cause activation of T-cells and subsequent effector mechanisms such as proliferation of the T cells, cytokine secretion, etc.

As used herein, the term “EBV antigen” refers to a polypeptide molecule derived from Epstein-Barr virus (EBV). EBV antigen includes, but is not limited to, the latent membrane proteins (LMP1, LMP2A, and LMP2B) and the Epstein-Barr nuclear antigens (EBNA1, -2, -3A, -3B, -3C, -LP).

As used herein, the term “peripheral blood cell subtypes” refers to cell types normally found in the peripheral blood including, but not limited to, eosinophils, neutrophils, T cells, monocytes, K cells, granulocytes, and B cells.

As used herein, the term “T cell” includes CD4+ T cells and CD8+ T cells. The term T cell also includes both T helper 1 type T cells and T helper 2 type T cells. T cells express a cell surface receptor that recognizes a specific antigenic moiety on the surface of a target cell. The cell surface receptor may be a wild type or recombinant T cell receptor (TCR), a chimeric antigen receptor (CAR), or any other surface receptor capable of recognizing an antigenic moiety that is associated with the target cell. Typically, a TCR has two protein chains (alpha-and beta- chain), which bind to specific peptides presented by an MHC protein on the surface of certain cells. TCRs recognize peptides in the context of MHC molecules expressed on the surface of a target cell. In some embodiments, TCRs can also recognize cancer antigens presented directly on the surface of cancer cells.

“Genetically modified cells”, “engineered cells”, “genetically engineered cells” or “modified cells” as used herein refer to cells that are genetically modified. In some embodiments, the genetically engineered cells can express genetically engineered antigen receptors and/or checkpoint inhibitors. In some embodiments, the genetically modified cells comprise vectors that encode a genetically engineered TCR and/or vectors that encode one or more checkpoint inhibitors. In some embodiments, the genetically modified cells comprise a vector that encodes a genetically engineered TCR and one or more checkpoint inhibitors. In one embodiment, the genetically modified cell is a T lymphocyte (T cell). In some embodiments, the genetically modified cell is a Natural Killer (NK) cell.

As used herein, the term “vector”, “cloning vector” or “expression vector” refers to a vehicle by which a polynucleotide sequence (e.g. a foreign gene) can be introduced into a host cell. Vectors include plasmids, phages, viruses, etc. Most popular type of vector is a “plasmid”, which refers to a closed circular double stranded DNA loop into which additional DNA segments comprising gene of interest may be ligated. Another type of vector is a viral vector, in which a nucleic acid construct to be transported is ligated into the viral genome. Viral vectors are capable of autonomous replication in a host cell into which they are introduced or may integrate themselves into the genome of a host cell and thereby are replicated along with the host genome. Moreover, certain vectors are capable of directing the expression of genes to which they are operatively linked. Such vectors are referred to herein as “recombinant expression vectors” or simply “expression vectors”. Some other forms of expression vectors can be as used, such as viral vectors (e.g., replication defective retroviruses, adenoviruses and adeno-associated viruses),

As used herein, the term “retroviral vector” or “recombinant retroviral vector” refers to a nucleic acid construct which carries, and within certain embodiments, is capable of directing the expression of a nucleic acid molecule of interest. A retrovirus is present in the RNA form in its viral capsule and forms a double-stranded DNA intermediate when it replicates in the host cell. Similarly, retroviral vectors are present in both RNA and double-stranded DNA forms, both of which forms are included in the term “retroviral vector” and “recombinant retroviral vector”. The term “retroviral vector” and “recombinant retroviral vector” also encompass the DNA form which contains a recombinant DNA fragment and the RNA form containing a recombinant RNA fragment. The vectors can include at least one transcriptional promoter/enhancer, or other elements which control gene expression. Such vectors can also include a packaging signal, long terminal repeats (LTRs) or portion thereof, and positive and negative strand primer binding sites appropriate to the retrovirus used (if these are not already present in the retroviral vector). Optionally, the vectors can also include a signal which directs polyadenylation, selectable markers such as Ampicillin resistance, Neomycin resistance, TK, hygromycin resistance, phleomycin resistance histidinol resistance, or DHFR, as well as one or more restriction sites and a translation termination sequence. By way of example, such vectors can include a 5′ LTR, a leading sequence, a tRNA binding site, a packaging signal, an origin of second strand DNA synthesis, and a 3′ LTR or a portion thereof.

“Linker” (L) or “linker domain” or “linker region” as used herein refers to an oligo- or polypeptide region from about 1 to 100 amino acids in length, which links together any of the domains/regions of the TCR as described herein. Linkers can be composed of flexible residues like glycine and serine so that the adjacent protein domains are free to move relative to one another. Longer linkers can be used when it is desirable to ensure that two adjacent domains do not sterically interfere with one another. Linkers may be cleavable or non-cleavable. Examples of cleavable linkers include 2A linkers (for example T2A), 2A-like linkers or functional equivalents thereof and combinations thereof. In some embodiments, the linkers include the picornaviral 2A-like linker, CHYSEL sequences of porcine teschovirus (P2A), Thosea asigna virus (T2A) or combinations, variants and functional equivalents thereof. In other embodiments, the linker sequences may comprise Asp-Val/Ile-Glu-X-Asn-Pro-Gly(2A)-Pro(2B) motif, which results in cleavage between the 2A glycine and the 2B proline.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, a “subject” is a mammal, such as a human or other animal, and typically is human. In some embodiments, the subject, e.g., patient, to whom the cells, cell populations, or compositions are administered is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent or a mouse.

The term “control” refers to any reference standard suitable to provide a comparison to the test sample.

As used herein, the term “inhibit” refers to any decrease in, for example a particular action, function, or interaction. For example, a biological function, such as the function of a protein and/or binding of one protein to another, is inhibited if it is decreased as compared to a reference state, such as a control like a wild-type state or a state in the absence of an applied agent. For example, the binding of a PD-1 protein to one or more of its ligands, such as PD-L1 and/or PD-L2, and/or resulting PD-1 signaling and immune effects is inhibited or deficient if the binding, signaling, and other immune effects are decreased due to contact with an agent, such as an anti-PD-1 antibody, in comparison to when the PD-1 protein is not contacted with the agent. Such inhibition or deficiency can be induced, such as by application of agent at a particular time and/or place, or can be constitutive, such as by continual administration. Such inhibition or deficiency can also be partial or complete (e.g., essentially no measurable activity in comparison to a reference state, such as a control like a wild-type state). Essentially complete inhibition or deficiency is referred to as blocked.

“Conditions” and “disease conditions,” as used herein may include, cancers, tumors or infectious diseases. In exemplary embodiments, the conditions include but are in no way limited to any form of malignant neoplastic cell proliferative disorders or diseases. In exemplary embodiments, conditions include any one or more of kidney cancer, melanoma, prostate cancer, breast cancer, glioblastoma, lung cancer, colon cancer, or bladder cancer.

“Cancer” and “cancerous” refers to or describe the physiological condition in mammals that is typically characterized by unregulated cell growth. The term “cancer” is meant to include all types of cancerous growths or oncogenic processes, metastatic tissues or malignantly transformed cells, tissues, or organs, irrespective of histopathologic type or stage of invasiveness. Examples of solid tumors include malignancies, e.g., sarcomas, adenocarcinomas, and carcinomas, of the various organ systems, such as those affecting liver, lung, breast, lymphoid, gastrointestinal (e.g., colon), genitourinary tract (e.g., renal, urothelial cells), prostate and pharynx. Adenocarcinomas include malignancies such as most colon cancers, rectal cancer, renal-cell carcinoma, liver cancer, non-small cell carcinoma of the lung, cancer of the small intestine and cancer of the esophagus. In one embodiment, the cancer is a melanoma, e.g., an advanced stage melanoma. Metastatic lesions of the aforementioned cancers can also be treated or prevented using the methods and compositions as described herein. Examples of other cancers that can be treated include bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin Disease, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi’s sarcoma, epidermoid cancer, squamous cell cancer, T-cell lymphoma, environmentally induced cancers including those induced by asbestos, and combinations of the cancers. Treatment of metastatic cancers, e.g., metastatic cancers that express PD-L1 (Iwai et al. (2005) Int. Immunol. 17:133-144) can be effected using the antibody molecules described herein.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder, such as cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment). In some embodiments, treatment of cancer includes decreasing tumor volume, decreasing the number of cancer cells, inhibiting cancer metastases, increasing life expectancy, decreasing cancer cell proliferation, decreasing cancer cell survival, or amelioration of various physiological symptoms associated with the cancerous condition.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount. In the context of lower tumor burden, the prophylactically effective amount in some aspects will be higher than the therapeutically effective amount.

In accordance with various embodiments described herein, the present disclosure provides engineered cells and compositions/formulations containing the engineered cells. The present disclosure also provides methods or processes for manufacturing the engineered cells, which may be useful for treating patients with a pathological disease or condition.

Further, in accordance with various embodiments described herein, the present disclosure provides a recombinant vector comprising a nucleic acid construct suitable for genetically modifying a cell, which may be used for treatment of pathological disease or condition.

Furthermore, in accordance with various embodiments described herein, the present disclosure provides an engineered cell comprising a nucleic acid construct suitable for genetically modifying a cell, which may be used for treatment of pathological disease or condition, wherein the nucleic acid encodes: (a) a genetically engineered antigen receptor that specifically binds to an antigen; and (b) an inhibitory protein that reduces, or is capable of effecting reduction of, expression of a tumor target. In various embodiments, the cell expresses the genetically engineered antigen receptor and the inhibitory protein. In various embodiments, the inhibitory protein is constitutively expressed.

Among the diseases, conditions, and disorders for treatment with the provided cells, compositions, methods and uses are tumors, including solid tumors, hematologic malignancies, and melanomas, and infectious diseases, such as infection with a virus or other pathogen, e.g., HPV, HIV, HCV, HBV, EBV, HTLV-1, CMV, adenovirus, BK polyomarvirus, HHV-8, MCV or other pathogens, and parasitic disease. In some embodiments, the disease or condition is a tumor, cancer, malignancy, neoplasm, or other proliferative disease or disorder. Such diseases include but are not limited to leukemia, lymphoma, e.g., chronic lymphocytic leukemia (CLL), acute- lymphoblastic leukemia (ALL), non-Hodgkin’s lymphoma, acute myeloid leukemia, multiple myeloma, refractory follicular lymphoma, mantle cell lymphoma, indolent B cell lymphoma, B cell malignancies, cancers of the uterine cervix, colon, lung, liver, breast, prostate, ovarian, skin, melanoma, bone, and brain cancer, ovarian cancer, epithelial cancers, renal cell carcinoma, pancreatic adenocarcinoma, Hodgkin lymphoma, cervical carcinoma, colorectal cancer, glioblastoma, neuroblastoma, Ewing sarcoma, medulloblastoma, osteosarcoma, synovial sarcoma, and/or mesothelioma.

### T Cell Receptors and Binding Molecules

T cells are a type of lymphocyte which typically develops in the thymus gland and plays a central role in the immune response. It plays an important role in the “adaptive immune response.” T cells can be distinguished from other lymphocytes by the presence of a T-cell receptor on the cell surface. Differentiated T cells have an important role in controlling the immune response. CD8+ T cells, also known as “killer cells”, are cytotoxic. Once they recognize a target cell, they are able to directly kill the target cell (e.g., virus-infected cells or cancer cells). CD8+ T cells can also produce cytokines and recruit other cells (e.g., macrophages and natural killer (NK) cells) to mount an immune response. CD4+ T cells, also known as “helper cells”, can indirectly kill target cells, e.g., by facilitating maturation of B cells into plasma cells and memory B cells, and activation of cytotoxic T cells and macrophages. Helper T cells become activated when they are presented with peptide antigens by MHC class II molecules, which are expressed on the surface of antigen-presenting cells (APCs). Once activated, they divide rapidly and secrete cytokines that regulate or assist the immune response. Regulatory T cells are important for tolerance, thereby preventing or inhibiting autoimmune response. The major role of regulatory T cells is to shut down T cell-mediated immunity toward the end of an immune reaction and to suppress autoreactive T cells that escaped the process of negative selection in the thymus.

T cells play an important role in cancer immunity where antigens from the cancer cells are taken up and presented on the cell surface of special immune cells called antigen-presenting cells (APCs) so that other immune cells can recognize the antigens of interest. In the lymph nodes, the APCs activate the T-cells and activate them to recognize the tumor cells. The activated T-cells can then travel via the blood vessels to reach the tumor, infiltrate it, recognize the cancer cells and kill them.

The activation of T cells requires T cell receptors. A “T cell receptor” or “TCR” is a molecule that contains a variable a (or alpha) and b (or beta) chains (also known as TCRα and TCRβ, respectively) or a variable g (or gamma) and d (or delta) chains (also known as TCRγ and TCRδ, respectively), or antigen-binding portions thereof, and which is capable of specifically binding to an antigen, e.g., a peptide antigen or peptide epitope bound to an major histocompatibility complex (MHC) molecule.

The present disclosure provides a T cell receptor (TCR) or antigen-binding fragment thereof, and binding molecules derived from TCR. In some embodiments, the TCR is in the αβ form. TCRs that exist in αβ and γδ forms are generally structurally similar, but T cells expressing them may have distinct anatomical locations or functions. Generally, a TCR is found on the surface of T cells (or T lymphocytes) where it is generally responsible for recognizing antigens, such as peptides bound to major histocompatibility complex (MHC) molecules.

In some embodiments, the TCR is an intact or full-length TCR, such as a TCR containing the a chain and b chain. In some embodiments, the TCR is an antigen-binding portion that is less than a full- length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable a (Va or Vα) chain and variable b (Vb or Vβ) chain of a TCR, or antigen -binding fragments thereof sufficient to form a binding site for binding to a specific MHC-peptide complex.

The variable domains of the TCR contain complementarity determining regions (CDRs), which generally are the primary contributors to antigen recognition and binding capabilities and specificity of the peptide, MHC and/or MHC-peptide complex. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs. In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some cases, CDR1 of the beta chain can interact with the C-terminal part of the peptide. In some contexts, CDR2 contributes most strongly to or is the primary CDR responsible for the interaction with or recognition of the MHC portion of the MHC-peptide complex.

The a-chain and/or b-chain of a TCR also can contain a constant domain, a transmembrane domain and/or a short cytoplasmic tail. In some aspects, each chain (e.g. alpha or beta) of the TCR can possess one N-terminal immunoglobulin variable domain, one immunoglobulin constant domain, a transmembrane region, and a short cytoplasmic tail at the C-terminal end. In some embodiments, a TCR, for example via the cytoplasmic tail, is associated with invariant proteins of the CD3 complex involved in mediating signal transduction. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g. CD3y, CD3δ, CD3e and CD3z chains) contain one or more immunoreceptor tyrosine-based activation motif or ITAM and generally are involved in the signaling capacity of the TCR complex.

The exact locus of a domain or region can vary depending on the particular structural or homology modeling or other features used to describe a particular domain. In some cases, the specific domain (e.g. variable or constant) can be several amino acids (such as one, two, three or four) longer or shorter. In some aspects, residues of a TCR are known or can be identified according to the International Immunogenetics Information System (IMGT) numbering system (see e.g. www.imgt.org; Lefranc et al. “IMGT unique numbering for immunoglobulin and T cell receptor variable domains and Ig superfamily V-like domains.” Developmental & Comparative Immunology 27.1 (2003): 55-77.). The structures and variations of TCR are known in the art, and are described, e.g., in WO 2019 /195486, which is incorporated herein by reference in its entirety.

In some embodiments, the a chain and b chain of a TCR each further contain a constant domain. In some embodiments, the a chain constant domain (Ca) and b chain constant domain (Cb) individually are mammalian, such as is a human or a non-human constant domain (e.g., a mouse constant domain). In some embodiments, the constant domain is adjacent to the cell membrane. For example, in some cases, the extracellular portion of the TCR formed by the two chains contains two membrane-proximal constant domains, and two membrane-distal variable domains, which variable domains each contain CDRs.

In some aspects, TCRs as descried herein can contain a human constant region, such as an alpha chain containing a human Ca region and a beta chain containing a human Cb regin. In some embodiments, the TCRs are fully human. In some embodiments, the expression and/or activity of TCRs, such as when expressed in human cells, e.g. human T cells, such as primary human T cells, are not impacted by or are not substantially impacted by the presence of an endogenous human TCR.

In some embodiments, the engineered TCRs are expressed at similar or improved levels on the cell surface, exhibit the similar or greater functional activity (e.g. cytolytic activity) and/or exhibit similar or greater anti-tumor activity, when expressed by human cells that contain or express an endogenous human TCR, such as human T cells, as compared to the level of expression, function activity and/or anti-tumor activity of the same TCR in similar human cells but in which expression of the endogenous TCR has been reduced or eliminated. In some examples an engineered TCR as described herein, when expressed in human T cells, is expressed on the cell surface at a level that is at least or at least about 80%, 85%, 90%, 95%, 100%, 105%, 110%, 115% or 120% of the level of expression of the same TCR when expressed in similar human T cells but in which expression of the endogenous TCR has been reduced or eliminated.

In some embodiments, each of the Ca and Cb domains is human. In some embodiments, the Ca is encoded by the TRAC gene (IMGT nomenclature) or is a variant thereof. In some embodiments, the variant of a Ca contains replacement of at least one non-native cysteine.

In some embodiments, the TCR can be a heterodimer of two chains a and b that are linked, such as by a disulfide bond or disulfide bonds. In some embodiments, the constant domain of the TCR can contain short connecting sequences in which a cysteine residue forms a disulfide bond, thereby linking the two chains of the TCR. In some embodiments, a TCR can have an additional cysteine residue in each of the a and b chains, such that the TCR contains two disulfide bonds in the constant domains. In some embodiments, each of the constant and variable domains contains disulfide bonds formed by cysteine residues.

In some embodiments, the native disulfide bonds are not present. In some embodiments, the one or more of the native cysteines (e.g. in the constant domain of the a chain and b chain) that form a native interchain disulfide bond are substituted to another residue, such as to a serine or alanine. In some embodiments, an introduced disulfide bond can be formed by mutating non cysteine residues on the alpha and beta chains, such as in the constant domain of the a chain and b chain, to cysteine. Opposing cysteines in the TCR a and b chains provide a disulfide bond that links the constant regions of TCR a and b chains of the substituted TCR to one another and which is not present in a TCR comprising the unsubstituted constant region in which the native disulfide bonds are present, such as unsubstituted native human constant region or the unsubstituted native mouse constant region. In some embodiments, the presence of non-native cysteine residues (e.g. resulting in one or more non-native disulfide bonds) in a recombinant TCR can favor production of the desired recombinant TCR in a cell in which it is introduced over expression of a mismatched TCR pair containing a native TCR chain.

In some embodiments, the TCR comprises CDRs, Va and/or Vb and constant region sequences as described herein.

In some embodiments, the TCR is a dimeric TCR (dTCR). In some embodiments a dTCR contains a first polypeptide wherein a sequence corresponding to a provided TCR a chain variable region sequence is fused to the N terminus of a sequence corresponding to a TCR a chain constant region extracellular sequence, and a second polypeptide wherein a sequence corresponding to a provided TCR b chain variable region sequence is fused to the N terminus a sequence corresponding to a TCR b chain constant region extracellular sequence, the first and second polypeptides being linked by a disulfide bond.

In some embodiments, a TCR can be cell-bound or in soluble form. In some embodiments, the TCR is in cell-bound form expressed on the surface of a cell.

In some embodiments, the TCR is a single chain TCR (scTCR). The scTCR is a single amino acid strand containing an a chain and a b chain that is able to bind to MHC-peptide complexes. Typically, a scTCR can be generated using methods known to those of skill in the art. These methods are described e.g., in WO 96/13593, WO 96/18105, WO99/18129, WO 04/033685, WO2006/037960, WO2011/044186; WO 2019 /195486; U.S. Pat. No. 7,569,664; each of which is incorporated herein by reference in its entirety.

The TCR, antigen binding fragments thereof, and TCR-derived binding molecules can bind or recognize a peptide epitope associated with an antigen of interest (e.g., a cancer antigen). In some embodiments, the antigen can be a peptide epitope expressed on the surface of a cancer cell and/or a cell infected with a virus, e.g., EBV. In some embodiments, the antigen is presented in the context of an MHC molecule. Such binding molecules include e.g., T cell receptors (TCRs) and antigen-binding fragments thereof, antibodies and antigen binding fragments thereof, and TCR-like CAR. They exhibit antigenic specificity for binding or recognizing such peptide epitopes. In some aspects, engineered cells that express a provided binding molecule, e.g. a TCR or antigen-binding fragment, exhibit cytotoxic activity against target cells expressing the peptide epitope, such as cancer cells or cells that are infected with EBV.

In some aspects, the TCR, antigen binding fragments thereof, and TCR-derived binding molecules recognize or bind to epitopes in the context of an MHC molecule, such as an MHC Class I molecule or an MHC class II molecule. Both MHC Class I molecules or MHC class II molecules are human leukocyte antigens (HLA). They play an important component of adaptive immune system. The HLA expression is controlled by genes located on chromosome 6. It encodes cell surface molecules specialized to present antigenic peptides to the T-cell receptor on T cells.

In some embodiments, the TCR, antigen binding fragments thereof, and TCR-derived binding molecules recognize or bind to epitopes in the context of an MHC Class I molecule. The MHC Class I molecule is a human leukocyte antigen (HLA)-A2 molecule, including any one or more subtypes thereof, e.g. HLA-A*0201, *0202, *0203, *0206, or *0207. The human leukocyte antigen A2 (HLA-A2) is among the most common human serotypes. In some cases, there can be differences in the frequency of subtypes between different populations. For example, more than 95% of the HLA-A2 positive Caucasian population is HLA-A*0201, whereas in the Chinese population the frequency has been reported to be approximately 23% for HLA-A*0201, 45% for HLA-A*0207, 8% for HLA-A*0206 and 23% for HLA-A*0203. In some embodiments, the MHC molecule is HLA-A*0201. In some embodiments, the present disclosure provides TCR or antigen-binding fragment thereof that bind an EBV LMP2/HLA-A2 complex, an EBV LMP2/HLA-A11 complex, or an EBV LMP2/HLA-A24 complex. In some embodiments, the present disclosure provides TCR or antigen-binding fragment thereof that bind an EBV LMP2 peptide FLYALALLL (SEQ ID NO: 139)/HLA-A2 complex, an EBV LMP2 peptide CLGGLLTMV (SEQ ID NO: 167)/HLA-A2 complex, an EBV LMP2 peptide SLGGLLTMV (SEQ ID NO: 168)/HLA-A2 complex, an EBV LMP2 peptide SSCSSCPLSK (SEQ ID NO: 140)/HLA-A11 complex, an EBV LMP2 peptide IYVLVMLVL (SEQ ID NO: 141)/HLA-A24 complex, or an EBV LMP2 peptide TYGPVFMCL (SEQ ID NO: 142)/HLA-A24 complex.

In some embodiments, the binding molecule, e.g., TCR or antigen-binding fragment thereof or TCR-derived binding molecule, is isolated or purified, or is recombinant. In some aspects, the binding molecule, e.g., TCR or antigen-binding fragment thereof or TCR-derived binding molecule, is fully human. In some embodiments, the binding molecule is monoclonal. In some aspects, the binding molecule is a single chain. In other embodiments, the binding molecule contains two chains. In some embodiments, the binding molecule, e.g., TCR, antigen-binding fragment thereof or TCR-derived binding molecule, is expressed on the surface of a cell.

The TRA locus contains V and J gene segments (TRAV and TRAJ gene segments) and the TRB locus contains V, D, and J gene segments (TRBV, TRBD, and TRBJ gene segments). The human TRA locus at 14q11.2 spans around 1000 kb. It consists of 54 TRAV genes belonging to 41 subgroups, 61 TRAJ segments localized on 71 kb, and a unique TRAC gene. The most 5′ TRAV genes occupy the most centromeric position, whereas the TRAC genes, 3′ of the locus, is the most telomeric gene in the TRA locus. The organization of the TRAJ segments on a large area is quite unusual and has not been observed in the other immunoglobulin or T cell receptor loci. Moreover, the TRD locus is nestled in the TRA locus between the TRAV and TRAJ segments. V-J-rearrangements in the TRA locus therefore result in deletion of the TRD genes localized on the same chromosome. That deletion occurs in two steps that is a deletion of the TRD genes, involving specific sequences located upstream from TRDC (sequence pseudo J alpha) would take place before the TRAV-J rearrangement. The genomic TRA repertoire comprises 45-47 functional TRAV genes belonging to 33-35 subgroups, 50 functional TRAJ segments, and the unique TRAC gene. Among the variable genes are included five genes designated as TRAV/DV which belong to five different subgroups and which have been found rearranged either to TRAJ or to TRDD segments and can therefore be used in the synthesis of alpha or delta chains. The total number of human TRA genes per haploid genome is 116 of which 96 to 98 genes are functional. Enhancer sequences have been characterized 4.5 kb 3′ from TRAC. A list of TRAV genes are shown in Table 1 and a list of TRVJ genes are shown in Table 2.

TABLE 1

List of TRAV genes on human chromosome 14

| Gene names | Order | Gene names | Order | Gene names | Order |
|---|---|---|---|---|---|
| TRAV1-1 | 1 | TRAV12-1 | 21 | TRAV27 | 41 |
| TRAV1-2 | 2 | TRAV12-2 | 22 | TRAV28 | 42 |
| TRAV2 | 3 | TRAV12-3 | 23 | TRAV29/DV5 | 43 |
| TRAV3 | 4 | TRAV13-1 | 24 | TRAV30 | 44 |
| TRAV4 | 5 | TRAV13-2 | 25 | TRAV31 | 45 |
| TRAV5 | 6 | TRAV14/DV4 | 26 | TRAV32 | 46 |
| TRAV6 | 7 | TRAV14-1 | 27 | TRAV33 | 47 |
| TRAV7 | 8 | TRAV15 | 28 | TRAV34 | 48 |
| TRAV8-1 | 9 | TRAV16 | 29 | TRAV35 | 49 |
| TRAV8-2 | 10 | TRAV17 | 30 | TRAV36/DV7 | 50 |
| TRAV8-3 | 11 | TRAV18 | 31 | TRAV37 | 51 |
| TRAV8-4 | 12 | TRAV19 | 32 | TRAV38-1 | 52 |
| TRAV8-5 | 13 | TRAV20 | 33 | TRAV38-2/DV8 | 53 |
| TRAV8-6 | 14 | TRAV21 | 34 | TRAV39 | 54 |
| TRAV8-6-1 | 15 | TRAV22 | 35 | TRAV40 | 55 |
| TRAV8-7 | 16 | TRAV23/DV6 | 36 | TRAV41 | 56 |
| TRAV9-1 | 17 | TRAV24 | 37 | TRAV46 | 57 |
| TRAV9-2 | 18 | TRAV25 | 38 | TRAVA | 58 |
| TRAV10 | 19 | TRAV26-1 | 39 | TRAVB | 59 |
| TRAV11 | 20 | TRAV26-2 | 40 | TRAVC | 60 |

TABLE 2

List of TRAJ genes on human chromosome 14

| Gene names | Order | Gene names | Order | Gene names | Order | Gene names | Order |
|---|---|---|---|---|---|---|---|
| TRAJ1 | 1 | TRAJ21 | 21 | TRAJ41 | 41 | TRAJ61 | 61 |
| TRAJ2 | 2 | TRAJ22 | 22 | TRAJ42 | 42 | | |
| TRAJ3 | 3 | TRAJ23 | 23 | TRAJ43 | 43 | | |
| TRAJ4 | 4 | TRAJ24 | 24 | TRAJ44 | 44 | | |
| TRAJ5 | 5 | TRAJ25 | 25 | TRAJ45 | 45 | | |
| TRAJ6 | 6 | TRAJ26 | 26 | TRAJ46 | 46 | | |
| TRAJ7 | 7 | TRAJ27 | 27 | TRAJ47 | 47 | | |
| TRAJ8 | 8 | TRAJ28 | 28 | TRAJ48 | 48 | | |
| TRAJ9 | 9 | TRAJ29 | 29 | TRAJ49 | 49 | | |
| TRAJ10 | 10 | TRAJ30 | 30 | TRAJ50 | 50 | | |
| TRAJ11 | 11 | TRAJ31 | 31 | TRAJ51 | 51 | | |
| TRAJ12 | 12 | TRAJ32 | 32 | TRAJ52 | 52 | | |
| TRAJ13 | 13 | TRAJ33 | 33 | TRAJ53 | 53 | | |
| TRAJ14 | 14 | TRAJ34 | 34 | TRAJ54 | 54 | | |
| TRAJ15 | 15 | TRAJ35 | 35 | TRAJ55 | 55 | | |
| TRAJ16 | 16 | TRAJ36 | 36 | TRAJ56 | 56 | | |
| TRAJ17 | 17 | TRAJ37 | 37 | TRAJ57 | 57 | | |
| TRAJ18 | 18 | TRAJ38 | 38 | TRAJ58 | 58 | | |
| TRAJ19 | 19 | TRAJ39 | 39 | TRAJ59 | 59 | | |
| TRAJ20 | 20 | TRAJ40 | 40 | TRAJ60 | 60 | | |

The human TRB locus at 7q35 spans 620 kb. It consists of 64-67 TRBV genes belonging to 32 subgroups. Except for TRBV30, localized downstream of the TRBC2 gene, in inverted orientation of transcription, all the other TRBV genes are located upstream of a duplicated D-J-C-cluster, which comprises, for the first part one TRBD, six TRBJ, and the TRBC1 gene, and for the second part, one TRBD, eight TRBJ, and the TRBC2 gene. The most 5′ TRBV genes occupy the most centromeric position, whereas the TRBV30 gene, 3′ of the locus, is the most telomeric gene in the TRB locus. The repertoire consists of 39-46 functional TRBV genes belonging to 21-23 subgroups, the two TRBD, thirteen TRBJ (6 from the first cluster and 7 from the second cluster), and the two TRBC genes. Six TRBV orphons have been localized on chromosome 9 at 9p21. Enhancer sequences have been characterized 5.5 kb 3′ from TRBC2. Lists of TRBV, TRBD and TRBJ genes are shown in Table 3, Table 4 and Table 5, respectively.

TABLE 3

List of TRBV genes on human chromosome 7

| Gene names | Order | Gene names | Order | Gene names | Order | Gene names | Order |
|---|---|---|---|---|---|---|---|
| TRBV1 | 1 | TRBV6-6 | 21 | TRBV11-2 | 41 | TRBV26 | 61 |
| TRBV2 | 2 | TRBV6-7 | 22 | TRBV11-3 | 42 | TRBV27 | 62 |
| TRBV3-1 | 3 | TRBV6-8 | 23 | TRBV12-1 | 43 | TRBV28 | 63 |
| TRBV3-2 | 4 | TRBV6-9 | 24 | TRBV12-2 | 44 | TRBV29-1 | 64 |
| TRBV4-1 | 5 | TRBV7-1 | 25 | TRBV12-3 | 45 | TRBV30 | 65 |
| TRBV4-2 | 6 | TRBV7-2 | 26 | TRBV12-4 | 46 | TRBVA | 66 |
| TRBV4-3 | 7 | TRBV7-3 | 27 | TRBV12-5 | 47 | TRBVB | 67 |
| TRBV5-1 | 8 | TRBV7-4 | 28 | TRBV13 | 48 | TRBVC | 68 |
| TRBV5-2 | 9 | TRBV7-5 | 29 | TRBV14 | 49 | | |
| TRBV5-3 | 10 | TRBV7-6 | 30 | TRBV15 | 50 | | |
| TRBV5-4 | 11 | TRBV7-7 | 31 | TRBV16 | 51 | | |
| TRBV5-5 | 12 | TRBV7-8 | 32 | TRBV17 | 52 | | |
| TRBV5-6 | 13 | TRBV7-9 | 33 | TRBV18 | 53 | | |
| TRBV5-7 | 14 | TRBV8-1 | 34 | TRBV19 | 54 | | |
| TRBV5-8 | 15 | TRBV8-2 | 35 | TRBV20-1 | 55 | | |
| TRBV6-1 | 16 | TRBV9 | 36 | TRBV21-1 | 56 | | |
| TRBV6-2 | 17 | TRBV10-1 | 37 | TRBV22-1 | 57 | | |
| TRBV6-3 | 18 | TRBV10-2 | 38 | TRBV23-1 | 58 | | |
| TRBV6-4 | 19 | TRBV10-3 | 39 | TRBV24-1 | 59 | | |
| TRBV6-5 | 20 | TRBV11-1 | 40 | TRBV25-1 | 60 | | |

TABLE 4

List of TRBD genes on human chromosome 7

| Gene names | Order |
|---|---|
| TRBD1 | 1 |
| TRBD2 | 2 |

TABLE 5

List of TRBJ genes on human chromosome 7

| Gene names | Order |
|---|---|
| TRBJ1-1 | 1 |
| TRBJ1-2 | 2 |
| TRBJ1-3 | 3 |
| TRBJ1-4 | 4 |
| TRBJ1-5 | 5 |
| TRBJ1-6 | 6 |
| TRBJ2-1 | 7 |
| TRBJ2-2 | 8 |
| TRBJ2-2P | 9 |
| TRBJ2-3 | 10 |
| TRBJ2-4 | 11 |
| TRBJ2-5 | 12 |
| TRBJ2-6 | 13 |
| TRBJ2-7 | 14 |

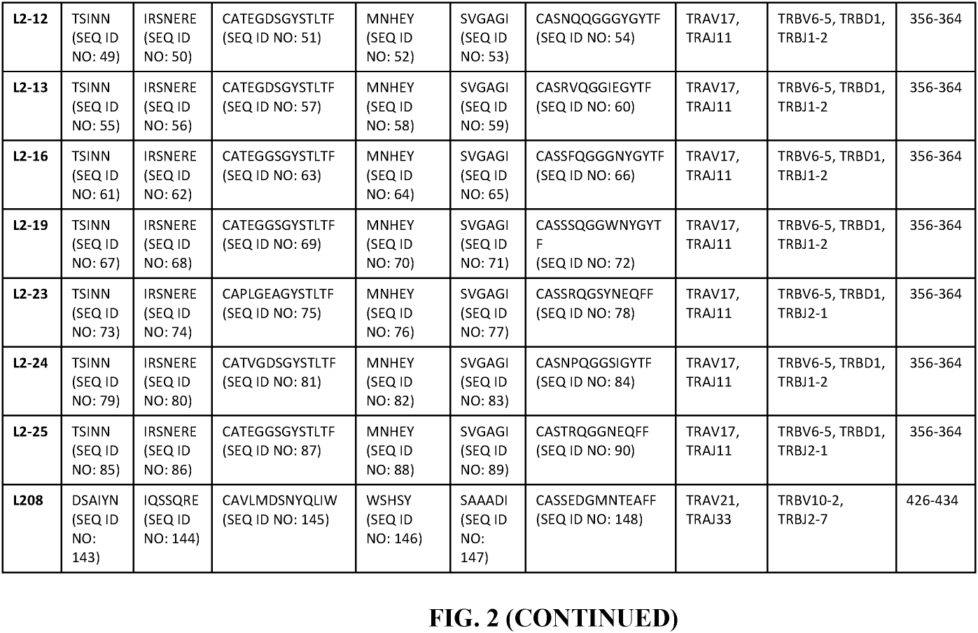

In some embodiments, the TCR, TCR derived binding molecules, or antigen-binding fragment thereof, comprising an alpha chain comprising a variable alpha (Va) region and a beta chain comprising a variable beta (Vb) region, wherein the Va region can have complementarity determining regions (CDRs) 1, 2, 3, wherein the CDR1 region comprises or consists of an amino acid sequence that is at least 80%, 85%, 90%, or 95% identical to a selected Va CDR1 amino acid sequence, the CDR2 region comprises or consists of an amino acid sequence that is at least 80%, 85%, 90%, or 95% identical to a selected Va CDR2 amino acid sequence, and the CDR3 region comprises or consists of an amino acid sequence that is at least 80%, 85%, 90%, or 95% identical to a selected Va CDR3 amino acid sequence, and a variable beta (Vb) region comprising CDRs 1, 2, 3, wherein the CDR1 region comprises or consists of an amino acid sequence that is at least 80%, 85%, 90%, or 95% identical to a selected Vb CDR1 amino acid sequence, the CDR2 region comprises or consists of an amino acid sequence that is at least 80%, 85%, 90%, or 95% identical to a selected Vb CDR2 amino acid sequence, and the CDR3 region comprises or consists of an amino acid sequence that is at least 80%, 85%, 90%, or 95% identical to a selected Vb CDR3 amino acid sequence. The selected Va CDRs 1, 2, 3 amino acid sequences and the selected Vb CDRs, 1, 2, 3 amino acid sequences are shown in FIGS. 2-4.

In some embodiments, the Va region comprises the amino acid sequence set forth in any of SEQ ID NOs: 170, 172, 174, 176, 178, 180, 182, 184, 186, 188, 190, 192, 194, 196, 198, 200, 202, 204, 206, 208, 210, 212, 214, 216, 218, 220, or 222, or an amino acid sequence that has at least 80%, 85%, 90%, 91%, 92%, 93%, 94%, 95%, 96%, 97%, 98%, or 99% sequence identity thereto. In some embodiments, the Vb region comprises the amino acid sequence set forth in any of SEQ ID NOs: 171, 173, 175, 177, 179, 181, 183, 185, 187, 189, 191, 193, 195, 197, 199, 201, 203, 205, 207, 209, 211, 213, 215, 217, 219, 221, or 223, or an amino acid sequence that has at least 80%, 85%, 90%, 91%, 92%, 93%, 94%, 95%, 96%, 97%, 98%, or 99% sequence identity thereto. In some embodiments, the Va region comprises one or more Va CDR sequences as described herein. In some embodiments, the Vb region comprises one or more Vb CDR sequences as described herein.

In some embodiments, provided herein is a TCR having a Va region that is at least 80%, 85%, 90%, or 95% identical SEQ ID NO: 170, and a Vb region that is at least 80%, 85%, 90%, or 95% identical SEQ ID NO: 171.

In some embodiments, provided herein is a TCR having a Va region that is at least 80%, 85%, 90%, or 95% identical SEQ ID NO: 172; and a Vb region that is at least 80%, 85%, 90%, or 95% identical SEQ ID NO: 173.

In some embodiments, provided herein is a TCR having a Va region that is at least 80%, 85%, 90%, or 95% identical SEQ ID NO: 174; and a Vb region that is at least 80%, 85%, 90%, or 95% identical SEQ ID NO: 175.

In some embodiments, provided herein is a TCR having a Va region that is at least 80%, 85%, 90%, or 95% identical SEQ ID NO: 176; and a Vb region that is at least 80%, 85%, 90%, or 95% identical SEQ ID NO: 177.

In some embodiments, provided herein is a TCR having a Va region that is at least 80%, 85%, 90%, or 95% identical SEQ ID NO: 178; and a Vb region that is at least 80%, 85%, 90%, or 95% identical SEQ ID NO: 179.

In some embodiments, provided herein is a TCR having a Va region that is at least 80%, 85%, 90%, or 95% identical SEQ ID NO: 180; and a Vb region that is at least 80%, 85%, 90%, or 95% identical SEQ ID NO: 181.

In some embodiments, provided herein is a TCR having a Va region that is at least 80%, 85%, 90%, or 95% identical SEQ ID NO: 182; and a Vb region that is at least 80%, 85%, 90%, or 95% identical SEQ ID NO: 183.

In some embodiments, provided herein is a TCR having a Va region that is at least 80%, 85%, 90%, or 95% identical SEQ ID NO: 184; and a Vb region that is at least 80%, 85%, 90%, or 95% identical SEQ ID NO: 185.

In some embodiments, provided herein is a TCR having a Va region that is at least 80%, 85%, 90%, or 95% identical SEQ ID NO: 186; and a Vb region that is at least 80%, 85%, 90%, or 95% identical SEQ ID NO: 187.

In some embodiments, provided herein is a TCR having a Va region that is at least 80%, 85%, 90%, or 95% identical SEQ ID NO: 188; and a Vb region that is at least 80%, 85%, 90%, or 95% identical SEQ ID NO: 189.

In some embodiments, provided herein is a TCR having a Va region that is at least 80%, 85%, 90%, or 95% identical SEQ ID NO: 190; and a Vb region that is at least 80%, 85%, 90%, or 95% identical SEQ ID NO: 191.

In some embodiments, provided herein is a TCR having a Va region that is at least 80%, 85%, 90%, or 95% identical SEQ ID NO: 192; and a Vb region that is at least 80%, 85%, 90%, or 95% identical SEQ ID NO: 193.

In some embodiments, provided herein is a TCR having a Va region that is at least 80%, 85%, 90%, or 95% identical SEQ ID NO: 194; and a Vb region that is at least 80%, 85%, 90%, or 95% identical SEQ ID NO: 195.

In some embodiments, provided herein is a TCR having a Va region that is at least 80%, 85%, 90%, or 95% identical SEQ ID NO: 196; and a Vb region that is at least 80%, 85%, 90%, or 95% identical SEQ ID NO: 197.

In some embodiments, provided herein is a TCR having a Va region that is at least 80%, 85%, 90%, or 95% identical SEQ ID NO: 198; and a Vb region that is at least 80%, 85%, 90%, or 95% identical SEQ ID NO: 199.

In some embodiments, provided herein is a TCR having a Va region that is at least 80%, 85%, 90%, or 95% identical SEQ ID NO: 200; and a Vb region that is at least 80%, 85%, 90%, or 95% identical SEQ ID NO: 201.

In some embodiments, provided herein is a TCR having a Va region that is at least 80%, 85%, 90%, or 95% identical SEQ ID NO: 202; and a Vb region that is at least 80%, 85%, 90%, or 95% identical SEQ ID NO: 203.

In some embodiments, provided herein is a TCR having a Va region that is at least 80%, 85%, 90%, or 95% identical SEQ ID NO: 204; and a Vb region that is at least 80%, 85%, 90%, or 95% identical SEQ ID NO: 205.

In some embodiments, provided herein is a TCR having a Va region that is at least 80%, 85%, 90%, or 95% identical SEQ ID NO: 206; and a Vb region that is at least 80%, 85%, 90%, or 95% identical SEQ ID NO: 207.

In some embodiments, provided herein is a TCR having a Va region that is at least 80%, 85%, 90%, or 95% identical SEQ ID NO: 208; and a Vb region that is at least 80%, 85%, 90%, or 95% identical SEQ ID NO: 209.

In some embodiments, provided herein is a TCR having a Va region that is at least 80%, 85%, 90%, or 95% identical SEQ ID NO: 210; and a Vb region that is at least 80%, 85%, 90%, or 95% identical SEQ ID NO: 211.

In some embodiments, provided herein is a TCR having a Va region that is at least 80%, 85%, 90%, or 95% identical SEQ ID NO: 212; and a Vb region that is at least 80%, 85%, 90%, or 95% identical SEQ ID NO: 213.

In some embodiments, provided herein is a TCR having a Va region that is at least 80%, 85%, 90%, or 95% identical SEQ ID NO: 214; and a Vb region that is at least 80%, 85%, 90%, or 95% identical SEQ ID NO: 215.

In some embodiments, provided herein is a TCR having a Va region that is at least 80%, 85%, 90%, or 95% identical SEQ ID NO: 216; and a Vb region that is at least 80%, 85%, 90%, or 95% identical SEQ ID NO: 217.

In some embodiments, provided herein is a TCR having a Va region that is at least 80%, 85%, 90%, or 95% identical SEQ ID NO: 218; and a Vb region that is at least 80%, 85%, 90%, or 95% identical SEQ ID NO: 219.

In some embodiments, provided herein is a TCR having a Va region that is at least 80%, 85%, 90%, or 95% identical SEQ ID NO: 220; and a Vb region that is at least 80%, 85%, 90%, or 95% identical SEQ ID NO: 221.

In some embodiments, provided herein is a TCR having a Va region that is at least 80%, 85%, 90%, or 95% identical SEQ ID NO: 222; and a Vb region that is at least 80%, 85%, 90%, or 95% identical SEQ ID NO: 223.

In some embodiments, the TCR, antigen-binding fragment thereof, or TCR-derived binding molecules described herein can contain a variable region (e.g., Va) containing one, two, or three of the CDRs described herein with zero, one or two amino acid insertions, deletions, or substitutions. In some embodiments, the a chain comprises one or more Va CDR sequences as described herein. In some embodiments, the b chain comprises one or more Vb CDR sequences as described herein.

In some embodiments, the TCR, antigen binding fragment thereof, or TCR-derived binding molecules described herein include an alpha chain comprising a variable alpha (Va) region and a beta chain comprising a variable beta (Vb) region. In some embodiments, the Va region can include a complementarity determining region 1 (CDR1), a complementarity determining region 2 (CDR2), and a complementarity determining region 3 (CDR3). In some embodiments, the CDR3 of the Va region comprises an amino acid sequence including SGY (serine-glycine-tyrosine). In some embodiments, the CDR3 of the Va region comprises an amino acid sequence including YST (tyrosine-serine-threonine). In some embodiments, the CDR3 of the Va region comprises an amino acid sequence X₁GX₂SGYSTL, in which the X₁ is a E (glutamic acid), T (threonine), Q (glutamine), V (valine), or N (asparagine); and the X₂ is a D (aspartic acid), G (glycine), N (asparagine), or E (glutamic acid). In some embodiments, the Vb region comprises a CDR1, a CDR2, and a CDR3. In some embodiments, the CDR3 of the Vb region comprises an amino acid sequence including QGG (glutamine-glycine-glycine). In some embodiments, the CDR3 of the Vb region comprises an amino acid sequence including TQGG (threonine-glutamine-glycine-glycine). In some embodiments, the CDR3 of the Vb region comprises an amino acid sequence X₃X₄QGGX₅X₆X₇X₈, in which the X₃ is a S (serine), T (threonine), N (asparagine), or R (arginine); the X₄ is a T (threonine), R (arginine), Y (tyrosine), G (glycine), V (valine), Q (glutamine), F (phenylalanine), S (serine), or P (proline); the X₅ is a N (asparagine), G (glycine), H (histidine), T (threonine), S (serine), A (alanine), I (isoleucine), or W (tryptophan); the X₆ is a Y (tyrosine), N (asparagine), D (aspartic acid), E (glutamic acid), R (arginine), or I (isoleucine); the X₇ is a G (glycine), Q (glutamine), N (asparagine), Y (tyrosine); and the X₈ is a Y (tyrosine), F (phenylalanine), or G (glycine).

In some embodiments, the TCR, antigen binding fragment thereof, or TCR-derived binding molecules described herein include an alpha chain comprising a variable alpha (Va) region and a beta chain comprising a variable beta (Vb) region. In some embodiments, the Va region is encoded by a sequence from rearrangement of a human TRAV gene segment and a human TRAJ gene segment, and the Vb region is encoded by a sequence from rearrangement of a human TRBV gene segment, a human TRBD gene segment, and a human TRBJ gene segment. In some embodiments, the TRAV gene segment is TRAV17; the TRAJ gene segment is TRAJ11; the TRBV gene segment is TRBV6-5; the TRBD gene segment is TRBD1 or TRBD2; and the TRBJ gene segment is TRBJ2-1 or TRBJ1-2. In some embodiments, the TRAV gene segment is TRAV21; the TRAJ gene segment is TRAJ33; the TRBV gene segment is TRBV10-2; the TRBD gene segment is TRBD1; and the TRBJ gene segment is TRBJ2-7. In some embodiments, the TRAV gene segment is TRAV4, TRAV25, TRAV22, or TRAV6; the TRAJ gene segment is TRAJ23, TRAJ47, TRAJ29, TRAJ43, or TRAJ11; the TRBV gene segment is TRBV12-4, TRBV12-3, TRBV11-2, TRBV4-1, or TRBV11-2; the TRBD gene segment is TRBD2; and the TRBJ gene segment is TRBJ1-1, TRBJ2-1, TRBJ2-5, or TRBJ2-7. In some embodiments, the TRAV gene segment is TRAV12-1; the TRAJ gene segment is TRAJ21; the TRBV gene segment is TRBV20-1; the TRBD gene segment is TRBD1; and the TRBJ gene segment is TRBJ2-5 or TRBJ2-3. In some embodiments, the TRAV gene segment is TRAV25, TRAV12-3, or TRAV21; the TRAJ gene segment is TRAJ16, TRAJ3, or TRAJ35; the TRBV gene segment is TRBV6-6, TRBV24-1, or TRBV30; the TRBD gene segment is TRBD1 or TRBD2; and the TRBJ gene segment is TRBJ1-1, TRBJ2-5, TRBJ2-3, or TRBJ2-7.

In some embodiments, the CDRs of the Va region are encoded by sequences from a human TRAV gene segment and a human TRAJ gene segment, wherein the TRAV gene segment is TRAV17 and the TRAJ gene segment is TRAJ11. In some embodiments, the CDRs of the Vb region are encoded by sequences from a human TRBV gene segment, a human TRBD gene segment, and a human TRBJ gene segment, wherein the TRBV gene segment is TRBV6-5; the TRBD gene segment is TRBD1 or TRBD2; and the TRBJ gene segment is TRBJ2-1 or TRBJ1-2.

In some embodiments, the CDRs of the Va region are encoded by sequences from a human TRAV gene segment and a human TRAJ gene segment, wherein the TRAV gene segment is TRAV21 and the TRAJ gene segment is TRAJ33. In some embodiments, the CDRs of the Vb region are encoded by sequences from a human TRBV gene segment, a human TRBD gene segment, and a human TRBJ gene segment, wherein the TRBV gene segment is TRBV10-2; the TRBD gene segment is TRBD1; and the TRBJ gene segment is TRBJ2-7.

The present disclosure also provides an antibody or antigen-binding fragment thereof that contains any one or more of the CDRs as described above with respect to TCRs. In some embodiments, the antibody or antigen-binding fragment contains variable heavy and light chain containing a CDR1, a CDR2 and/or a CDR3 contained in the alpha chain and a CDR1, a CDR2 and/or a CDR3 contained in the beta chain.

The disclosure also provides nucleic acid comprising a polynucleotide encoding a polypeptide comprising a TCR a chain variable region, a TCR b chain variable region, an immunoglobulin heavy chain variable region or an immunoglobulin light chain variable region. The variable region comprises CDRs as shown in FIGS. 2-4. When the polypeptides are paired with corresponding polypeptide (e.g., a corresponding a chain variable region or a corresponding b chain variable region), the paired polypeptides bind to the antigen of interest (e.g., EBV LMP2).

In some embodiments, by binding to the antigen of interest, the TCR or antigen-binding fragment thereof, or TCR-derived binding molecules, can activate T cells (e.g., by activating TCR signaling pathway). In some embodiments, the activation can upregulate immune response, increase expression of cytokines (e.g., IFNy) and/or CD107a, promote T-cell proliferation and T cell mediated killing.

In some embodiments, the TCR or antigen-binding fragment thereof, or TCR-derived binding molecules as described herein can increase immune response, activity or number of T cells by at least 10%, 20%, 30%, 40%, 50%, 60%, 70%, 80%, 90%, 100%, 2 folds, 3 folds, 5 folds, 10 folds, or 20 folds. In some embodiments, the TCR or antigen-binding fragment thereof, or TCR-derived binding molecules, when the antigen of interest is present, can increase serum concentrations of IFN-y. In some embodiments, the activation can induce at least 10%, 20%, 30%, 40%, 50%, 60%, 70%, 80%, 90%, 1 fold, 2 folds, 5 folds, 10 folds, 100 folds, or 1000 folds increase of the serum concentrations of IFN-y. In some embodiments, the activation can induce at least 10%, 20%, 30%, 40%, 50%, 60%, 70%, 80%, 90%, 1 fold, 2 folds, 3 folds, 4 folds, or 5 folds increase of specific killing of target cells.

In some aspects, the provided recombinant TCRs include TCRs that are at least partially CD8-independent. In some aspects, the provided recombinant TCRs include TCRs that are at least partially CD8-dependent.

In some embodiments, the TCR or antigen-binding fragment thereof, or TCR-derived binding molecules as described herein specifically binds to EBV LMP2 epitope. In some embodiments, the epitope has a sequence selected from SEQ ID NOS: 139-142, 167, and 168. Binding affinities can be deduced from the quotient of the kinetic rate constants (KD=k_{off}/kₒₙ). In some embodiments, KD is less than 1 × 10⁻⁶ M, less than 1 × 10⁻⁷ M, less than 1 × 10⁻⁸ M, less than 1 × 10⁻⁹ M, or less than 1 × 10⁻¹⁰ M. In some embodiments, the KD is less than 50 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1 × 10⁻⁷ M, greater than 1 × 10⁻⁸ M, greater than 1 × 10⁻⁹ M, greater than 1 × 10⁻¹⁰ M, greater than 1 × 10⁻¹¹ M, or greater than 1 × 10⁻¹² M. General techniques for measuring the affinity of a binding molecule for an antigen include, e.g., ELISA, RIA, and surface plasmon resonance (SPR).

In some embodiments, the TCR or antigen-binding fragment thereof, or TCR-derived binding molecules have a relatively high expression efficiency. For example, the expression efficiency for the TCR or antigen-binding fragment thereof, or TCR-derived binding molecules described herein can be at least 10%, 20%, 30%, 40%, 50%, or 100% higher than an reference molecule (e.g., an endogenous TCR) under the same conditions.

In some embodiments, the binding molecule, e.g. TCR, does not exhibit cross-reactive or off-target binding, such as undesirable off-target binding, e.g. off-target binding to antigens present in healthy or normal tissues or cells.

### EBV Infection and Cancer

Epstein Barr Virus (EBV) was one of the first viruses to be identified as oncogenic. EBV is extremely effective in infecting B cells through its interaction with CD21 and MHC class II. EBV can also infect and be retained in epithelial cells. Virtually all adults in the world have been exposed to EBV. In the absence of immune compromise, initial exposure in childhood results in a self-limited illness controlled by a cellular immune response. The presence of an immune defense against Epstein Barr Virus (EBV) and EBV- associated disease is well known. The host’s generation of antigen specific T-cells against viral proteins is very effective against the virus. However, EBV can persist in epithelial or B cells without being completely eliminated. Any changes in the immune status of the host can lead to re-activation and depending on the degree of immune compromise, this re-activation can lead to malignancy.

EBV is involved in solid organ and hematopoietic cell transplantation (HSCT) where a decreased number or absence of T-cells may cause un-restricted proliferation of B-cells harboring EBV. Such uncontrolled expansion can lead to post transplant lymphoproliferative disease (PTLD), the most common post-transplant malignancy. The frequency and intensity of this syndrome varies within each patient and the effects of their immune suppression on their T-cell population. EBV is also involved in other malignancies. Several lines of research have implicated EBV in the pathogenesis of various epithelial and lymphoid malignancies. For example, it is well known that Hodgkin (Glaser, et al. “Epstein-Barr virus-associated Hodgkin’s disease: epidemiologic characteristics in international data.” International journal of cancer 70.4 (1997): 375-382) and non-Hodgkin Lymphomas are related to EBV. There is also a clear causal relationship between EBV and nasopharyngeal carcinoma (NPC; Raab-Traub “Nasopharyngeal carcinoma: an evolving role for the Epstein-Barr virus.” Epstein Barr Virus Volume 1. Springer, Cham, 2015. 339-363.). Tumor samples of patients with Hodgkin Lymphoma and NPC express EBV derived proteins including the latent membrane protein 2 (LMP2). Because these viral proteins are non-self and are also the main targets of the cellular immune response against EBV, they represent ideal targets for anticancer immunotherapy.

The list of LMP2(+) human malignancies associated with EBV includes Burkitt’s lymphoma, immunosuppressive lymphoma, diffuse large B-cell lymphoma, diffuse large B-cell lymphoma associated with chronic inflammation, lymphomatoid granulomatosis, plasmablastic lymphoma, primary effusion lymphoma, post-transplant lymphoproliferative disorder, nasopharyngeal carcinoma, gastric adenocarcinoma, lymphoepithelioma-associated carcinoma, and immunodeficiency-related leiomyosarcoma. These disorders are described e.g., in WO/2019/213416 A1; Thompson et al., “Epstein-Barr virus and cancer.” Clinical Cancer Research 10.3 (2004): 803-821, both of which are incorporated herein by reference in the entirety.

The EBV infection/transformation of resting B-cells produces Latent Lymphoblastoma Lines (LCL). LCLs present in latent replication and carry multiple copies of the viral genome as an episome. They express a number of viral gene products denominated latent proteins that vary according to latency stage. A total of ten latency proteins have been described: Six Epstein Virus Nuclear Antigens (EBNA 1, 2, 3A, 3B, 3C and LP), three Latent Membrane Proteins (LMP 1, 2A and 2B) and BARF1. Initial EBV infection activates B-cells and induces latency III when EBNA1, EBNA2, EBNA3, LMP1, LMP2 and BARF1 are expressed. These proteins are described e.g., in Bollard, et al., “T-cell therapy in the treatment of post-transplant lymphoproliferative disease.” Nature reviews Clinical oncology 9.9 (2012): 510, which is incorporated herein by reference in its entirety.

The present disclosure provides methods of treating EBV infection and/or EBV induced disease and disorders.

### Engineered Cells

The present disclosure provides engineered cells (e.g., T cells) that comprise TCR or antigen-binding fragment thereof, or other similar antigen-binding molecules as described herein. These engineered cells can be used to treat various disorders or disease as described herein (e.g., virus infection, cancers, virus-induced disorders).

In various embodiments, the cell that is engineered can be obtained from e.g., humans and non-human animals. In various embodiments, the cell that is engineered can be obtained from bacteria, fungi, humans, rats, mice, rabbits, monkeys, pig or any other species. Preferably, the cell is from humans, rats or mice. More preferably, the cell is obtained from humans. In various embodiments, the cell that is engineered is a blood cell. Preferably, the cell is a leukocyte (e.g., a T cell), lymphocyte or any other suitable blood cell type. In some embodiments, the cell is a peripheral blood cell. In some embodiments, the cell is a T cell, B cell or NK cell.

In some embodiments, the cell is a T cell. In some embodiments, the T cells can express a cell surface receptor that recognizes a specific antigenic moiety on the surface of a target cell. The cell surface receptor can be a wild type or recombinant T cell receptor (TCR), a chimeric antigen receptor (CAR), or any other surface receptor capable of recognizing an antigenic moiety that is associated with the target cell. T cells can be obtained by various methods known in the art, e.g., in vitro culture of T cells (e.g., tumor infiltrating lymphocytes) isolated from patients. TCR gene-modified T cells can be obtained by transducing T cells (e.g., isolated from the peripheral blood of patients), with a viral vector. In some embodiments, the T cell is a TCR gene-modified T cell. In some embodiments, the T cells are CD4+ T cells, CD8+ T cells, or regulatory T cells. In some embodiments, the T cells are T helper type 1 T cells and T helper type 2 T cells. In some embodiments, the T cell expressing this receptor is an αβ-T cell. In alternate embodiments, the T cell expressing this receptor is a γδ-T cell.

In some embodiments, the cell is an NK cell. In some embodiments, preparation of the engineered cells includes one or more culture and/or preparation steps. The cells for introduction of the binding molecule, e.g., TCR, can be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

In some embodiments, the cells are stem cells, such as multipotent and pluripotent stem cells, including induced pluripotent stem cells (iPSCs). The cells can be primary cells, such as those isolated directly from a subject and/or isolated from a subject and frozen. In some embodiments, the stem cells are cultured with additional differentiation factors to obtain desired cell types (e.g., T cells).

Different cell types can be obtained from appropriate isolation methods. The isolation methods include the separation of different cell types based on the expression or presence in the cell of one or more specific molecules, such as surface markers, e.g., surface proteins, intracellular markers, or nucleic acid. In some embodiments, any known method for separation based on such markers can be used. In some embodiments, the separation is affinity- or immunoaffinity-based separation. For example, the isolation in some aspects includes separation of cells and cell populations based on the cells’ expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

Also provided are methods, nucleic acids, compositions, and kits, for expressing the binding molecules, and for producing the genetically engineered cells expressing such binding molecules. The genetic engineering generally involves introduction of a nucleic acid encoding the therapeutic molecule, e.g. TCR, CAR, e.g. TCR-like CAR, polypeptides, fusion proteins, into the cell, such as by retroviral transduction, transfection, or transformation. In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical application.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors. In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), or spleen focus forming virus (SFFV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In some embodiments, the vector is a lentivirus vector. In some embodiments, recombinant nucleic acids are transferred into T cells via electroporation. In some embodiments, recombinant nucleic acids are transferred into T cells via transposition. Other methods of introducing and expressing genetic material in immune cells include calcium phosphate transfection, protoplast fusion, cationic liposome-mediated transfection; tungsten particle-facilitated microparticle bombardment and strontium phosphate DNA co-precipitation. Many of these methods are descried e.g., in WO2019195486, which is incorporated herein by reference in its entirety.

In some aspects, development of a humanized and/or fully human recombinant TCR presents technical challenges. For example, in some aspects, a humanized and/or a fully human recombinant TCR receptor, when engineered into a human T cell, may compete with endogenous TCR complexes and/or can form mispairings with endogenous TCRa and/or TCRb chains, which may, in certain aspects, reduce recombinant TCR signaling, activity, and/or expression, and ultimately result in reduced activity of the engineered cells. The engineered cell can be genetically modified. In some embodiments, the engineered cells can comprise a genetic disruption of a T cell receptor alpha constant (TRAC) gene and/or a T cell receptor beta constant (TRBC) gene. In some embodiments, the TRBC gene is one or both of a T cell receptor beta constant 1 (TRBCJ) or T cell receptor beta constant 2 (TRBC2) gene. In some embodiments, the engineered cells do not express endogenous TCR a chain and/or TRC b chain. In some other aspects, non-human constant domains are used, e.g., rodent (e.g., mouse) constant domains. The use of non-human constant domains can effectively reduce the likelihood of mispairing.

Also provided are populations of engineered cells, compositions containing such cells and/or enriched for such cells, such as in which cells expressing the binding molecule make up at least 15%, 20%, 25%, 30%, 35%, 40%, 50%, 60%, 70%, 80%, 90%, 91 %, 92%, 93%, 94%, 95%, 96%, 97%, 98%, 99%, or more percent of the total cells in the composition or cells of a certain type such as T cells, CD8+ or CD4+ cells.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

The present disclosure provides a recombinant vector comprising a nucleic acid construct suitable for genetically modifying a cell, which can be used for treatment of pathological disease or condition.

Any vector or vector type can be used to deliver genetic material to the cell. These vectors include but are not limited to plasmid vectors, viral vectors, bacterial artificial chromosomes (BACs), yeast artificial chromosomes (YACs), and human artificial chromosomes (HACs). Viral vectors can include but are not limited to recombinant retroviral vectors, recombinant lentiviral vectors, recombinant adenoviral vectors, foamy virus vectors, recombinant adeno-associated viral (AAV) vectors, hybrid vectors, and plasmid transposons (e.g., sleeping beauty transposon system, and PiggyBac transposon system) or integrase based vector systems. Other vectors that are known in the art can also be used in connection with the methods described herein.

In some embodiments, the vector is a viral vector. The viral vector can be grown in a culture medium specific for viral vector manufacturing. Any suitable growth media and/or supplements for growing viral vectors can be used in accordance with the embodiments described herein.

In some embodiments, the vector used is a recombinant retroviral vector. A retroviral vector is capable of directing the expression of a nucleic acid molecule of interest. A retrovirus is present in the RNA form in its viral capsule and forms a double-stranded DNA intermediate when it replicates in the host cell. Similarly, retroviral vectors are present in both RNA and double-stranded DNA forms. The retroviral vector also includes the DNA form which contains a recombinant DNA fragment and the RNA form containing a recombinant RNA fragment. The vectors can include at least one transcriptional promoter/enhancer, or other elements which control gene expression. Such vectors can also include a packaging signal, long terminal repeats (LTRs) or portion thereof, and positive and negative strand primer binding sites appropriate to the retrovirus used. Long terminal repeats (LTRs) are identical sequences of DNA that repeat many times (e.g., hundreds or thousands of times) found at either end of retrotransposons or proviral DNA formed by reverse transcription of retroviral RNA. They are used by viruses to insert their genetic material into the host genomes. Optionally, the vectors can also include a signal which directs polyadenylation, selectable markers such as Ampicillin resistance, Neomycin resistance, TK, hygromycin resistance, phleomycin resistance histidinol resistance, or DHFR, as well as one or more restriction sites and a translation termination sequence. For example, such vectors can include a 5′ LTR, a leading sequence, a tRNA binding site, a packaging signal, an origin of second strand DNA synthesis, and a 3′ LTR or a portion thereof. Additionally, retroviral vector used herein can also refers to the recombinant vectors created by removal of the retroviral gag, pol, and env genes and replaced with the gene of interest.

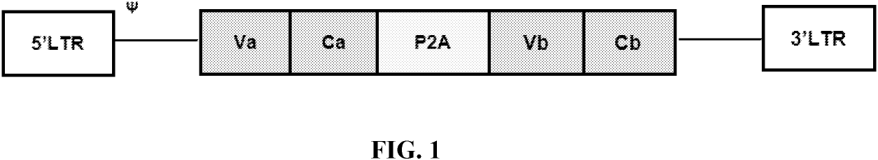

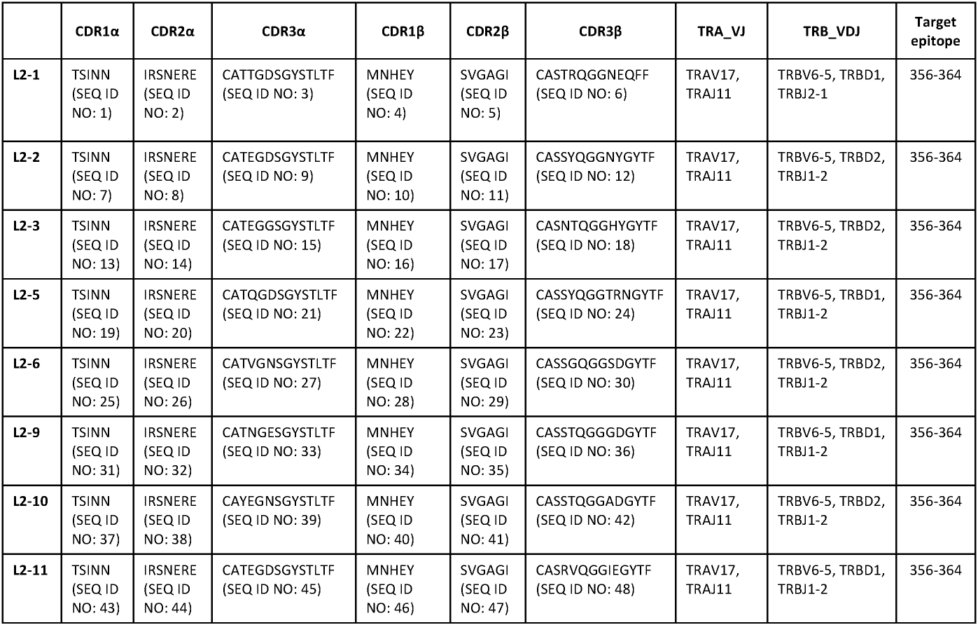

In some embodiments, a MP71 (or pMP71) vector is used. A MP71 retroviral vector construct is generated using standard molecular biology techniques. In some embodiments, the MP71 retroviral vector contains two genes linked by a P2A sequence: (1) the variable region of the alpha chain of a human anti-LMP2 TCR fused to the constant region of the mouse TCR alpha chain; (2) the variable region of the beta chain of same human anti-LMP2 TCR fused to the constant region of the mouse TCR beta chain. (FIG. 1)

In some embodiments, the vector can include an additional nucleic acid encoding an inhibitory protein (e.g., a checkpoint inhibitor). In various embodiments, the cell expresses the genetically engineered antigen receptor and the inhibitory protein. In various embodiments, the inhibitory protein is constitutively expressed.

In some embodiments, the vector or construct can contain a single promoter that drives the expression of one or more nucleic acid molecules. In some embodiments, such promoters can be multicistronic (bicistronic or tricistronic). For example, in some embodiments, transcription units can be engineered as a bicistronic unit containing an IRES (internal ribosome entry site), which allows coexpression of gene products (e.g. encoding an alpha chain and/or beta chain of a TCR) by a message from a single promoter. Alternatively, in some cases, a single promoter may direct expression of an RNA that contains, in a single open reading frame (ORF), two or three genes (e.g. encoding an alpha chain and/or beta chain of a TCR) separated from one another by sequences encoding a self-cleavage peptide (e.g., P2A or T2A) or a protease recognition site (e.g., furin). The ORF thus encodes a single polyprotein, which, either during (in the case of 2A e.g., T2A) or after translation, is cleaved into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream.

Various cell lines can be used in connection with the vectors as described herein. Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells; and NSO cells. In some embodiments, a particular eukaryotic host cell is selected based on its ability to make desired post-translational modifications to the binding molecule. For example, in some embodiments, CHO cells produce polypeptides that have a higher level of sialylation than the same polypeptide produced in 293 cells.

In one aspect, the disclosure also relates to a nucleic acid comprising a polynucleotide encoding a polypeptide comprising:
(1) a TCR a chain or a fragment thereof comprising an a chain variable region (Va) comprising complementarity determining regions (CDRs) 1, 2, and 3 comprising the amino acid sequences set forth in FIGS. 2-4, respectively, and wherein the Va, when paired with a corresponding b chain variable region (Vb), binds to LMP2; and/or
(2) a TCR b chain or a fragment thereof comprising a b chain variable region (Vb) comprising complementarity determining regions (CDRs) 1, 2, and 3 comprising the amino acid sequences set forth in FIGS. 2-4, respectively, and wherein the Vb, when paired with a corresponding a chain variable region (Va), binds to LMP2.

In some embodiments, the Va when paired with a Vb specifically binds to EBV LMP2, or the Vb when paired with a Va specifically binds to EBV LMP2. In some embodiments, the nucleic acid is cDNA.

In one aspect, the disclosure relates to a vector comprising one or more of the nucleic acids as described herein. In one aspect, the disclosure also relates to a vector comprising two of the nucleic acids as described herein. In some embodiments, the vector encodes the Va region and the Vb region that together bind to an EBV antigen.

In one aspect, the disclosure relates to a pair of vectors, wherein each vector comprises one of the nucleic acids as described herein, wherein together the pair of vectors encodes the Va region and the Vb region that together bind to an EBV antigen.

In one aspect, the disclosure relates to a cell comprising the vector or the pair of vectors as described herein. In some embodiments, the cell is a T cell.

In some cases, certain TCRs, may exhibit poor expression or activity in part due to mispairing and/or competition with endogenous TCR chains and/or other factors. One method to address these challenges has been to design recombinant TCRs with mouse constant domains to prevent mispairings with endogenous human TCR a or b chains. However, the use of recombinant TCRs with mouse sequences may present a risk for immune response. In some embodiments, a genetic disruption is introduced, e.g., by gene editing, at an endogenous gene encoding one or more TCR chains.

As shown in FIG. 1 the nucleic acid construct is cloned in a retroviral vector pMP71 containing two genes linked by a P2A sequence: (1) the variable region of the alpha chain of a human anti-LMP2 TCR fused to the constant region of the mouse TCR alpha chain; (2) the variable region of the beta chain of same human anti-LMP2 TCR fused to the constant region of the mouse TCR beta chain. In some embodiments, the nucleic acid construct further comprises a sequence encoding a signal peptide.

In some embodiments, the inhibitory protein is an anti-PD-1 antibody (e.g., an anti-PD-1 scFV).

The term “Linker” (L) or “linker domain” or “linker region” as used herein refer to an oligo- or polypeptide region from about 1 to 100 amino acids in length, which links together any of the domains/regions. Linkers can be composed of flexible residues like glycine and serine so that the adjacent protein domains are free to move relative to one another. Longer linkers can be used when it is desirable to ensure that two adjacent domains do not sterically interfere with one another. Linkers can be cleavable or non-cleavable. Examples of cleavable linkers include 2A linkers (for example P2A, T2A), 2A-like linkers or functional equivalents thereof and combinations thereof. In some embodiments, the linkers include the picornaviral 2A-like linker, CHYSEL sequences of porcine teschovirus (P2A), Thosea asigna virus (T2A) or combinations, variants and functional equivalents thereof. Other linkers will be apparent to those of skill in the art and can be used in the methods described herein.

The present disclosure also provides a nucleic acid sequence comprising a nucleotide sequence encoding any of the TCRs, antigen binding fragments thereof, and/or TCR-derivied binding molecules (including e.g., functional portions and functional variants thereof, polypeptides, or proteins described herein). “Nucleic acid” as used herein can include “polynucleotide,” “oligonucleotide,” and “nucleic acid molecule,” and generally means a polymer of DNA or RNA, which can be single-stranded or double-stranded, synthesized or obtained from natural sources, which can contain natural, non-natural or altered nucleotides. Furthermore, the nucleic acid comprises complementary DNA (cDNA). It is generally preferred that the nucleic acid does not comprise any insertions, deletions, inversions, and/or substitutions. However, it can be suitable in some instances, as discussed herein, for the nucleic acid to comprise one or more insertions, deletions, inversions, and/or substitutions.

The nucleic acids as described herein can be constructed based on chemical synthesis and/or enzymatic ligation reactions using procedures known in the art. For example, a nucleic acid can be chemically synthesized using naturally occurring nucleotides or variously modified nucleotides. In some of any such embodiments, the nucleotide sequence is codon-optimized.

The present disclosure also provides the nucleic acids comprising a nucleotide sequence complementary to the nucleotide sequence of any of the nucleic acids described herein or a nucleotide sequence which hybridizes under stringent conditions to the nucleotide sequence of any of the nucleic acids described herein.

In some embodiments, the nucleotide sequence encoding the alpha chain and the nucleotide sequence encoding the beta chain are separated by a peptide sequence that causes ribosome skipping. In some embodiments, the peptide that causes ribosome skipping is a P2A or T2A peptide. In some embodiments, the nucleic acid is synthetic. In some embodiments, the nucleic acid is cDNA.

In some embodiments, the vector can additionally include a nucleic acid sequence that encodes a checkpoint inhibitor (CPI) (e.g., an inhibitory protein). In some embodiments, the checkpoint inhibitor is e.g., any antibody or antigen binding fragment thereof as described herein. In some embodiments, the antibody or antigen binding fragments thereof can specifically bind to PD-1, PD-L1, PD-L2, 2B4 (CD244), 4-1BB, A2aR, B7.1, B7.2, B7-H2, B7-H3, B7-H4, B7-H6, BTLA, butyrophilins, CD160, CD48, CTLA4, GITR, gp49B, HHLA2, HVEM, ICOS, ILT-2, ILT-4, KIR family receptors, LAG-3, OX-40, PIR-B, SIRPalpha (CD47), TFM-4, TIGIT, TIM-1, TIM-3, TIM-4, or VISTA. In some embodiments, the inhibitory protein is a scFv (e.g., an anti-PD-1 scFv).

In some embodiments, the vector can additionally include a nucleic acid sequence that encodes a bifunctional trap fusion protein. In some embodiments, the bifunctional trap protein targets both the PD-1 and TGF-β. In some embodiments, the bifunctional trap protein targets both the PD-L1 and TGF-β. In some embodiments, the bifunctional fusion protein designed to block PD-L1 and sequester TGF-β. M7824 (MSB0011395C) comprises the extracellular domain of human TGF-β receptor II (TGFβRII) linked to the C-terminus of the human anti-PD-L1 scFv, based on the human IgG1 monoclonal antibody (mAb) avelumab. In some embodiments, the bifunctional fusion protein comprises the extracellular domain of human TGF-β receptor II (TGFβRII) linked to the C-terminus of the human anti-PD-1 scFv.

In some of any such embodiments, the TCR or antigen-binding fragment thereof is encoded by a nucleotide sequence that has been codon-optimized. In certain embodiments, the alpha and/or beta chain further comprises a signal peptide. In particular embodiments, the TCR or antigen-binding fragment thereof is isolated or purified or is recombinant. In some of any such embodiments, the TCR or antigen-binding fragment is recombinant. In some of any such embodiments, the TCR or antigen-binding fragment thereof is human.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein.

In some embodiments, the nucleic acid sequence is at least or about 10, 20, 30, 40, 50, 60, 70, 80, 90, 100, 110, 120, 130, 150, 200, 250, 300, 350, 400, 500, or 600 nucleotides. In some embodiments, the amino acid sequence is at least or about 5, 6, 7, 8, 9, 10, 20, 30, 40, 50, 60, 70, 80, 90, 100, 110, 120, 130, 140, 150, 160, 170, 180, 190, or 200 amino acid residues. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

### Methods of Generating T Cell Receptors and TCR-Like Molecules

The present disclosure also provides methods for identifying and generating T cell receptors that can recognize a target antigen. In some aspects, the methods involve subjecting biological samples containing T cells, such as primary T cells, including those derived from normal donors or patients having a disease or condition of interest, to multiple rounds of antigen exposure and assessment. In some aspects, the rounds involve the use of artificial or engineered antigen presenting cells, such as autologous dendritic cells or other APCs pulsed with a desired peptide antigen, to promote presentation on an MHC, such as a class I or II MHC.

In some aspects, multiple rounds of antigen exposure are carried out and in some aspects T cells are sorted following one or more of the rounds, e.g., based on ability to bind to the desired antigen (such as peptide-MHC tetramers).

Sorting can be carried out by methods known in the art, e.g., flow cytometry. Cells that can bind to the desired antigen (positive fraction) and cells that cannot effectively bind to the desired antigen (negative fraction) are analyzed, e.g., by single-cell sequencing methods. In some embodiments, sequencing is performed to identify, at a single-cell level, TCR pairs present in each sample. In some aspects, the methods can quantify the number of copies of a given TCR pair present in a sample, and as such can assess the abundance of a given TCR in a given sample, and/or enrichment thereof over another sample, such as enrichment or abundance in the positive (antigen-binding) fraction, e.g., over one or more rounds, for example, as compared to the negative fraction. Such assays can be performed to generate antigen-specific T cell receptors (TCRs). In some aspects, clonal T cell lines are generated and the sequences of individual paired TCR alpha and beta chains and abundance thereof in various populations are determined on a single-cell basis, using high-throughput paired TCR sequencing.

The TCR or antigen-binding fragment thereof can be further modified. In some embodiments, the binding molecules, e.g., TCRs or antigen-binding fragments thereof, include one or more amino acid variations, e.g., substitutions, deletions, insertions, and/or mutations, compared to the sequence of a binding molecule, e.g., any TCR described herein. Exemplary variants include those designed to improve the binding affinity and/or other biological properties of the binding molecule. Amino acid sequence variants of a binding molecule can be prepared by introducing appropriate modifications into the nucleotide sequence encoding the binding molecule, or by peptide synthesis. Such modifications include, for example, deletions from, and/or insertions into and/or substitutions of residues within the amino acid sequences of the binding molecule. Any combination of deletion, insertion, and substitution can be made to arrive at the final construct, provided that the final construct possesses the desired characteristics, e.g., specifically bind to the antigen.

Various binding molecules can be made from TCR. The binding molecules, e.g., TCRs or antigen-binding fragments thereof, can include one or more amino acid substitutions, e.g., as compared to a binding molecule, e.g., TCR, sequence described herein and/or compared to a sequence of a natural repertoire, e.g., human repertoire. Sites of interest for substitutional mutagenesis include the CDRs, FRs and /or constant regions. Amino acid substitutions can be introduced into a binding molecule of interest and the products screened for a desired activity, e.g., retained/improved antigen affinity or avidity, decreased immunogenicity, improved half-life, CD8-independent binding or activity, surface expression, promotion of TCR chain pairing and/or other improved properties or functions.

In some embodiments, one or more residues within a CDR of a parent binding molecule, e.g., TCR, is/are substituted. In some embodiments, the substitution is made to revert a sequence or position in the sequence to a germline sequence, such as a binding molecule sequence found in the germline (e.g., human germline), for example, to reduce the likelihood of immunogenicity, e.g., upon administration to a human subject.

In some embodiments, a functional variant is made from a TCR or a TCR-derived binding molecule. The term “functional variant,” as used herein, refers to a binding molecule having an adequate or significant sequence identity to a parent molecule. Further, the functional variant retains the same biological activity as of the parent protein. The functional variant encompasses those variants of the TCR protein described herein (the parent TCR, polypeptide, or protein) that retain the ability to specifically bind to EBV epitope for which the parent TCR has antigenic specificity or to which the parent polypeptide or protein specifically binds. Furthermore the binding region (e.g., variable domain) of the functional variant can be to a similar extent, the same extent, or to a higher extent, as the parent TCR protein. In reference to the parent TCR, polypeptide, or protein, the functional variant can, for instance, be at least about 30%, 50%, 75%, 80%, 90%, 95%, 96%, 97%, 98%, 99% or more identical in amino acid sequence to the parent TCR, polypeptide, or protein.

Substitutions, insertions, or deletions can be made to one or more CDRs so long as such alterations do not substantially reduce the ability of the binding molecule, e.g., TCR or antigen-binding fragment thereof, to bind antigen. For example, conservative alterations (e.g., conservative substitutions as provided herein) that do not substantially reduce binding affinity can be made in CDRs. Such alterations can, for example, be outside of antigen contacting residues in the CDRs. In certain embodiments of the variable sequences provided herein, each CDR either is unaltered, or contains no more than one, two or three amino acid substitutions.

### TCR-Derived Antibodies

The present disclosure also provides an antibody or antigen-binding fragment thereof that contains any one or more of the CDRs as described above. In some embodiments, the antibody or antigen-binding fragment contains variable heavy and light chain containing a CDR1, a CDR2 and/or a CDR3 contained in the alpha chain and a CDR1, a CDR2 and/or a CDR3 contained in the beta chain. In some embodiments, the antibody or antigen-binding fragment contains one or more CDRs that are at least at or about 80%, 85%, 90, 91, 92, 93, 94, 95, 96, 97, 98, or 99% identical to CDR sequences in FIGS. 2-4.

In some embodiments, the antibodies and antigen binding fragments thereof, e.g. TCR-like antibodies, specifically recognize a peptide epitope (e.g., EBV antigen) in the context of an MHC molecule, such as an MHC class I. In some cases, the MHC class I molecule is an HLA-A2 molecule, e.g. HLA-A2*01.

In some embodiments, the antibodies and antigen binding fragments thereof can specifically recognize a peptide epitope (e.g., EBV antigen) in an MHC molecule independent manner.

In general, antibodies (also called immunoglobulins) are made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani,et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody’s target molecule. It includes, e.g., Fab, Fab′, F(ab′)2, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

In some embodiments, the scFV comprises one heavy chain variable domain, and one light chain variable domain. In some embodiments, the scFV comprises two heavy chain variable domains, and two light chain variable domains.

### TCR-Derived CAR

The antibody or antigen-binding portion thereof can be expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against a peptide in the context of an MHC molecule can also be referred to as a TCR-like CAR. Thus, among the provided binding molecules, e.g., EBV binding molecules, are antigen receptors, such as those that include one of the provided antibodies, e.g., TCR-like antibodies. In some embodiments, the antigen receptors and other chimeric receptors specifically bind to a region or epitope of LMP2, e.g. TCR-like antibodies. Among the antigen receptors are functional non-TCR antigen receptors, such as chimeric antigen receptors (CARs). Also provided are cells expressing the CARs and uses thereof in adoptive cell therapy, such as treatment of diseases and disorders associated with EBV antigen expression.

TCR-like CARs that contain a non-TCR molecule that exhibits T cell receptor specificity, such as for a T cell epitope or peptide epitope when displayed or presented in the context of an MHC molecule. In some embodiments, a TCR-like CAR can contain an antibody or antigen-binding portion thereof, e.g., TCR-like antibody, such as described herein. In some embodiments, the antibody or antibody-binding portion thereof is reactive against specific peptide epitope in the context of an MHC molecule, wherein the antibody or antibody fragment can differentiate the specific peptide in the context of the MHC molecule from the MHC molecule alone, the specific peptide alone, and, in some cases, an irrelevant peptide in the context of an MHC molecule. In some embodiments, an antibody or antigen-binding portion thereof can exhibit a higher binding affinity than a T cell receptor.

Exemplary antigen receptors, including CARs, and methods for engineering and introducing such receptors into cells, include those described, for example, in US2002/131960, US2013/287748, US2013/0149337, U.S. 6,451,995, U.S. 7,446,190, U.S. 8,252,592; each of which is incorporated herein by reference in its entirety.

In some embodiments, the CARs generally include an extracellular antigen (or ligand) binding domain, including e.g., an antibody or antigen-binding fragment thereof specific for a peptide, linked to one or more intracellular signaling components, in some aspects via linkers and/or transmembrane domain(s). In some embodiments, such molecules can typically mimic or approximate a signal through a natural antigen receptor, such as a TCR, and, optionally, a signal through such a receptor in combination with a co-stimulatory receptor.

In some embodiments, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAh). In some embodiments, the CAR contains a TCR-like antibody, such as an antibody or an antigen-binding fragment (e.g., scFv) that specifically recognizes a peptide epitope presented on the cell surface in the context of an MHC molecule.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD 137 (4-lBB, TNFRSF9) co-stimulatory domains, linked to a CD3 zeta intracellular domain.

In some embodiments, the binding molecule can also be a genetically engineered T cell receptor (TCR), genetically engineered NK cell receptor, killer-cell immunoglobulin-like receptor (KIR), C-type lectin receptor, leukocyte immunoglobulin-like receptor (LILR), Type 1 cytokine receptor, Type 2 cytokine receptor, tumor necrosis factor family, TGFβ receptor, chemokine receptor, or a member of immunoglobulins superfamily (IgSF).

In some embodiments, the engineered cells are further modified in any number of ways, such that their therapeutic or prophylactic efficacy is increased. For example, the engineered TCR or other binding molecules expressed by the population can be conjugated either directly or indirectly through a linker to a targeting moiety. The practice of conjugating binding molecules, e.g., the CAR or TCR, to targeting moieties is known in the art, and are described e.g., in Wadhwa et al. “Receptor mediated glycotargeting.” Journal of drug targeting 3.2 (1995): 111-127., and U.S. Pat. No. 5,087,616; which are incorporated herein by reference in the entirety.

### Method for Preparation of Engineered Cells

The present disclosure provides a method or process for manufacturing and using the engineered cells for treatment of pathological diseases or conditions.

The cells for introduction of the binding molecule, e.g., TCR, can be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, or non-human primate.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge. In some aspects, a washing step is accomplished by tangential flow filtration (TFF). In some embodiments, the cells are resuspended in a variety of biocompatible buffers after washing, such as, for example, Ca ²⁺/Mg ²⁺ free PBS. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In some embodiments, the method comprises one or more steps of: e.g., isolating the T cells from a patient’s blood; transducing the population T cells with a viral vector including the nucleic acid construct encoding a genetically engineered antigen receptor; expanding the transduced cells in vitro; and/or infusing the expanded cells into the patient, where the engineered T cells will seek and destroy antigen positive tumor cells. In some embodiments, the nucleic acid construct further includes a sequence encoding an inhibitory protein. In some embodiments, these engineered T cells can block PD-⅟PD-L1 immunosuppression and strengthen the antitumor immune response. In some embodiments, the method further comprises: transfection of T cells with the viral vector containing the nucleic acid construct.

In some embodiments, the methods involve introducing any vectors described herein into a cell in vitro or ex vivo. In some embodiments, the vector is a viral vector and the introducing is carried out by transduction. In some embodiments, the methods further involve introducing into the cell one or more agent, wherein each of the one or more agent is independently capable of inducing a genetic disruption of a T cell receptor alpha constant (TRAC) gene and/or a T cell receptor beta constant (TRBC) gene. In some embodiments, the one or more agent is an inhibitory nucleic acid (e.g., siRNA). In some embodiments, the one or more agent is a fusion protein comprising a DNA-targeting protein and a nuclease or an RNA-guided nuclease (e.g., a clustered regularly interspaced short palindromic nucleic acid (CRISPR)-associated nuclease).

The transfection of T cells may be achieved by using any standard method such as calcium phosphate, electroporation, liposomal mediated transfer, microinjection, biolistic particle delivery system, or any other known methods by skilled artisan. In some embodiments, transfection of T cells is performed using the calcium phosphate method.

According to various embodiments described herein, the present disclosure provides an immunotherapy against tumors, particularly EBV associated cancers. In some embodiments, the engineered T cells recognize a tumor associated EBV antigen and simultaneously secrete a single-chain antibody (scFv) fusion protein that blocks Programmed Cell Death Protein 1 (PD-1) and TGFβ. These engineered T cells demonstrate a stronger antitumor response and reduced T cell exhaustion. It has been found experimentally that PD-1 checkpoint blockade is more effective in the methods described herein because anti-PD-1 agent delivery is localized to the tumor site, thus has a higher concentration at the tumor site. Also, toxicity due to non-specific inflammation is reduced because anti-PD-1 drug delivery is localized to the tumor site. The present disclosure provides that combination of anti-EBV TCR and anti- PD-1 antibody improves T cell activation and/or prevents T cell exhaustion compared to existing alternatives.

The present disclosure provides a method to create a personalized anti-tumor immunotherapy. Genetically engineered anti-LMP2 T cells can be produced from a patient’s blood cells. These engineered T cells are then reinfused into the patient as a cellular therapy product. This product can be applied to any patient who has an EBV associated tumor, including, but are not limited to nasopharyngeal carcinoma, Hodgkin’s lymphoma, Burkitt’s lymphoma, and stomach cancer.

Methods of preparing engineered cells and administering these engineered cells to a subject are known in the art, and are described e.g., in US Pat. No. 10,174,098 and Draper et al. “Targeting Of HPV-16+ Epithelial Cancer Cells By Tcr Gene Engineered t Cells Directed Against e6.” Clinical Cancer Research 21.19 (2015): 4431-4439, both of which are incorporated by reference in their entirety.

### Methods of Treatment

The methods disclosed herein can be used for various therapeutic purposes. In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of engineered cells expressing TCR, antigen binding fragments thereof, and TCR-derived binding molecules to a subject in need thereof (e.g., a subject having, or identified or diagnosed as having, a cancer), e.g., an EBV-associated cancer. In some embodiments, the EBV-associated cancer is nasopharyngeal carcinoma, Hodgkin’s lymphoma, Burkitt’s lymphoma, and stomach cancer.

In some embodiments, the subject has a solid tumor. In some embodiments, the subject has breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy. In some embodiments, the cancer is unresectable melanoma or metastatic melanoma, non-small cell lung carcinoma (NSCLC), small cell lung cancer (SCLC), bladder cancer, or metastatic hormone-refractory prostate cancer.

In some embodiments, the compositions and methods disclosed herein can be used for treatment of patients at risk for a cancer. Patients with cancer can be identified with various methods known in the art.

Furthermore, the disclosure provides methods for treating infection or infection associated conditions in a subject. In some embodiments, the treatment can halt, slow, retard, or inhibit progression of the disease. These methods generally involve administering a therapeutically effective amount of genetic engineered cells disclosed herein to a subject in need thereof. In some embodiments, the disease or condition treated is an infectious disease or condition, such as, but not limited to, viral, retroviral, bacterial, and protozoal infections, immunodeficiency, Human Papilloma Virus (HPV), Cytomegalovirus (CMV), Epstein-Barr virus (EBV), adenovirus, BK polyomavirus. In some embodiments, the disease is EBV infection.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the therapeutic agent and/or therapeutic compositions is to be administered, a severity of symptoms and a route of administration, and thus administration can be determined on an individual basis.

An effective amount can be administered in one or more administrations. By way of example, an effective amount of a composition is an amount sufficient to ameliorate, stop, stabilize, reverse, inhibit, slow and/or delay progression of a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective may vary, depending on, inter alia, patient history as well as other factors such as the type (and/or dosage) of compositions used.

Effective amounts and schedules for administrations may be determined empirically, and making such determinations is within the skill in the art. Those skilled in the art will understand that the dosage that must be administered will vary depending on, for example, the mammal that will receive the treatment, the route of administration, the particular type of therapeutic agents and other drugs being administered to the mammal. Guidance in selecting appropriate doses can be found in the literature. In addition, a treatment does not necessarily result in the 100% or complete treatment or prevention of a disease or a condition. There are multiple treatment/prevention methods available with a varying degree of therapeutic effect which one of ordinary skill in the art recognizes as a potentially advantageous therapeutic mean.

In some aspects, the present disclosure also provides methods of diagnosing a disease/condition in a mammal, wherein the TCRs, antigen binding fragments, TCR-derived binding molecules interact with the sample(s) obtained from a subject to form a complex, wherein the sample can comprise one more cells, polypeptides, proteins, nucleic acids, antibodies, or antigen binding portions, blood, whole cells, lysates thereof, or a fraction of the whole cell lysates, e.g., a nuclear or cytoplasmic fraction, a whole protein fraction, or a nucleic acid fraction thereof, wherein the detection of the complex is the indicative of presence of a condition in the mammal, wherein the condition is cancer, EBV infection, or EBV-positive premalignancy. Further, the detection of the complex can be in any number of way known in the art but not limited to, ELISA, Flow cytometery, Fluorescence in situ hybridization (FISH), Polymerase chain reaction (PCR), microarray, southern blotting, electrophoresis, Phage analysis, chromatography and more. Thus, the treatment methods can further include determining whether a subject can benefit from a treatment as disclosed herein, e.g., by determining whether the subject has EBV infection or EBV-associated cancer.

In any of the methods described herein, the engineered cells and, and/or at least one additional therapeutic agent can be administered to the subject at least once a week (e.g., once a week, twice a week, three times a week, four times a week, once a day, twice a day, or three times a day). In some embodiments, at least two different engineered cells (e.g., cells express different binding molecules) are administered in the same composition (e.g., a liquid composition). In some embodiments, engineered cells and at least one additional therapeutic agent are administered in the same composition (e.g., a liquid composition). In some embodiments, engineered cells and the at least one additional therapeutic agent are administered in two different compositions. In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, concurrently with, or after administering the engineered cells to the subject.

In some embodiments, one or more additional therapeutic agents can be administered to the subject. The additional therapeutic agent can be a checkpoint inhibitor (CPI). In some embodiments, the checkpoint inhibitor is an inhibitory protein, e.g., an antibody or antigen binding fragment thereof. The checkpoint inhibitor can inhibit or block one or more immune checkpoints, including e.g., PD-1, PD-L1, PD-L2, 2B4 (CD244), 4-1BB, A2aR, B7.1, B7.2, B7-H2, B7-H3, B7-H4, B7-H6, BTLA, butyrophilins, CD160, CD48, CTLA4, GITR, gp49B, HHLA2, HVEM, ICOS, ILT-2, ILT-4, KIR family receptors, LAG-3, OX-40, PIR-B, SIRPalpha (CD47), TFM-4, TIGIT, TIM-1, TIM-3, TIM-4, VISTA and combinations thereof. In some embodiments, the inhibitory protein blocks PD-1 or PD-Ll. In various embodiments, the inhibitory protein comprises an anti-PD-1 scFv. The inhibitory protein is capable of leading to reduced expression of PD-1 or PD-L1 and/or inhibiting upregulation of PD- 1 or PD-L1 in T cells in the population and/or physically obstructing the formation of the PD- ⅟PD-L1 complex and subsequent signal transduction. In some embodiments, the inhibitory protein blocks PD-1. In some embodiments, the additional therapeutic agent is an anti-OX40 antibody, an anti-PD-L1 antibody, an anti-PD-L2 antibody, an anti-LAG-3 antibody, an anti-TIGIT antibody, an anti-BTLA antibody, an anti-CTLA-4 antibody, or an anti-GITR antibody. In some embodiments, the additional therapeutic agent is an anti-CTLA4 antibody (e.g., ipilimumab), an anti-CD20 antibody (e.g., rituximab), an anti- EGFR antibody (e.g., cetuximab), an anti-CD319 antibody (e.g., elotuzumab), or an anti-PD1 antibody (e.g., nivolumab).

In some embodiments, the additional therapeutic agent is a bifunctional trap fusion protein. Bifunctional trap proteins can target both immune checkpoints and TGF-β negative regulatory pathways. In addition to expression of immune checkpoints, the tumor microenvironment contains other immunosuppressive molecules. Of particular interest is the cytokine TGF-β (TGFB), which has multiple functions in cancer. TGF-β prevents proliferation and promotes differentiation and apoptosis of tumor cells early in tumor development. However, during tumor progression, tumor TGF-β insensitivity arises due to the loss of TGF-β receptor expression or mutation to downstream signaling elements. TGF-β then promotes tumor progression through its effects on angiogenesis, induction of epithelial-to-mesenchymal transition (EMT), and immune suppression. High TGF-β serum level and loss of TGF-β receptor (TGFβR) expression on tumors correlates with poor prognosis. TGFβ-targeted therapies have demonstrated limited clinical activity. In some embodiments, the bifunctional trap protein targets both the PD-1 and TGF-β. In some embodiments, the bifunctional trap protein targets both the PD-L1 and TGF-β. In some embodiments, the bifunctional fusion protein designed to block PD-L1 and sequester TGF-β. M7824 (MSB0011395C) comprises the extracellular domain of human TGF-β receptor II (TGFβRII) linked to the C-terminus of the human anti-PD-L1 scFv, based on the human IgG1 monoclonal antibody (mAb) avelumab. In some embodiments, the bifunctional fusion protein comprises the extracellular domain of human TGF-β receptor II (TGFβRII) linked to the C-terminus of the human anti-PD-1 scFv. These bifunctional trap fusion proteins are described e.g., Knudson, et al. “M7824, a novel bifunctional anti-PD-L1/TGFβ Trap fusion protein, promotes anti-tumor efficacy as monotherapy and in combination with vaccine.” Oncoimmunology 7.5 (2018): e1426519, which is incorporated herein by reference in its entirety. In some embodiments, the subject is treated by cells that express TCR or antigen-binding molecules as described herein and one or more bifunctional trap fusion proteins.

In one some embodiments, the additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton’s tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat). In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject. In some embodiments, the additional therapeutic agent is selected from asparaginase, busulfan, carboplatin, cisplatin, daunorubicin, doxorubicin, fluorouracil, gemcitabine, hydroxyurea, methotrexate, paclitaxel, rituximab, vinblastine, vincristine and/or combinations thereof.

### Compositions and Formulations

The present disclosure provides compositions (including pharmaceutical and therapeutic compositions) containing the engineered cells and populations thereof, produced by the methods disclosed herein. Also provided are methods, e.g., therapeutic methods for administrating the engineered T cells and compositions thereof to subjects, e.g., patients.

Compositions including the engineered T cells for administration, including pharmaceutical compositions and formulations, such as unit dose form compositions including the number of cells for administration in a given dose or fraction thereof are provided. The pharmaceutical compositions and formulations can include one or more optional pharmaceutically acceptable carrier or excipient. In some embodiments, the composition includes at least one additional therapeutic agent.

A pharmaceutically acceptable carrier refers to an ingredient in a pharmaceutical composition, other than an active ingredient. The pharmaceutically acceptable carrier does not interfere with the active ingredient and is nontoxic to a subject. A pharmaceutically acceptable carrier can include, but is not limited to, a buffer, excipient, stabilizer, or preservative. The pharmaceutical formulation refers to process in which different substances and/or agents are combined to produce a final medicinal product. The formulation studies involve developing a preparation of drug acceptable for patient. Additionally, a preparation which is in such form as to permit the biological activity of an active ingredient contained therein to be effective, and which contains no additional components which are unacceptably toxic to a subject to which the formulation would be administered.

In some embodiments, the choice of carrier is determined in part by the particular cell (e.g., T cell or NK cell) and/or by the method of administration. A variety of suitable formulations are available. For example, the pharmaceutical composition can contain preservatives. Suitable preservatives can include, for example, methylparaben, propylparaben, sodium benzoate, and benzalkonium chloride. In some embodiments, a mixture of two or more preservatives is used. The preservative or mixtures thereof are typically present in an amount of about 0.0001% to about 2% by weight of the total composition. Carriers are described, e.g., by Remington’s Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Suitable buffering agents include, for example, citric acid, sodium citrate, phosphoric acid, potassium phosphate, and various other acids and salts. In some embodiments, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition can also contain more than one active ingredient useful for a particular indication, disease, or condition being treated with the engineered cells, preferably those with activities complementary to the cells, where the respective activities do not adversely affect one another. Such active ingredients are suitably present in combination in amounts that are effective for the purpose intended. Thus, in some embodiments, the pharmaceutical composition can further include other pharmaceutically active agents or drugs, such as checkpoint inhibitors, fusion proteins, chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

The cells and compositions can be administered using standard administration techniques, formulations, and/or devices. Administration of the cells can be autologous or heterologous. For example, immunoresponsive T cells or progenitors can be obtained from one subject, and administered to the same subject or a different, compatible subject after genetically modifying them in accordance with various embodiments described herein. Peripheral blood derived immunoresponsive T cells or their progeny (e.g., in vivo, ex vivo or in vitro derived) can be administered via localized injection, including catheter administration, systemic injection, localized injection, intravenous injection, or parenteral administration. Usually, when administering a therapeutic composition (e.g., a pharmaceutical composition containing a genetically modified immunoresponsive cell), it is generally formulated in a unit dosage injectable form (solution, suspension, emulsion).

Formulations disclosed herein include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection.

The compositions in some embodiments are provided as sterile liquid preparations, e.g., isotonic aqueous solutions, suspensions, emulsions, dispersions, or viscous compositions, which can in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts can in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility can be readily accomplished, e.g., by filtration through sterile filtration membranes.

The compositions or pharmaceutical compositions as described herein can be included in a container, pack, or dispenser together with instructions for administration.

### Methods of Administration

Provided are also methods of administering the cells, populations, and compositions, and uses of such cells, populations, and compositions to treat or prevent diseases, conditions, and disorders, including cancers. In some embodiments, the methods described herein can reduce the risk of the developing diseases, conditions, and disorders as described herein.

In some embodiments, the cells, populations, and compositions, described herein are administered to a subject or patient having a particular disease or condition to be treated, e.g., via adoptive cell therapy, such as adoptive T cell therapy. In some embodiments, cells and compositions prepared by the provided methods, such as engineered compositions and end-of-production compositions following incubation and/or other processing steps, are administered to a subject, such as a subject having or at risk for the disease or condition. In some aspects, the methods thereby treat, e.g., ameliorate one or more symptom of, the disease or condition, such as by lessening tumor burden in cancer expressing an antigen recognized by the engineered T cells.

Methods for administration of cells for adoptive cell therapy are known and can be used in connection with the provided methods and compositions. For example, adoptive T cell therapy methods are described, e.g., in U.S. 2003/0170238; U.S. Pat. No. 4,690,915; Rosenberg, “Cell transfer immunotherapy for metastatic solid cancer—what clinicians need to know.” Nature reviews Clinical oncology 8.10 (2011): 577; Themeli et al. “Generation of tumor-targeted human T lymphocytes from induced pluripotent stem cells for cancer therapy.” Nature biotechnology 31.10 (2013): 928; Tsukahara et al. “CD19 target-engineered T-cells accumulate at tumor lesions in human B-cell lymphoma xenograft mouse models.” Biochemical and biophysical research communications 438.1 (2013): 84-89; Davila et al. “CD19 CAR-targeted T cells induce long-term remission and B Cell Aplasia in an immunocompetent mouse model of B cell acute lymphoblastic leukemia.” PloS one 8.4 (2013); each of which is incorporated herein by reference in its entirety.

In some embodiments, the cell therapy, e.g., adoptive T cell therapy, is carried out by autologous transfer, in which the T cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the T cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the HLA class or HLA supertype of the subject is identified. In some embodiments, the subject is treated with a cell therapy that can recognize the antigen in the context of the HLA class or HLA supertype.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at high risk of relapse, and thus the cells are administered prophylactically, e.g., to reduce the likelihood of or prevent relapse. In some embodiments, the subject has not received prior treatment with another therapeutic agent.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8+ and CD4+ T cells, are administered at or within a tolerated difference of a desired dose of total cells, such as a desired dose of T cells. In some embodiments, the desired dose is a desired number of cells or a desired number of cells per unit of body weight of the subject to whom the cells are administered, e.g., cells/kg. In some embodiments, the desired dose is at or above a minimum number of cells or minimum number of cells per unit of body weight. In some embodiments, among the total cells, administered at the desired dose, the individual populations or sub- types are present at or near a desired output ratio (such as CD4+ to CD8+ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some embodiments, the desired dose is a desired number of cells of the sub-type or population, or a desired number of such cells per unit of body weight of the subject to whom the cells are administered, e.g., cells/kg. In some embodiments, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4+ to CD8+ cells, and/or is based on a desired fixed or minimum dose of CD4+ and/or CD8+ cells.

In certain embodiments, the cells or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub- populations of cells is within a range of between at or about 10⁴ and at or about 10⁹ cells/kilograms (kg) body weight, such as between 10⁵ and 10⁶ cells/kg body weight, for example, at least or at least about or at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at least or at least about or at or about 1×10⁵ T cells/kg, 1.5×10⁵ T cells/kg, 2×10⁵ T cells/kg, or 1×10⁶ T cells/kg body weight.

In some embodiments, the cells are administered at or within a certain range of error of between at or about 10⁴ and at or about 10⁹ CD4+ and/or CD8+ cells/kilograms (kg) body weight, such as between 10⁵ and 10⁶ CD4+ and/or CD8+ cells/kg body weight, for example, at least or at least about or at or about 1×10⁵ CD4+ and/or CD8+ cells/kg, 1.5×10⁵ CD4+ and/or CD8+ cells/kg, 2×10⁵ CD4+ and/or CD8+ cells/kg, or 1×10⁶ CD4+ and/or CD8+ cells/kg body weight.

In some embodiments, the cells are administered at or within a certain range of error of, greater than, and/or at least about 1×10⁶, about 2.5×10⁶, about 5×10⁶, about 7.5×10⁶, or about 9×10⁶ CD4+ cells, and/or at least about 1×10⁶, about 2.5×10⁶, about 5×10⁶, about 7.5×10⁶, or about 9×10⁶ CD8+ cells, and/or at least about 1×10⁶, about 2.5×10⁶, about 5×10⁶, about 7.5×10⁶, or about 9×10⁶ T cells. In some embodiments, the cells are administered at or within a certain range of error of between about 10⁸ and 10¹² or between about 10¹⁰ and 10¹¹ T cells, between about 10⁸ and 10¹² or between about 10¹⁰ and 10¹¹ CD4+ cells, and/or between about 10⁸ and 10¹² or between about 10¹⁰ and 10¹¹ CD8+ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4+ to CD8+ cells) is between at or about 1:5 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9: 1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges. In some aspects, the TCR described here provides improved expression and activity, thereby providing therapeutic effects even at a low effector to target (E:T) ratio.

Optimal response to therapy can depend on the ability of the engineered recombinant receptors such as TCRs, to be consistently and reliably expressed on the surface of the cells and/or bind the target antigen. For example, in some cases, properties of certain recombinant receptors, e.g., TCRs, can affect the expression and/or activity of the recombinant receptor, in some cases when expressed in a cell, such as a human T cell, used in cell therapy. In some contexts, the level of expression of particular recombinant receptors, e.g., TCRs, can be low, and activity of the engineered cells, such as human T cells, expressing such recombinant receptors, may be limited due to poor expression or poor signaling activity. In some cases, consistency and/or efficiency of expression of the recombinant receptor, and activity of the receptor is limited in certain cells or certain cell populations of available therapeutic approaches. In some cases, a large number of engineered T cells (a high effector to target (E:T) ratio) is required to exhibit functional activity. In some embodiments, the desired ratio (E:T ratio) is between at or about 1:10 and at or about 10:1 (or greater than about 1:10 and less than about 10:1), or between at or about 1:1 and at or about 10:1 (or greater than about 1:1 and less than about 5:1), such as between at or about 2:1 and at or about 10:1. In some embodiments, the E:T ratio is greater than or about 1:1, 2:1, 3:1, 4:1, 5:1, 6:1, 7:1, 8:1, 9:1, or 10:1.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject’s clinical history and response to the cells, and the discretion of the attending physician. The compositions and cells are in some embodiments suitably administered to the subject at one time or over a series of treatments.

The cells described herein can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon’s injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co- administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

Following administration of the cells, the biological activity of the engineered cell populations in some embodiments is measured, e.g., by any of a number of known methods. Parameters to assess include specific binding of engineered T cells to the antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al. “Construction and pre-clinical evaluation of an anti-CD19 chimeric antigen receptor.” Journal of immunotherapy (Hagerstown, Md.: 1997) 32.7 (2009): 689 and Hermans et al. “The VITAL assay: a versatile fluorometric technique for assessing CTL-and NKT-mediated cytotoxicity against multiple targets in vitro and in vivo.” Journal of immunological methods 285.1 (2004): 25-40. In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

### Dosing Schedule and Treatment Regimens

Repeated dosing methods are provided in which a first dose of cells is given followed by one or more second consecutive doses. The timing and size of the multiple doses of cells generally are designed to increase the efficacy and/or activity and/or function of engineered cells as described herein, when administered to a subject in adoptive therapy methods. In some embodiments, the repeated dosing reduce the downregulation or inhibiting activity that can occur when inhibitory immune molecules, such as PD-1 and/or PD-L1 are upregulated on engineered T cells. The methods involve administering a first dose, generally followed by one or more consecutive doses, with particular time frames between the different doses.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time (e.g., no more than 3 days). Thus, in some contexts, the first or consecutive dose is a single or continuous administration of the specified number of cells, given or initiated at a single point in time. In some contexts, however, the first or consecutive dose is administered in multiple injections or infusions over a limited time period (e.g., no more than three days), such as once a day for three days or for two days or by multiple infusions over a single day period.

The cells of the first dose are administered in a single pharmaceutical composition. In some embodiments, the cells of the consecutive dose are administered in a single pharmaceutical composition.

In some embodiments, the cells of the first dose are administered in a plurality of compositions, collectively containing the cells of the first dose. In some embodiments, the cells of the consecutive dose are administered in a plurality of compositions, collectively containing the cells of the consecutive dose. In some aspects, additional consecutive doses can be administered in a plurality of compositions over a period of no more than 3 days.

The term “split dose” refers to a dose that is split so that it is administered over more than one day. This type of dosing is encompassed by the present methods and is considered to be a single dose. Thus, in some embodiments, the first dose and/or consecutive dose(s) can be administered as a split dose. For example, in some embodiments, the dose can be administered to the subject over 2 days or over 3 days. Exemplary methods for split dosing include administering 25% of the dose on the first day and administering the remaining 75% of the dose on the second day. In other embodiments, 33% of the first dose can be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

With reference to a prior dose, such as a first dose, the term “consecutive dose” refers to a dose that is administered to the same subject after the prior, e.g., first, dose without any intervening doses having been administered to the subject in the interim. Nonetheless, the term does not encompass the second, third, and/or so forth, injection or infusion in a series of infusions or injections comprised within a single split dose. Thus, unless otherwise specified, a second infusion within a one, two or three-day period is not considered to be a “consecutive” dose as used herein. Likewise, a second, third, and so-forth in the series of multiple doses within a split dose also is not considered to be an “intervening” dose in the context of the meaning of “consecutive” dose. Thus, unless otherwise specified, a dose administered a certain period of time, greater than three days, after the initiation of a first or prior dose, is considered to be a “consecutive” dose even if the subject receives a second or subsequent injection or infusion of the cells following the initiation of the first dose, so long as the second or subsequent injection or infusion occurred within the three-day period following the initiation of the first or prior dose.

Thus, unless otherwise specified, multiple administrations of the same cells over a period of up to 3 days is considered to be a single dose, and administration of cells within 3 days of an initial administration is not considered a consecutive dose and is not considered to be an intervening dose for purposes of determining whether a second dose is “consecutive” to the first.

In some embodiments, multiple consecutive doses are given, in some aspects using the same timing guidelines as those with respect to the timing between the first dose and first consecutive dose, e.g., by administering a first and multiple consecutive doses, with each consecutive dose given within a period of time in which an inhibitory immune molecule, such as PD-1 and/or PD-L1, has been upregulated in cells in the subject from an administered first dose. It is within the level of a skilled artisan to empirically determine when to provide a consecutive dose, such as by assessing levels of PD-1 and/or PD-L1 in antigen-expressing, such as TCR-expressing cells, from peripheral blood or other bodily fluid.

In some embodiments, the timing between the first dose and first consecutive dose, or a first and multiple consecutive doses, is such that each consecutive dose is given within a period of time is greater than about 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days or more. In some embodiments, the consecutive dose is given within a time period that is less than about 28 days after the administration of the first or immediately prior dose. The additional multiple additional consecutive dose or doses also are referred to as subsequent dose or subsequent consecutive dose.

The size of the first and/or one or more consecutive doses of cells are generally designed to provide improved efficacy and/or reduced risk of toxicity. In some aspects, a dosage amount or size of a first dose or any consecutive dose is any dosage or amount as described above. In some embodiments, the number of cells in the first dose or in any consecutive dose is between about 0.5×10⁶ cells/kg body weight of the subject and 5×10⁶ cells/kg, between about 0.75×10⁶ cells/kg and 3×10⁶ cells/kg or between about 1×10⁶ cells/kg and 2×10⁶ cells/kg.

As used herein, “first dose” is used to describe the timing of a given dose being prior to the administration of a consecutive or subsequent dose. The term does not necessarily imply that the subject has never before received a dose of cell therapy or even that the subject has not before received a dose of the same cells or cells expressing the same recombinant receptor or targeting the same antigen.

In some embodiments, multiple doses can be administered to a subject over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer).

In some embodiments, the engineered receptor, e.g., the TCR, expressed by the cells in the consecutive dose contains at least one immunoreactive epitope as the receptor, e.g., the TCR, expressed by the cells of the first dose. In some embodiments, the receptor, e.g., the TCR, expressed by the cells administered in the consecutive dose is identical to the receptor, e.g., the TCR, expressed by the first dose or is substantially identical to the receptor, e.g., the TCR, expressed by the cells of administered in the first dose.

The receptors, such as TCRs, expressed by the cells administered to the subject in the various doses generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells in the first dose express a TCRs that specifically binds to an antigen expressed.

### EXAMPLES

The invention is further described in the following examples, which do not limit the scope of the invention described in the claims.

### EXAMPLE 1. Materials and Methods

### Construct Design

For LMP2 TCR-T cells, a pMP71 retroviral vector construct containing 2 coding regions was generated using standard molecular biology techniques. As shown in FIG. 1, the retroviral vector construct contains two coding regions linked by a P2A sequence: (1) the variable region of the alpha chain of a human anti-LMP2 TCR fused to the constant region of the mouse TCR alpha chain; (2) the variable region of the beta chain of same human anti-LMP2 TCR fused to the constant region of the mouse TCR beta chain. The construct was cloned into a pMP71 retroviral vector to produce TCR-T cells.

### Cell Lines and Media

HEK-293T, HMy2.CIR, and K562 cells were obtained from ATCC. Peripheral blood mononuclear cells (PBMCs) from anonymous donors were obtained from Hemacare. HMy2.CIR-A2 cells were produced by retroviral transduction of HMy2.CIR cells with a vector overexpressing human HLA-A2.

K562-A11 and K562-A24 cells were produced by retroviral transduction of K562 cells with vectors overexpressing human HLA-A11 and HLA-A24, respectively. Cells were cultured in DMEM + 10% FBS, RPMI + 10% FBS, or X-Vivo + 5% human serum A/B.

### Retroviral Vector Production

Retroviral vectors were prepared by transient transfection of HEK-293T cells using a standard calcium phosphate precipitation protocol. Viral supernatants were harvested at 48 hours and used to transduce T cells.

### Jurkat/T Cell Transduction and Expansion

Before retroviral transduction, PBMCs were activated for 2 days by culturing with T cell activator beads and human IL-2. For transduction, freshly harvested retroviral supernatant was spin-loaded onto non-tissue culture-treated 24-well plates coated with 15 µg RetroNectin per/well (Clontech Laboratories) by centrifuging 2 hours at 2,000 g at 32° C. Jurkat or activated PBMCs were loaded onto the plates and spun at 600 g at 32° C. for 30 minutes. Jurkat or T cells were incubated at 37° C. and 5% CO₂. Culture medium was replenished every 2 days.

### TCR Staining

All antibodies were obtained from BioLegend, Inc.. Expression of recombinant TCR was detected 48 hours after transduction by antibody staining to mouse TCR beta chain followed by flow cytometry. CD3 and/or CD8 staining were performed.

### CD69 Expression in TCR-Jurkat Cells

TCR-Jurkat cells were co-cultured with the indicated peptide pulsed APCs overnight, after which cell surface CD69 was measured by flow cytometry. Sorted CD3⁺ TCR⁺ cells were analyzed.

### In Vitro TCR-T IFN-γ Production

TCR-T cells were co-cultured overnight with the indicated peptide antigen-pulsed APCs (K562-A2, K562-A11 or K562-A24), after which intracellular IFN-y expression was measured by flow cytometry. Sorted CD3⁺ CD8⁺ cells were analyzed.

### EC50 Determination

TCR-T cells were co-cultured overnight with K562 cells overexpressing HLA-A11 or HLA-A24 and pulsed with increasing concentrations of LMP2 peptides. TCR-T cells and APCs were co-cultured at 1:1 effector-to-target ratios, after which T cells were collected and intracellular IFN-y expression was measured to determine EC50s.

### EXAMPLE 2. Sequences of Anti-LMP2 TCRs

Sequences of CDR1, CDR2 and CDR3 of the alpha chain variable domain and beta chain variable domain of various HLA-A2 typed anti-LMP2 TCRs are shown in FIG. 2. The HLA-A2 typed anti-LMP2 TCRs include L2-1, L2-2, L2-3, L2-4, L2-5, L2-6, L2-9, L2-10, L2-11, L2-12, L2-13, L2-16, L2-19, L2-23, L2-24, and L2-25 targeting amino acid 356-364 of the LMP2 protein. The HLA-A2 typed anti-LMP2 TCRs also include L208 targeting amino acid 426-434 of the LMP2 protein.

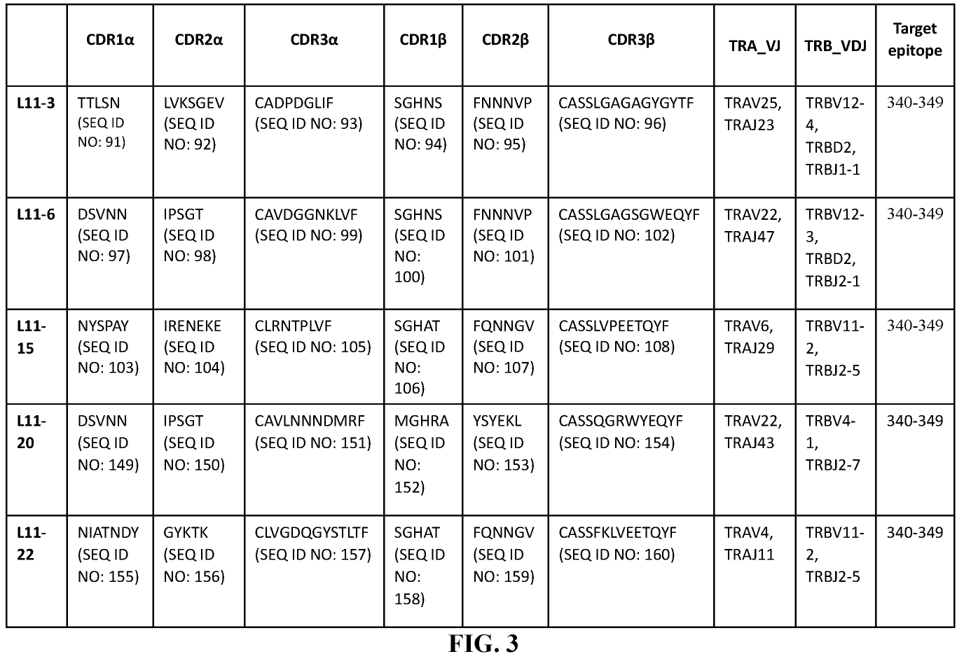

Sequences of CDR1, CDR2 and CDR3 of the alpha chain variable domain and beta chain variable domain of three HLA-A11 typed anti-LMP2 TCRs are shown in FIG. 3. The HLA-A11 typed anti-LMP2 TCRs include L11-3, L11-6, and L11-15 targeting amino acid 340-349 of the LMP2 protein. The HLA-A11 typed anti-LMP2 TCRs also include L11-20 and L11-22 targeting amino acid 340-349 of the LMP2 protein.

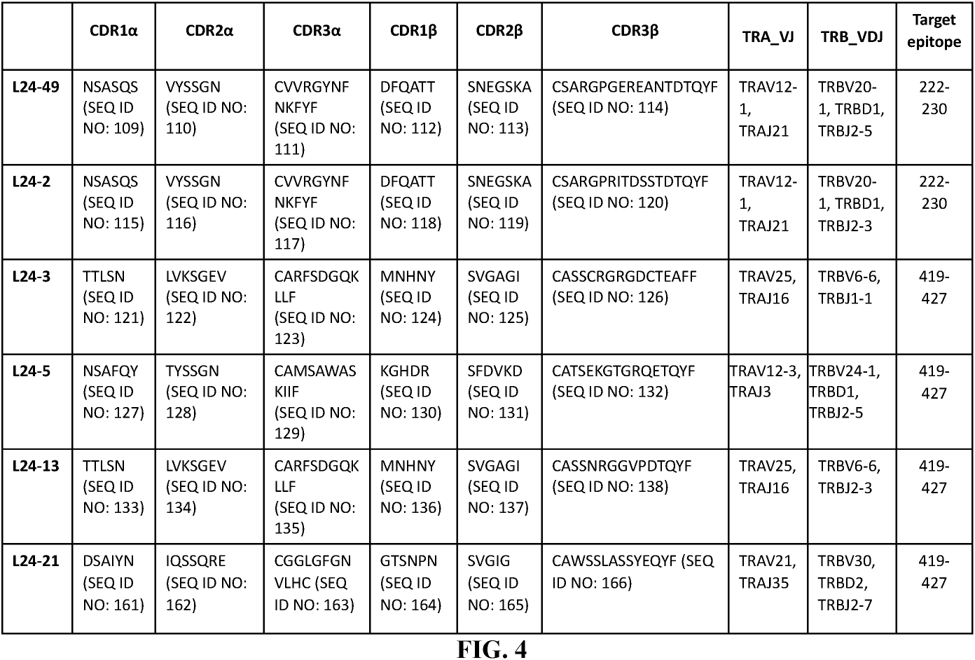

Sequences of CDR1, CDR2 and CDR3 of the alpha chain variable domain and beta chain variable domain of three HLA-A24 typed anti-LMP2 TCRs are shown in FIG. 4. The HLA-A24 typed anti-LMP2 TCRs include L24-49 and L24-2 targeting amino acid 222-230 of the LMP2 protein. The HLA-A24 typed anti-LMP2 TCRs also include L24-3, L24-5, L24-13, and L24-21 targeting amino acid 419-427 of the LMP2 protein.

### EXAMPLE 3. In Vitro Expression of HLA-A2 Typed Anti-LMP2 TCRs

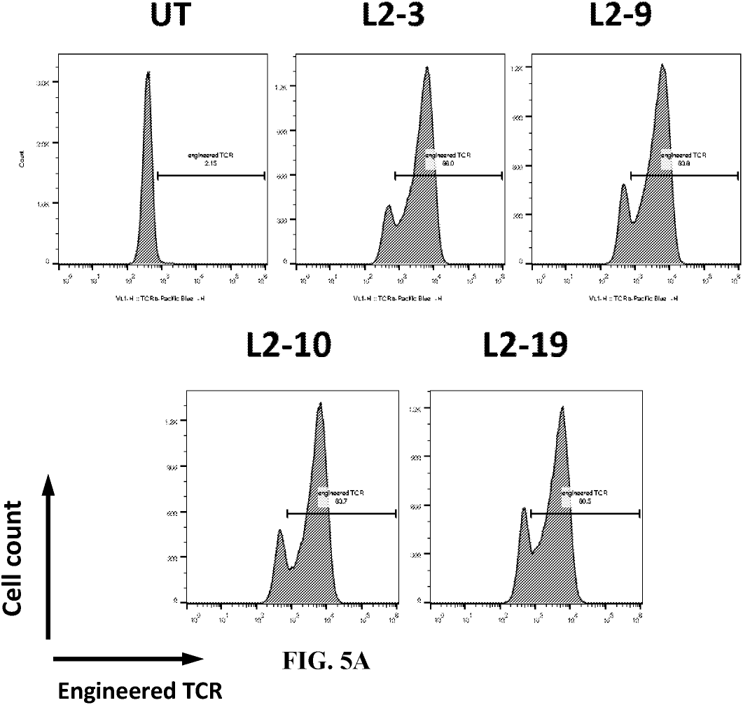

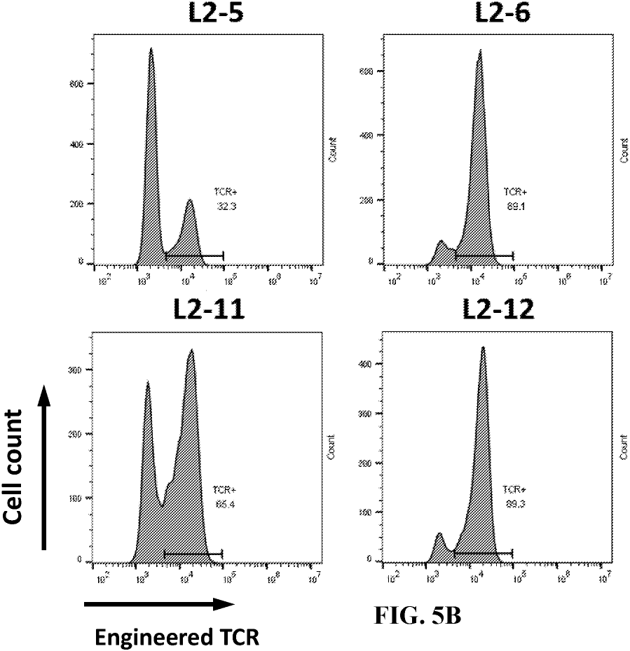

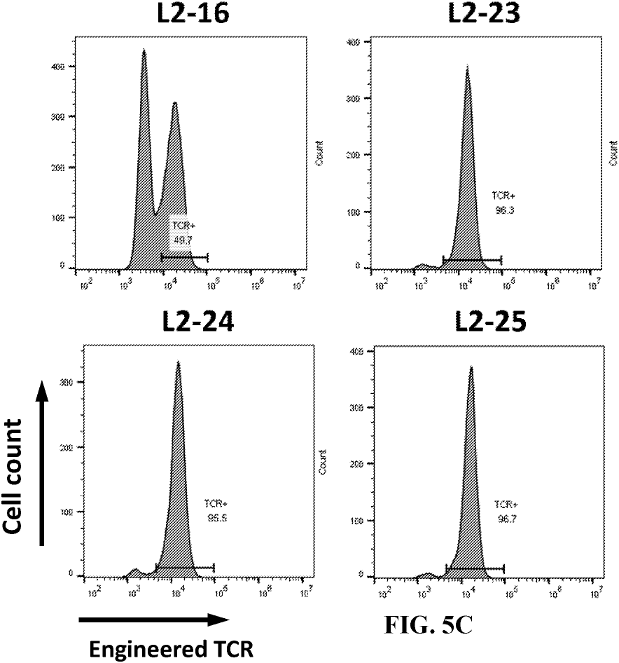

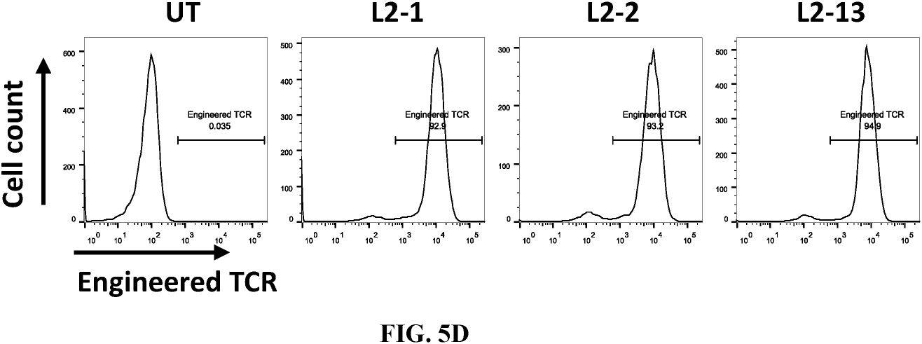

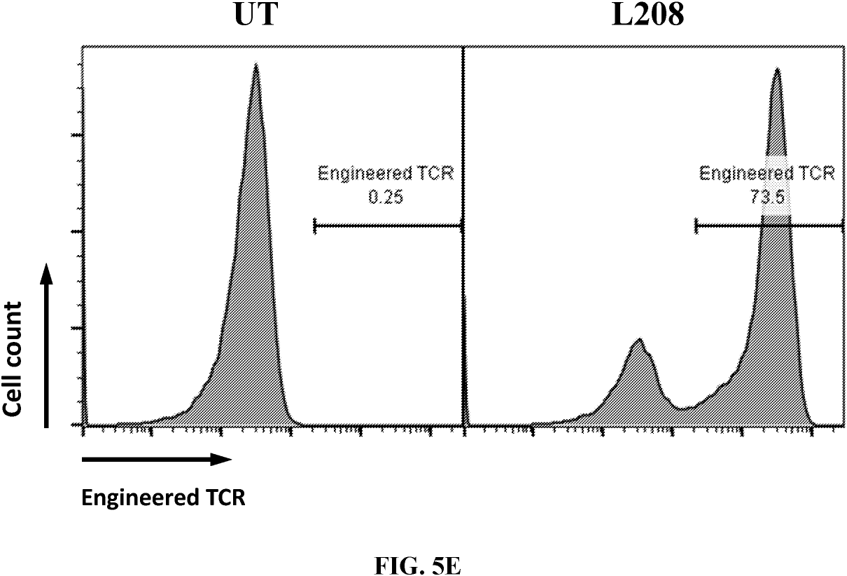

In vitro expression of HLA-A2 typed anti-LMP2 TCRs was measured by flow cytometry. As shown in FIGS. 5A-5C, Jurkat cells were either untransduced (UT), or transduced to express the L2-3, L2-9, L2-10, L2-19, L2-5, L2-6, L2-11, L2-12, L2-16, L2-23, L2-24, or L2-25 recombinant TCR. In a separate experiment as shown in FIG. 5D, primary human T cells were either untransduced (UT), or transduced to express the L2-1, L2-2, or L2-13 recombinant TCR. TCR expression was measured by staining mouse TCR beta chain 2 days after the transduction. Results in FIGS. 5A-5D indicate that the HLA-A2 typed anti-LMP2 TCRs were strongly expressed in human T cells.

In a separate experiment as shown in FIG. 5E, primary human T cells were either untransduced (UT), or transduced to express the L208 recombinant TCR. TCR expression was measured by staining mouse TCR beta chain 11 days after the transduction. Results in FIG. 5E indicate that the HLA-A2 typed anti-LMP2 TCR L208 was strongly expressed in human T cells.

### EXAMPLE 4. HLA-A2 Typed Anti-LMP2 TCR-T Cell Activation Upon Antigen-Specific Stimulation

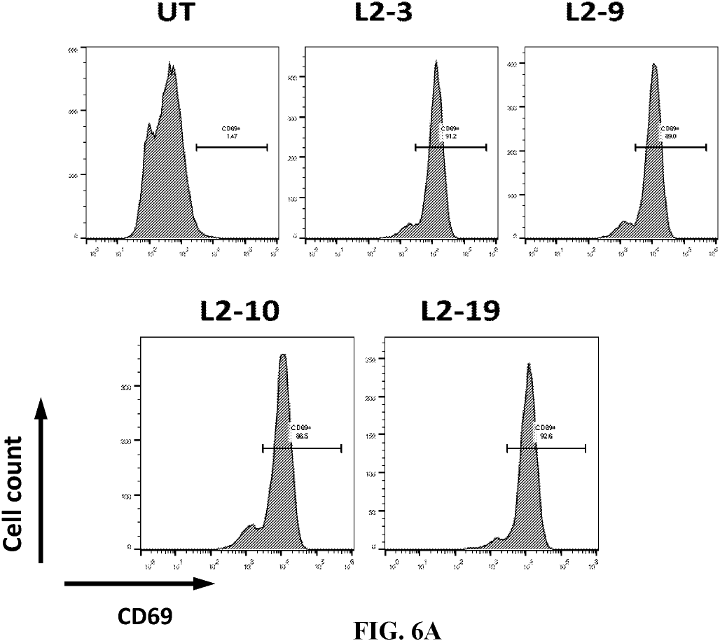

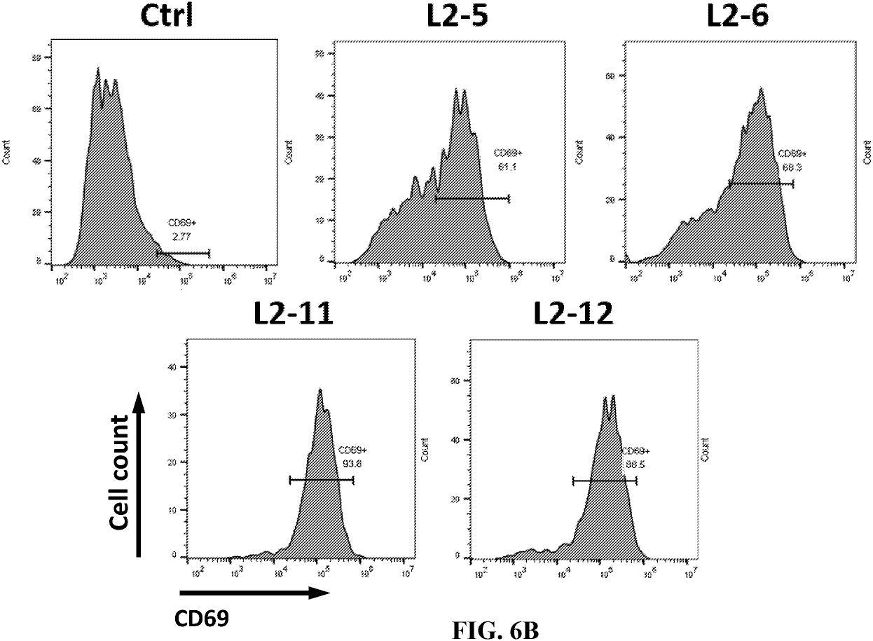

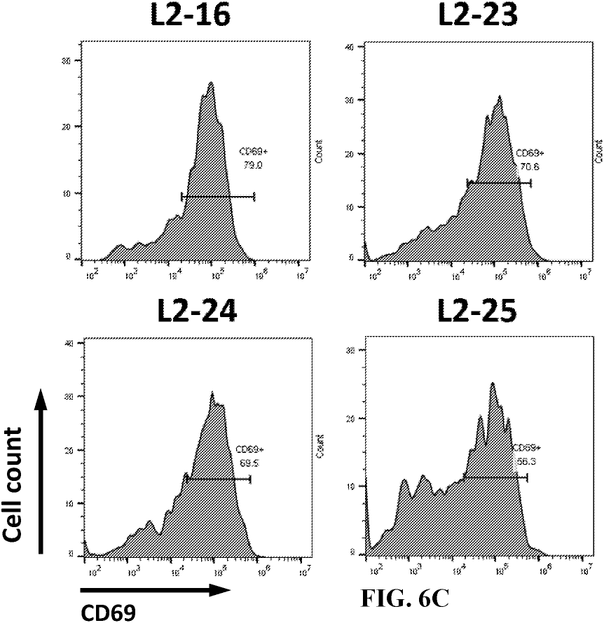

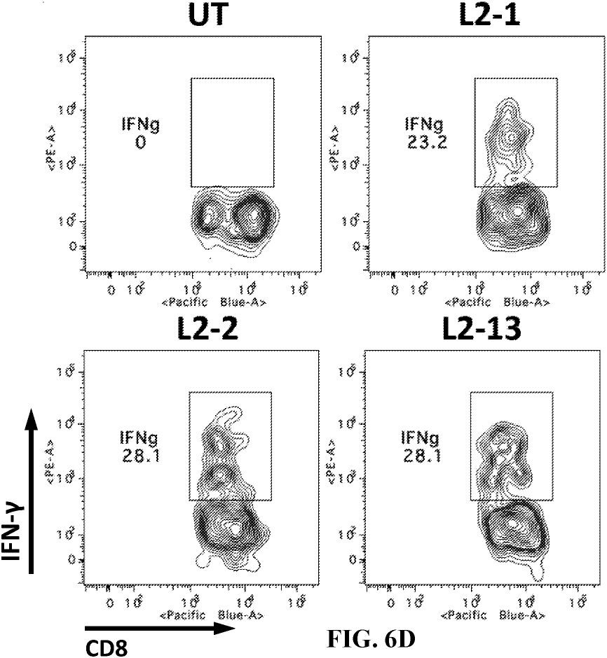

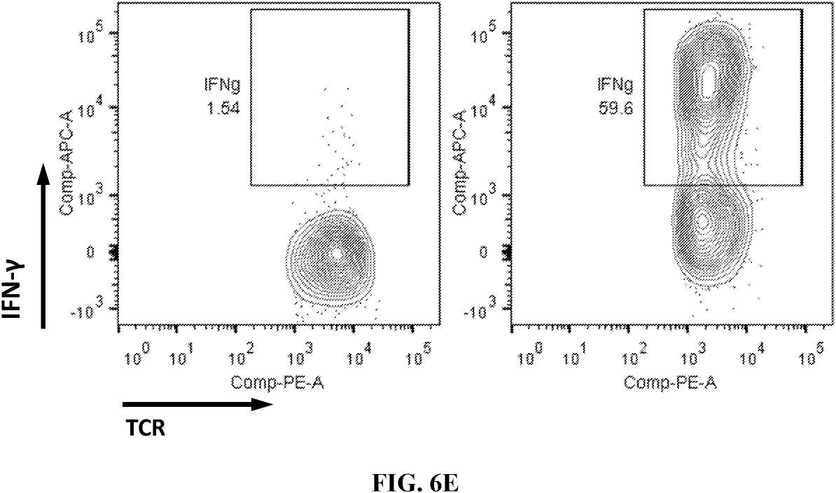

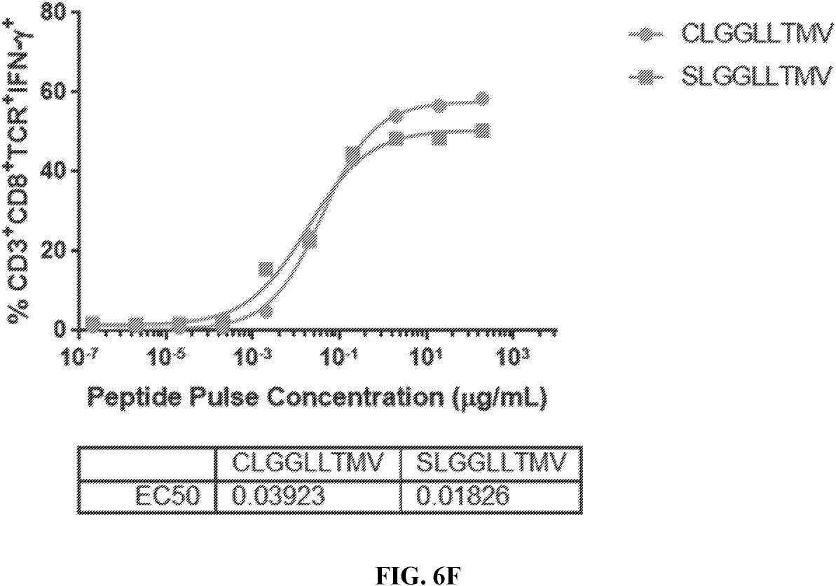

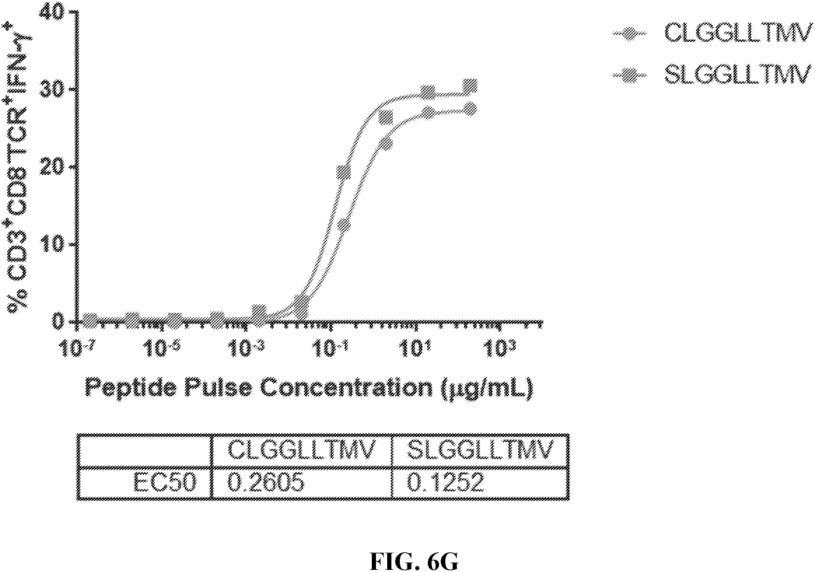

HLA-A2 typed anti-LMP2 TCR-T cell activation upon antigen-specific stimulation was determined by flow cytometry. As shown in FIGS. 6A-6C, untransduced (UT) Jurkat cells, or Jurkat cells transduced to express the L2-3, L2-9, L2-10, L2-19, L2-5, L2-6, L2-11, L2-12, L2-16, L2-23, L2-24 or L2-25 recombinant TCR were co-cultured with peptide FLYALALLL (or amino acid 356-364 of the LMP2 protein; SEQ ID NO: 139) pulsed antigen-presenting cells (APCs) overnight, after which surface CD69 expression was measured by flow cytometry. Results in FIGS. 6A-6C indicate that HLA-A2 typed TCR-T cells can be activated upon antigen-specific stimulation in vitro.

As shown in FIG. 6D, HLA-A2 typed anti-LMP2 TCR-T cell activation upon antigen-specific stimulation was also determined by measuring intracellular IFN-y expression in the activated TCR-T cells. More specifically, untransduced (UT) T cells, or TCR-T cells (PBMC T cells) expressing the L2-1, L2-2, or L2-13 recombinant TCR were co-cultured overnight with peptide FLYALALLL (SEQ ID NO: 139) pulsed APCs. T cells were then collected and intracellular IFN-y expression was measured by flow cytometry. Results in FIG. 6D indicate that TCR-T cells expressing HLA-A2 typed anti-LMP2 TCRs were specifically activated by cognate antigens, as measured by intracellular IFN-y expression.

In another experiment, untransduced (UT) T cells, or TCR-T cells (PBMC T cells) expressing the L208 recombinant TCR were co-cultured overnight with mutant peptide SLGGLLTMV (SEQ ID NO: 168) pulsed APCs. T cells were then collected and intracellular IFN-y expression was measured by flow cytometry. Results in FIG. 6E indicate that TCR-T cells expressing HLA-A2 typed anti-LMP2 TCR L208 were specifically activated by the mutant peptide from the LMP2 protein, as measured by intracellular IFN-y expression.

Half maximal effective concentration (EC50) of the cognate wild-type and mutant LMP2 peptide was determined. Specifically, TCR-T cells expressing the L208 recombinant TCR were co-cultured overnight with increasing concentrations of the wild-type LMP2 peptide CLGGLLTMV (SEQ ID NO: 167) or mutant LMP peptide SLGGLLTMV (SEQ ID NO: 168) pulsed APCs at 1:1 effector-to-target cell ratios. T cells were then collected and intracellular IFN-y expression was measured to determine EC50s. As shown in FIGS. 6F-6G, results showed that TCR-T cells expressing the HLA-A2 typed anti-LMP2 TCR L208 recognized APCs pulsed with the wild-type or mutant LMP2 peptides at EC50s below 1 µg/ml. For example, in one experiment (FIG. 6F), EC50 of the wild-type peptide was 0.03923 µg/ml, and EC50 of the mutant peptide was 0.01826 µg/ml. In a different experiment (FIG. 6G), EC50 of the wild-type peptide was 0.2605 µg/ml, and EC50 of the mutant peptide was 0.1252 µg/ml.

### EXAMPLE 5. In Vitro Expression of HLA-A11 Typed Anti-LMP2 TCRs

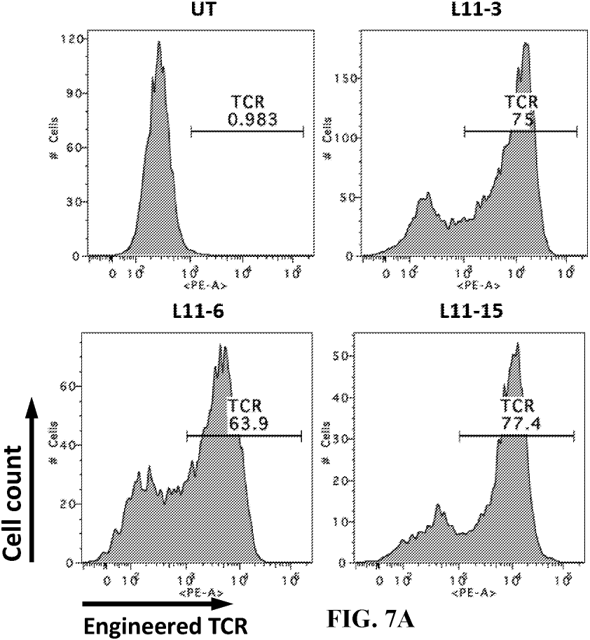

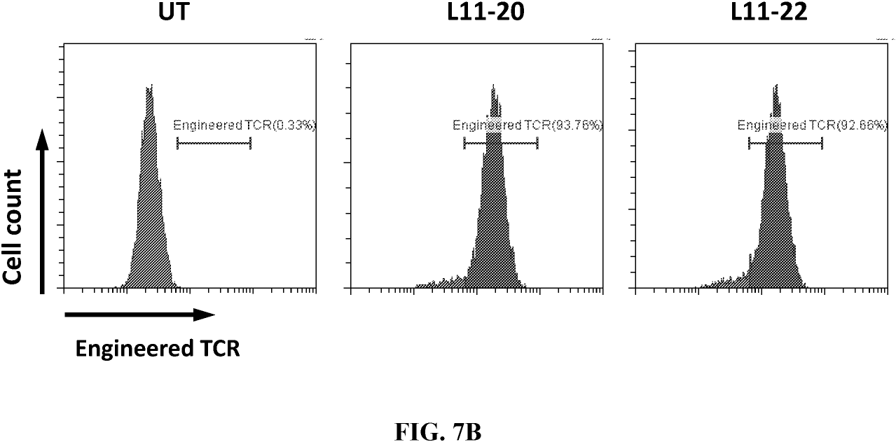

In vitro expression of HLA-A11 typed anti-LMP2 TCRs was measured by flow cytometry. As shown in FIGS. 7A-7B, primary T cells were either untransduced (UT), or transduced to express the L11-3, L11-6, L11-15, L11-20, or L11-22 recombinant TCR. TCR expression was measured by staining mouse TCR beta chain 2 days after the transduction. Results in FIGS. 7A-7B indicate that the HLA-A11 typed anti-LMP2 TCRs were strongly expressed in human T cells.

### EXAMPLE 6. HLA-A11 Typed Anti-LMP2 TCR-T Cell Activation Upon Antigen-specific stimulation

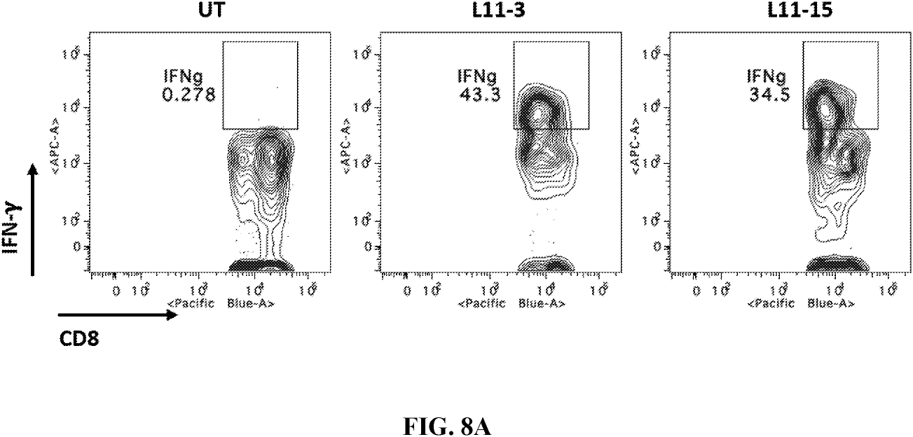

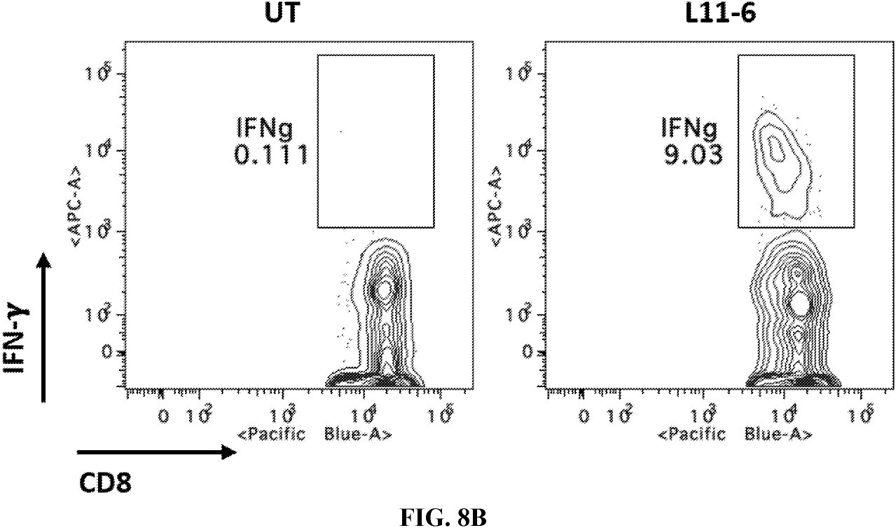

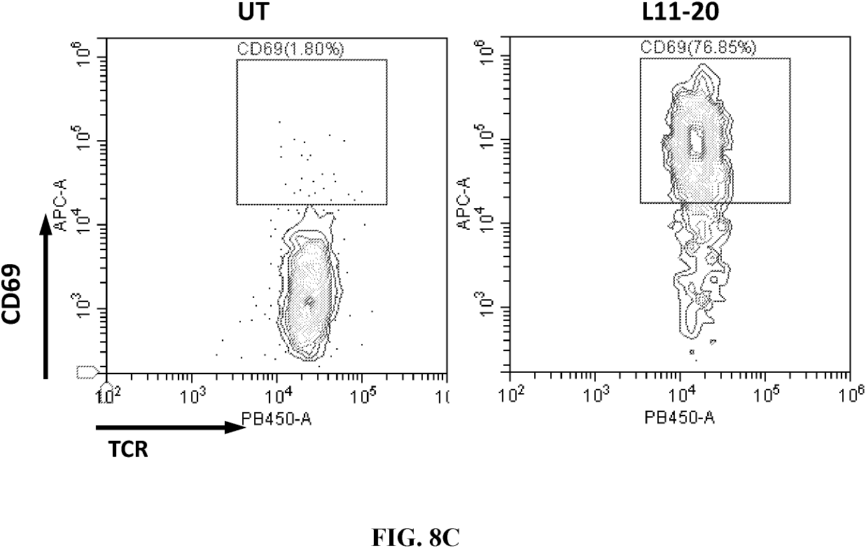

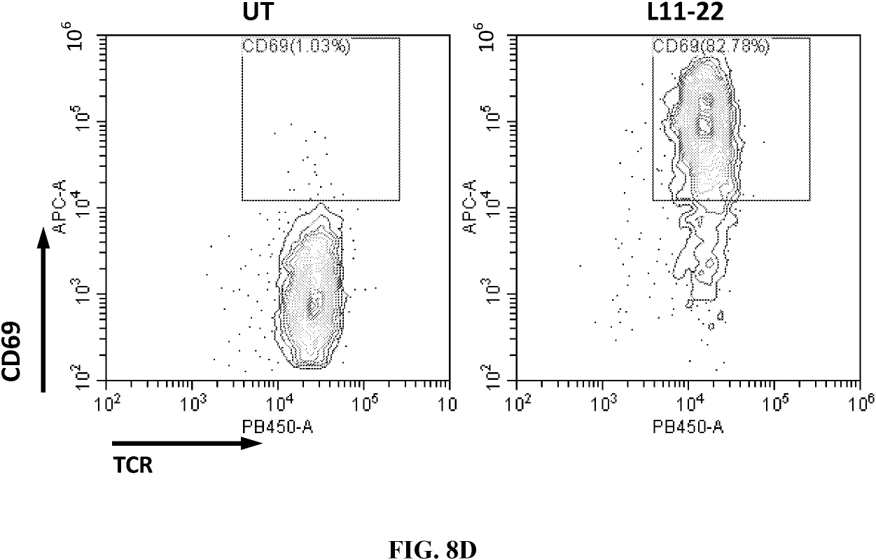

HLA-A11 typed anti-LMP2 TCR-T cell activation upon antigen-specific stimulation was determined by flow cytometry. Experiments were performed as shown in FIGS. 8A-8B, in which HLA-A11 typed anti-LMP2 TCR-T cell activation upon antigen-specific stimulation was determined by measuring intracellular IFN-y expression in the activated TCR-T cells. More specifically, untransduced (UT) T cells, or TCR-T cells (PBMC T cells) expressing the L11-3, L11-15, or L11-6 recombinant TCR were co-cultured overnight with peptide SSCSSCPLSK (or amino acid 340-349 of the LMP2 protein; SEQ ID NO: 140) pulsed APCs. T cells were then collected and intracellular IFN-y expression was measured by flow cytometry.

In FIGS. 8C-8D, untransduced (UT) T cells, or TCR-T cells (PBMC T cells) expressing the L11-20 or L11-22 recombinant TCR were co-cultured overnight with peptide SSCSSCPLSK (or amino acid 340-349 of the LMP2 protein; SEQ ID NO: 140) pulsed APCs, after which surface CD69 expression was measured by flow cytometry.

Results in FIGS. 8A-8D indicate that TCR-T cells expressing HLA-A11 typed anti-LMP2 TCRs were specifically activated by cognate antigens, as measured by intracellular IFN-y expression and CD69.

### EXAMPLE 7. EC50s of HLA-A11 Typed Anti-LMP2 TCRs

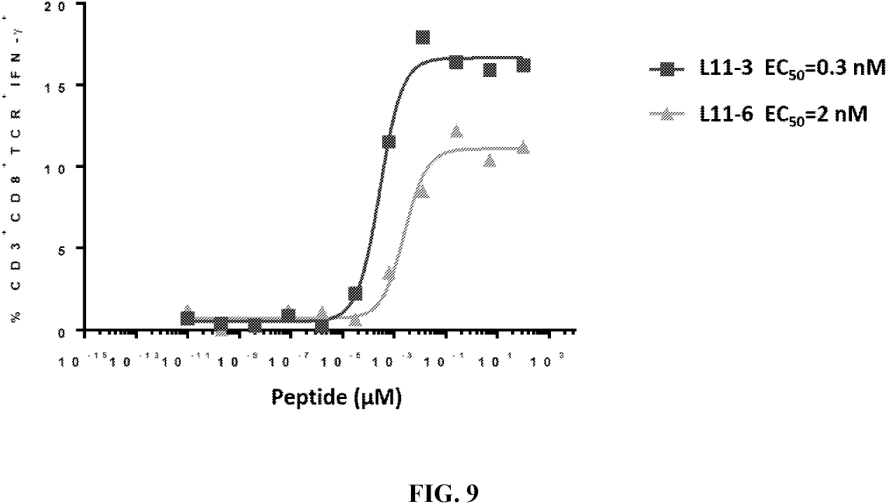

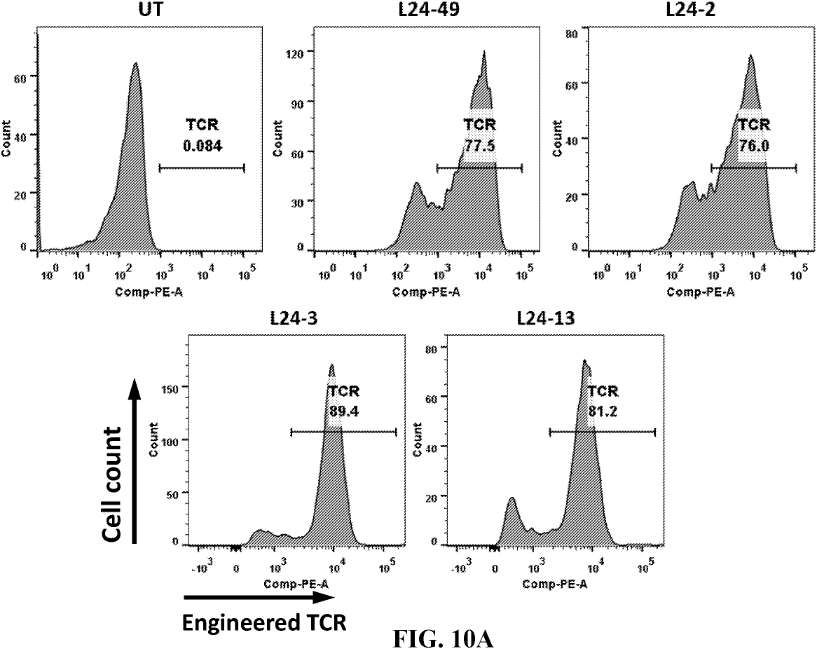

Half maximal effective concentration (EC50) of the cognate LMP2 peptide was determined. Specifically, TCR-T cells expressing the L11-3 or L11-6 recombinant TCR were co-cultured overnight with increasing concentrations of the LMP2 peptide SSCSSCPLSK (SEQ ID NO: 140) pulsed APCs at 1:1 effector-to-target cell ratios. T cells were then collected and intracellular IFN-y expression was measured to determine EC50s. As shown in FIG. 9, results showed that TCR-T cells expressing the HLA-A11 typed anti-LMP2 TCRs recognized APCs pulsed with LMP2 peptide at EC50s in the nM-to-µM range. For example, EC50 for L11-3 is 0.3 nM, and EC50 for L11-6 is 2 nM.

### EXAMPLE 8. In Vitro Expression of HLA-A24 Typed Anti-LMP2 TCRs

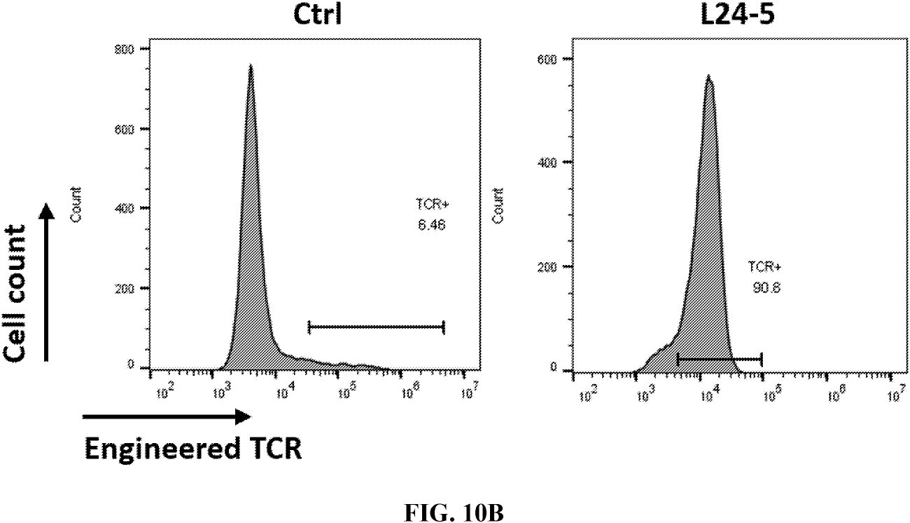

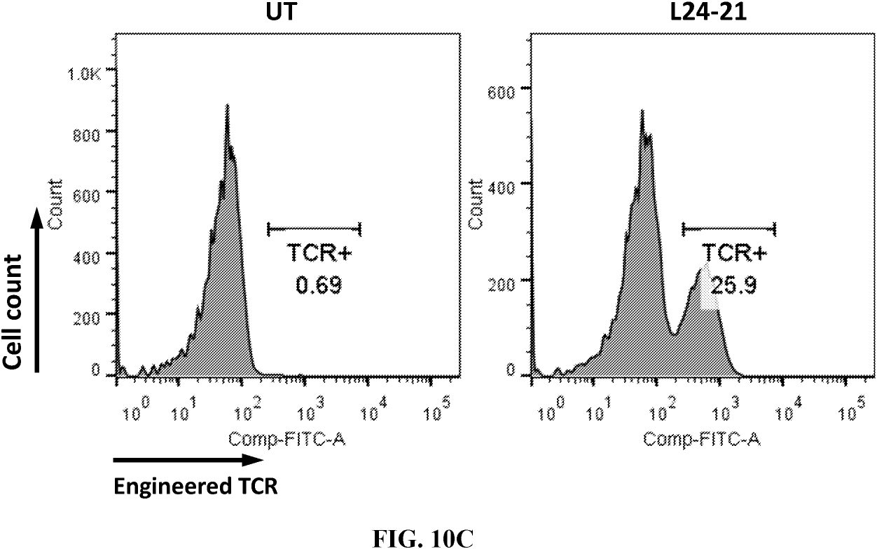

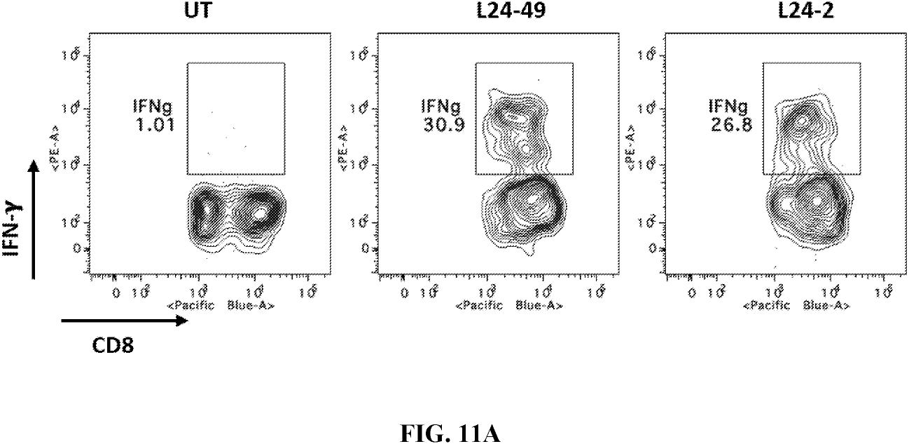

In vitro expression of HLA-A24 typed anti-LMP2 TCRs was measured by flow cytometry. As shown in FIG. 10A and FIG. 10C, primary T cells were either untransduced (UT), or transduced to express the L24-49, L24-2, L24-3, L24-13, L24-21 recombinant TCR. In a separate experiment as shown in FIG. 10B, Jurkat cells were transduced with an empty vector or vector encoding the L24-5 recombinant TCR. TCR expression was measured by staining mouse TCR beta chain 2 days after the transduction.

Results in FIGS. 10A-10C indicate that the HLA-A24 typed anti-LMP2 TCRs were strongly expressed in human T cells.

### EXAMPLE 9. HLA-A24 Typed Anti-LMP2 TCR-T Cell Activation Upon Antigen-Specific Stimulation

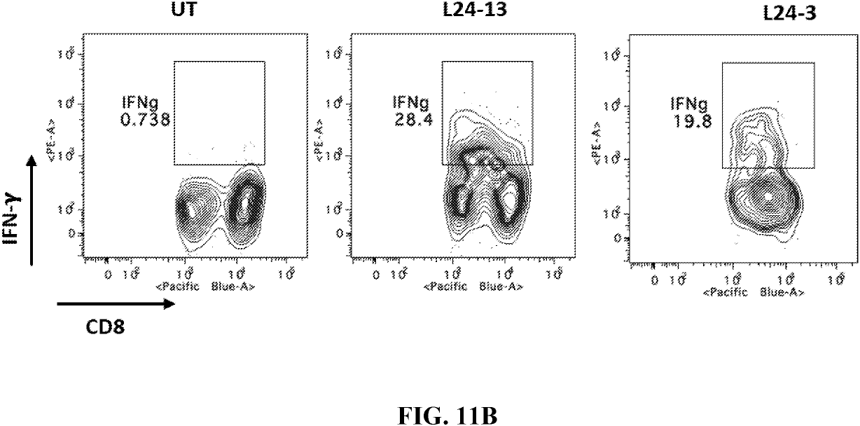

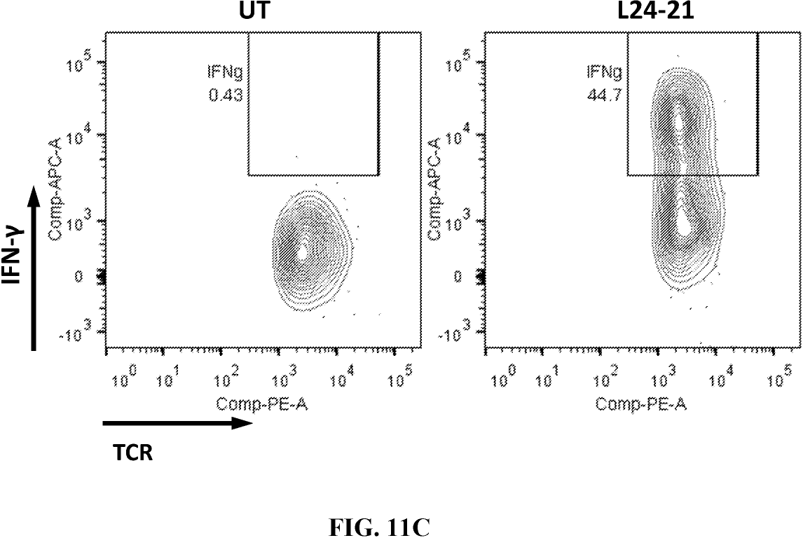

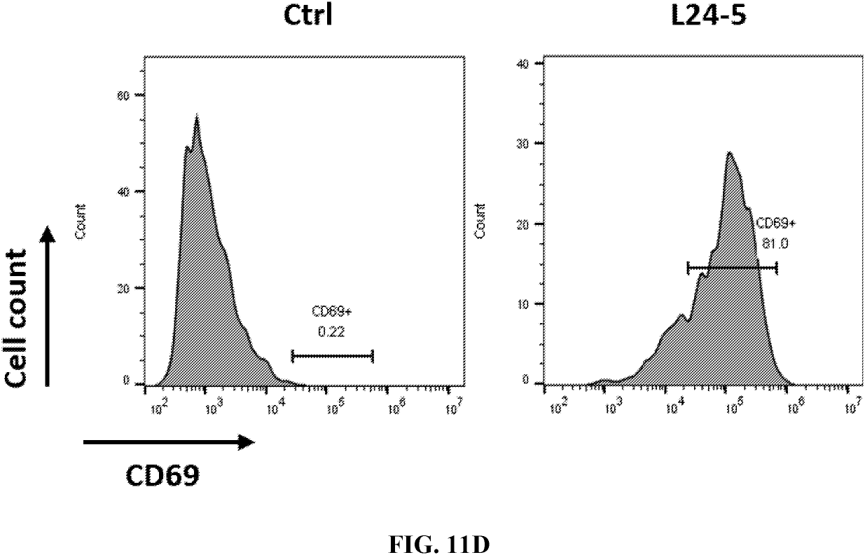

HLA-A24 typed anti-LMP2 TCR-T cell activation upon antigen-specific stimulation was determined by flow cytometry. As shown in FIGS. 11A-11B, HLA-A24 typed anti-LMP2 TCR-T cell activation upon antigen-specific stimulation was determined by measuring intracellular IFN-y expression in the activated TCR-T cells. More specifically, untransduced (UT) T cells, or TCR-T cells (PBMC T cells) expressing the L24-49 or L24-2 recombinant TCR were co-cultured overnight with peptide IYVLVMLVL (or amino acid 222-230 of the LMP2 protein; SEQ ID NO: 141) pulsed APCs (FIG. 11A). In a separate experiment, untransduced (UT) T cells, or TCR-T cells expressing the L24-3 or L24-13 recombinant TCR were co-cultured overnight with peptide TYGPVFMCL (or amino acid 419-427 of the LMP2 protein; SEQ ID NO: 142) pulsed APCs (FIG. 11B). In a separate experiment, untransduced (UT) T cells, or TCR-T cells expressing the L24-21 recombinant TCR were co-cultured overnight with peptide TYGPVFMCL (or amino acid 419-427 of the LMP2 protein; SEQ ID NO: 142) pulsed APCs (FIG. 11C). T cells were then collected and intracellular IFN-y expression was measured by flow cytometry. Results in FIGS. 11A-11C indicate that TCR-T cells expressing HLA-A24 typed anti-LMP2 TCRs were specifically activated by cognate antigens, as measured by intracellular IFN-y expression.

As shown in FIG. 11D, control (ctrl) Jurkat cells, or Jurkat cells transduced to express the L24-5 recombinant TCR were co-cultured with peptide TYGPVFMCL (SEQ ID NO: 142) pulsed antigen-presenting cells (APCs) overnight, after which surface CD69 expression was measured by flow cytometry. Results in FIG. 11D indicate that HLA-A24 typed TCR-T cells can be activated upon antigen-specific stimulation in vitro.

### EXAMPLE 10. EC50s of HLA-A24 Typed Anti-LMP2 TCRs

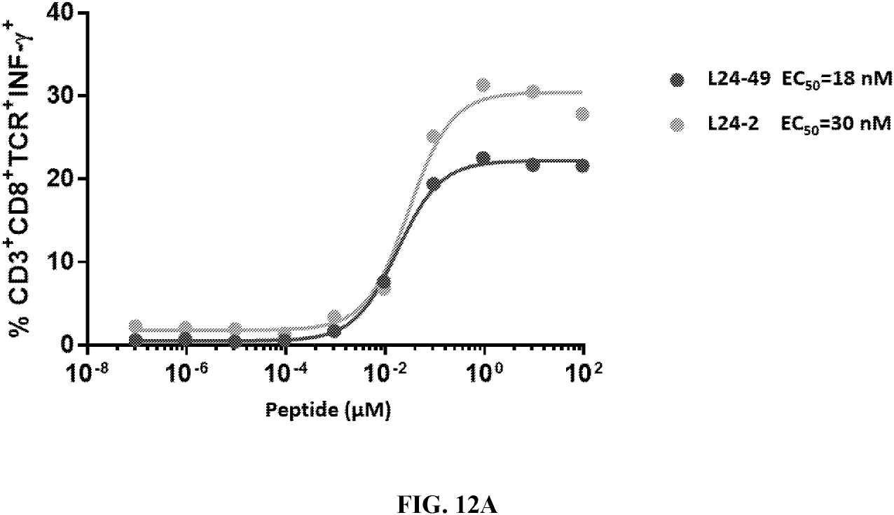

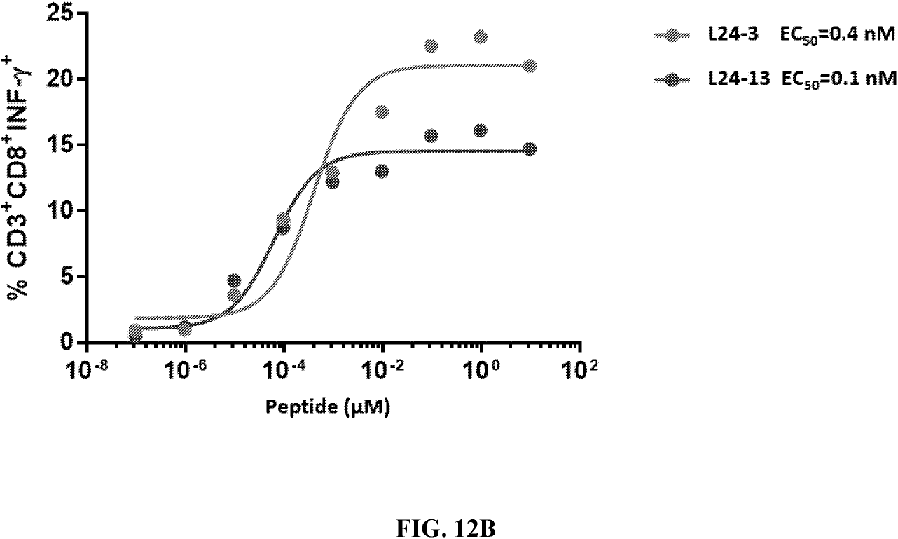

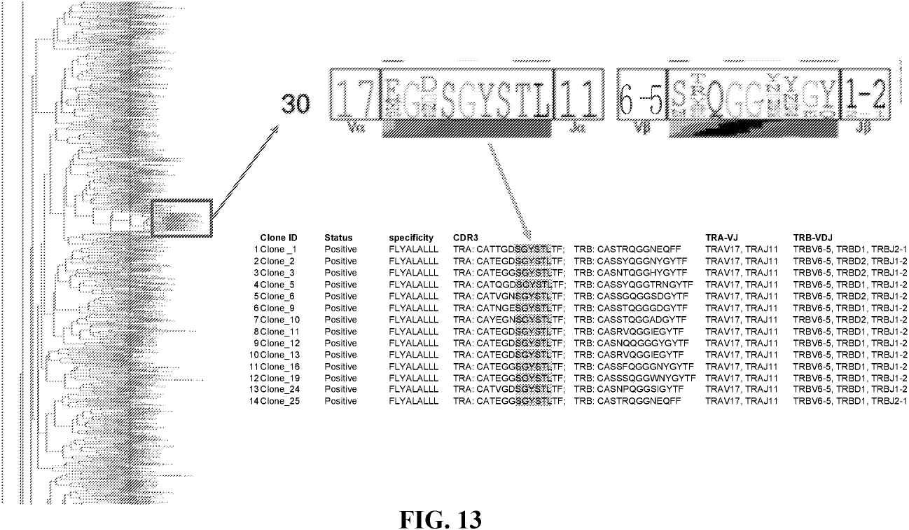

Half maximal effective concentration (EC50) of the cognate LMP2 peptide was determined. Specifically, TCR-T cells expressing the L24-49 or L24-2 recombinant TCR were co-cultured overnight with increasing concentrations of the LMP2 peptide IYVLVMLVL (SEQ ID NO: 141) pulsed APCs at 1:1 effector-to-target cell ratios. In a separate experiment, TCR-T cells expressing the L24-3 or L24-13 recombinant TCR were co-cultured overnight with increasing concentrations of the LMP2 peptide TYGPVFMCL (SEQ ID NO: 142) pulsed APCs at 1:1 effector-to-target cell ratios. T cells were then collected and intracellular IFN-y expression was measured to determine EC50s. As shown in FIGS. 12A-12B, results showed that TCR-T cells expressing the HLA-A24 typed anti-LMP2 TCRs recognized APCs pulsed with LMP2 peptide at EC50s in the nM-to-µM range.

### EXAMPLE 11. Motif Analysis on EBV Positive TCRs

Motifs that are highly enriched in EBV positive TCRs were analyzed. Methods used to find the most important motifs include TCRdist, Support Vector Machine (SVM), Fisher exact Test. Once the motif were determined, the Cramer’s V test was applied to measure association strength between motif and outcome.

First, TCRdist algorithm was utilized to find the most significantly enriched motifs. Specifically, the distance of a minimum unit was measured by editing distance between two amino acids. Domain-based analysis was used to measure distances of different domains, such that a CDR2.5 (α/β) domain was defined in the scoring system. The TCR alpha chain (TRA) and TCR beta chain (TRB) were integrated for measuring the distance between clones through summing weighted score in different domains of the TCR complex. Details of the TCRdist algorithm can be found, e.g., in Dash et al., “Quantifiable predictive features define epitope-specific T cell receptor repertoires.” Nature 547.7661 (2017): 89-93, which is incorporated herein by reference in its entirety. The TCRdist analysis result is shown in FIG. 13. The clones in the indicated cluster was significantly closer (similar) to each other, compared with the clones in all other clusters.

A total of 30 TCR clones were identified in the cluster and 14 were tested as positive clones by experiments. These positive clones can recognize the target epitope and activate T cells. Two conserved motifs were identified among the positive clones, which were “SGYSTL” within CDR3 of TRA and “QGG” within CDR3 of TRB.

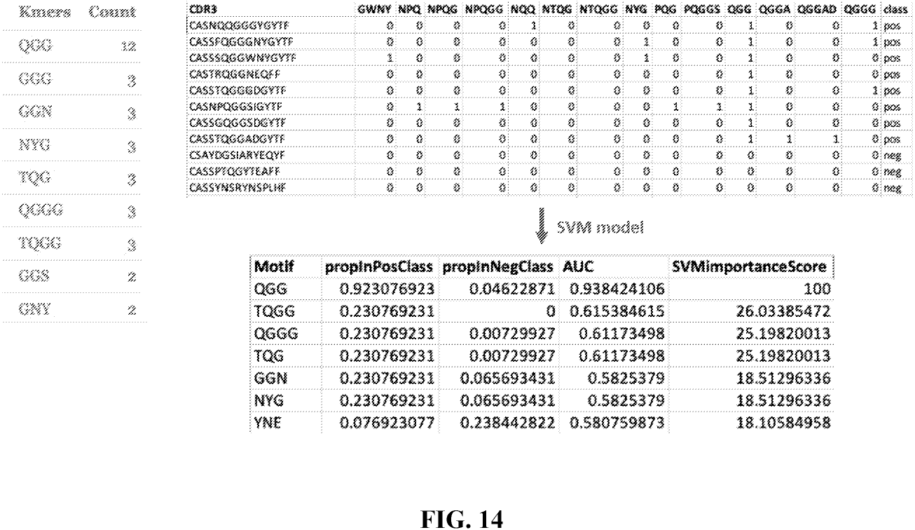

Second, Support Vector Machine (SVM) was utilized to find the most significant motifs. Specifically, before running SVM, motifs of 3 mers to 5 mers were generated using R package tcR. An SVM model with cross validation was run on the “positive” EBV TCR motifs and “negative” HPV single cell TCRs. This model was run with class weight/cost sensitive learning. Cost-sensitive learning took the costs of prediction errors into account when training a machine learning model. SVM was utilized to provide important variables in predicting the outcome. These high accuracy variables were then used to obtain area under the curve (AUC) values and ROC curve. As shown in FIG. 14, the SVM analysis result shows the top ranked motifs as “QGG” and “TQGG”.

Third, Fisher exact test (or Fisher’s exact test) and Cramer’s V test were performed. Fisher’s exact test is a way to test the association between two categorical variables for small cell sizes (expected values less than 5). P value from Fisher’s exact test were adjusted using the FDR method. Adjusted P value of less than 0.05 was considered significant. Cramer’s V statistic is another test to measure of the relative (strength) of an association between two categorical variables. The table below describes the value of Cramer V and its association.

TABLE 6

| | |
|---|---|
| >0.5 | high association |
| 0.3 to 0.5 | moderate association |
| 0.1 to 0.3 | low association |
| 0 to 0.1 | little if any association |

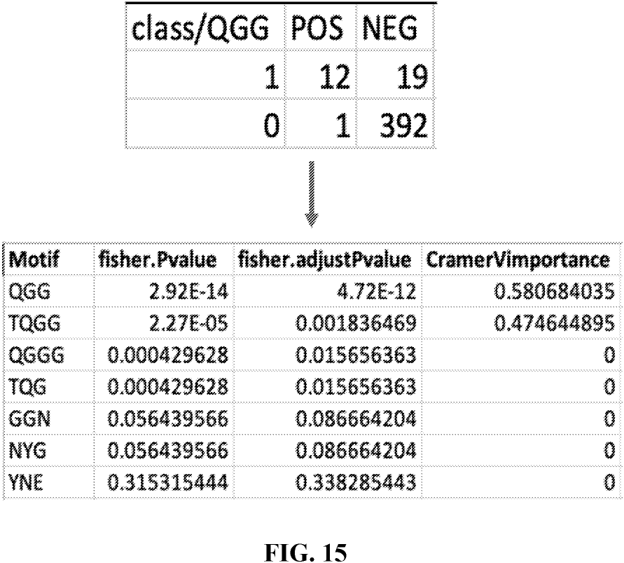

The Fisher exact and Cramer’s V test results are shown in FIG. 15. P values, adjusted P values by the Fisher exact test, and Cramer importance scores by the Cramer’s V test are listed for the top ranked motifs, including “QGG” and “TQGG”.

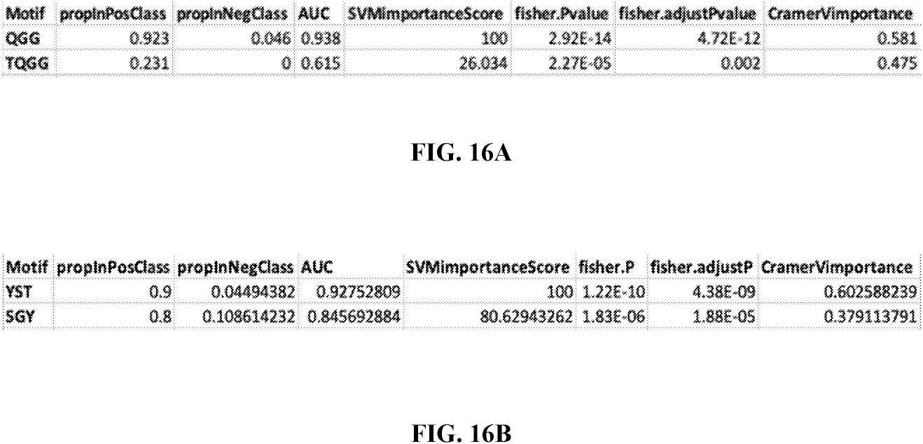

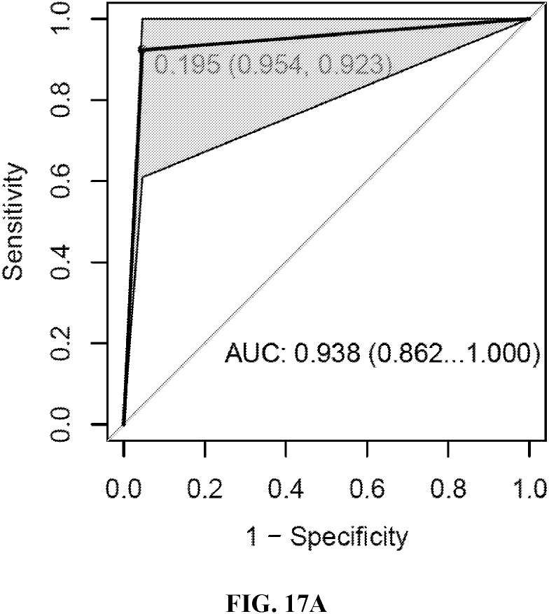

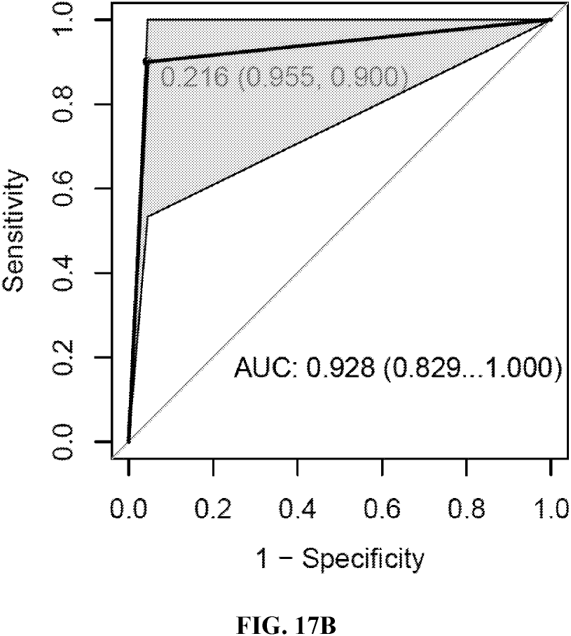

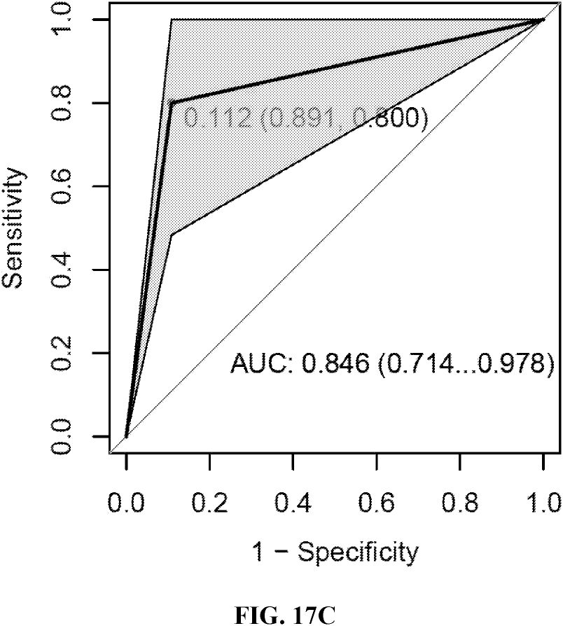

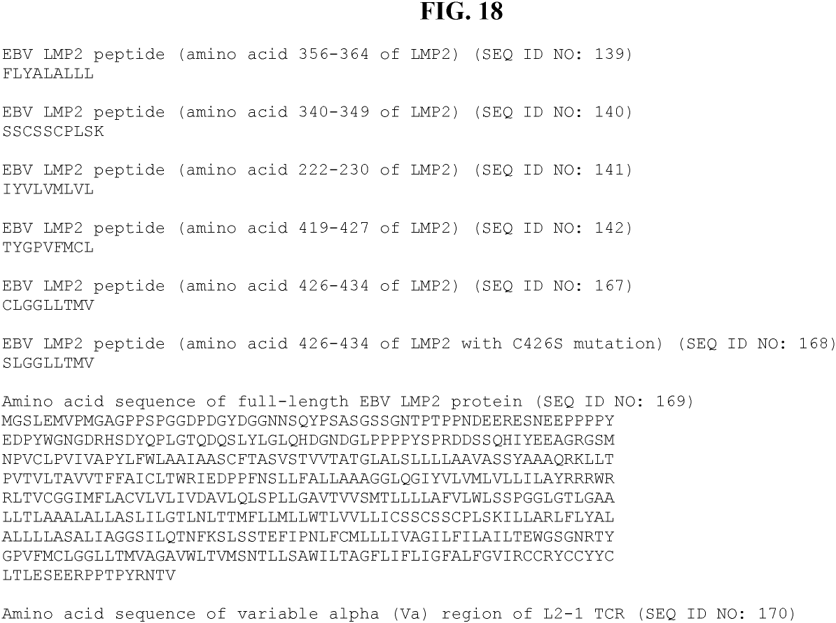

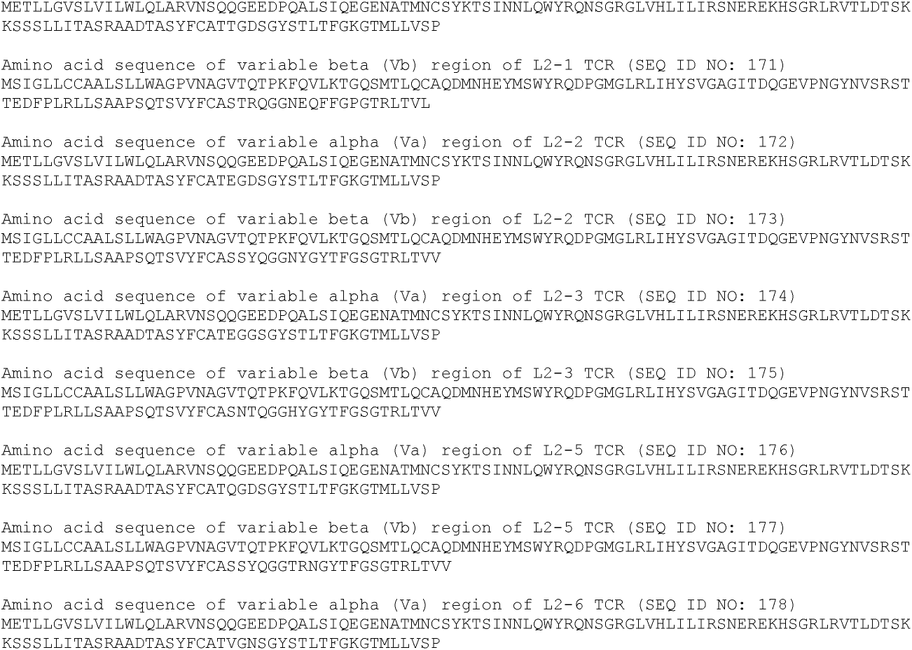

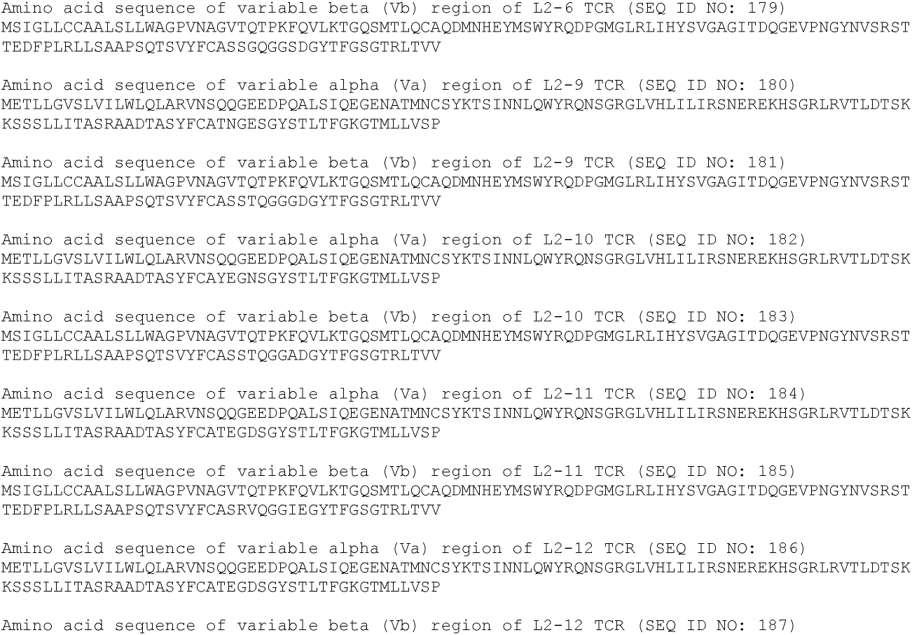

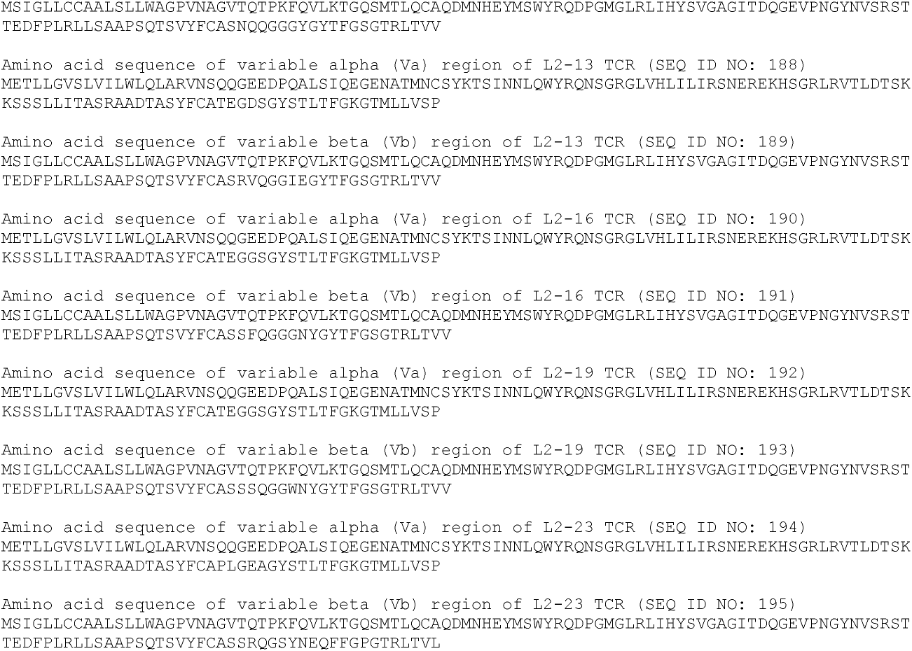

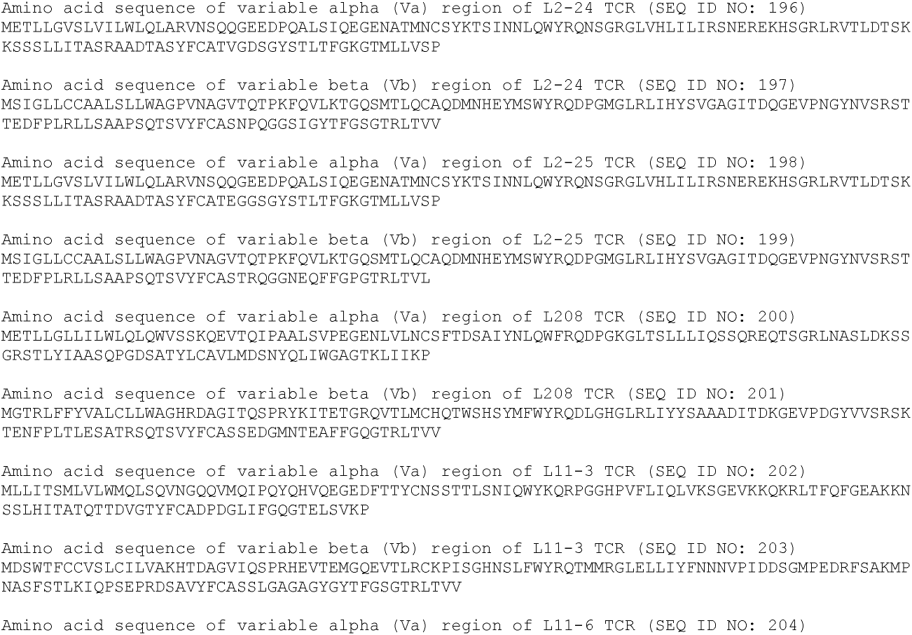

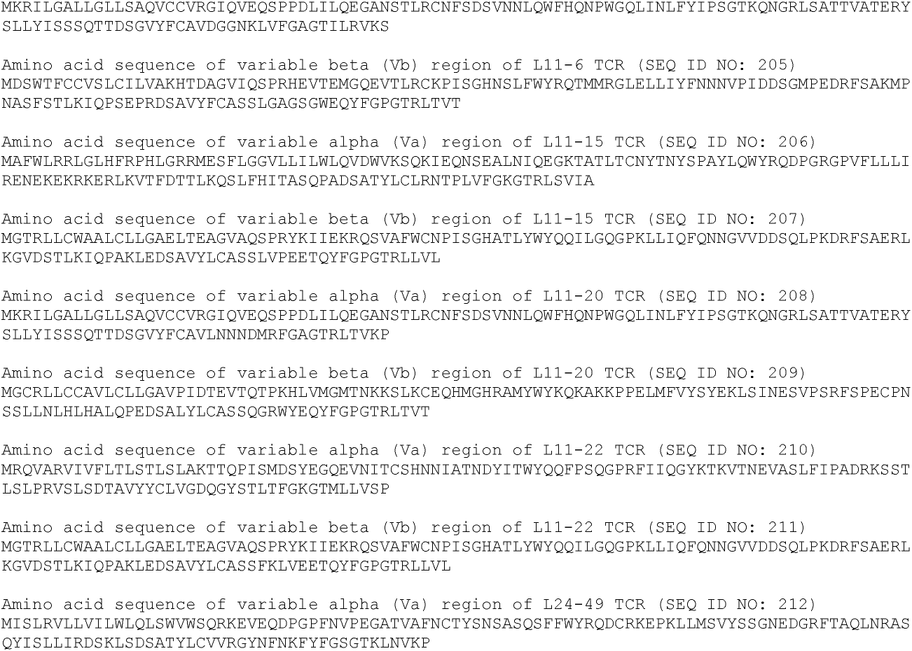

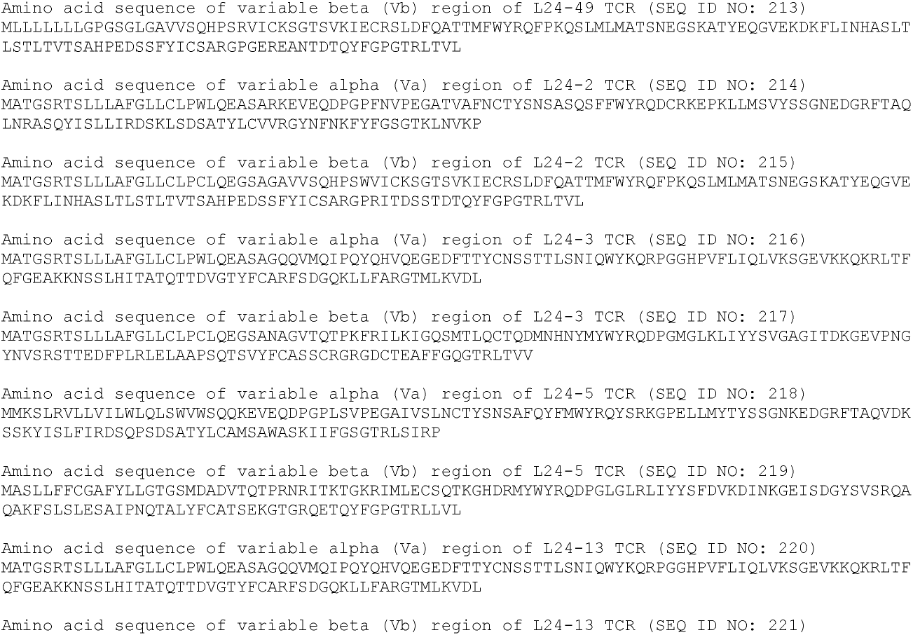

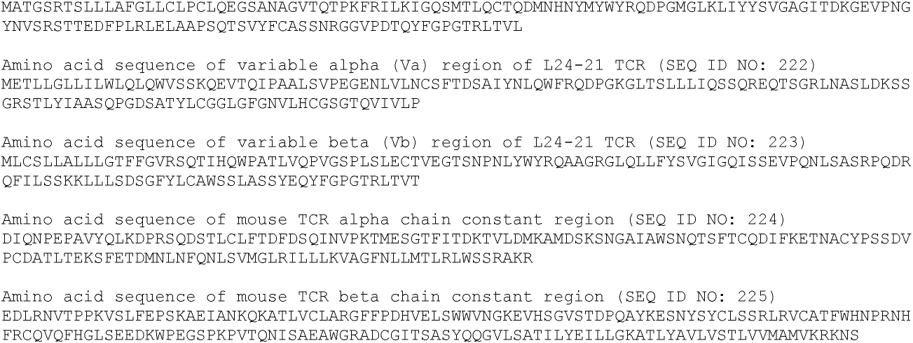

In summary, motifs that pass in all the above methods are considered as the most significant markers in positive TCRs, as shown in FIGS. 16A-16B. Within CDR3 of the TCR beta chain from the EBV-A02 dataset, motif “QGG” was highly enriched. Within CDR3 of the TCR alpha chain from the EBV-A02 dataset, motifs “YST” and “SGY” were highly enriched. AUC values of motifs “QGG”, “YST” and “SGY” were calculated as shown in FIGS. 17A-17C.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A T cell receptor (TCR) or antigen-binding fragment thereof, comprising an alpha chain comprising a variable alpha (Va) region and a beta chain comprising a variable beta (Vb) region, wherein
(a) the Va region comprises a complementarity determining region 1 (CDR1), a complementarity determining region 2 (CDR2), and a complementarity determining region 3 (CDR3), wherein the CDR3 of the Va region comprises an amino acid sequence X₁GX₂SGYSTL, wherein
the X₁ is a E, T, Q, V, or N,
the X₂ is a D, G, N, or E ; and
(b) the Vb region comprises a CDR1, a CDR2, and a CDR3, wherein the CDR3 of the Vb region comprises an amino acid sequence X₃X₄QGGX₅X₆X₇X₈, wherein
the X₃ is a S, T, N, or R,
the X₄ is a T, R, Y, G, V, Q, F, S, or P,
the X₅ is a N, G, H, T, S, A, I, or W,
the X₆ is a Y, N, D, E, R, or I,
the X₇ is a G, Q, N, Y,
the X₈ is a Y, F, or G.

2. The T cell receptor (TCR) or antigen-binding fragment thereof of claim 1, wherein the Va region is encoded by a sequence from rearrangement of a human TRAV gene segment and a human TRAJ gene segment, and the Vb region is encoded by a sequence from rearrangement of a human TRBV gene segment, optionally a human TRBD gene segment, and a human TRBJ gene segment, wherein
the TRAV gene segment is TRAV17;
the TRAJ gene segment is TRAJ11;
the TRBV gene segment is TRBV6-5;
the TRBD gene segment is TRBD1 or TRBD2; and
the TRBJ gene segment is TRBJ2-1 or TRBJ1-2.

3. The T cell receptor (TCR) or antigen-binding fragment thereof of claim 1 or claim 2, wherein the Va CDR1 region comprises an amino acid sequence that is at least 80% identical to TSINN (SEQ ID NO: 1), the Va CDR2 region comprises an amino acid sequence that is at least 80% identical to IRSNERE (SEQ ID NO: 2), the Vb CDR1 region comprises an amino acid sequence that is at least 80% identical to MNHEY (SEQ ID NO: 4), and the Vb CDR2 region comprises an amino acid sequence that is at least 80% identical to SVGAGI (SEQ ID NO: 5).

4. The T cell receptor (TCR) or antigen-binding fragment thereof of any one of claims 1-3,
wherein the Va CDR1 region comprises an amino acid sequence that is at least 80% identical to a selected Va CDR1 amino acid sequence, the Va CDR2 region comprises an amino acid sequence that is at least 80% identical to a selected Va CDR2 amino acid sequence, the Va CDR3 region comprises an amino acid sequence that is at least 80% identical to a selected Va CDR3 amino acid sequence; and
wherein the Vb CDR1 region comprises an amino acid sequence that is at least 80% identical to a selected Vb CDR1 amino acid sequence, the Vb CDR2 region comprises an amino acid sequence that is at least 80% identical to a selected Vb CDR2 amino acid sequence, the Vb CDR3 region comprises an amino acid sequence that is at least 80% identical to a selected Vb CDR3 amino acid sequence;
wherein the selected Va CDRs 1, 2, and 3 and Vb CDRs 1, 2, and 3 amino acid sequences are one of the following:
(1) the selected Va CDRs 1, 2, 3 amino acid sequences are set forth in SEQ ID NOs: 1-3, respectively, and the selected Vb CDRs 1, 2, 3 amino acid sequences are set forth in SEQ ID NOs: 4-6, respectively;
(2) the selected Va CDRs 1, 2, 3 amino acid sequences are set forth in SEQ ID NOs: 7-9, respectively, and the selected Vb CDRs 1, 2, 3 amino acid sequences are set forth in SEQ ID NOs: 10-12, respectively;
(3) the selected Va CDRs 1, 2, 3 amino acid sequences are set forth in SEQ ID NOs: 13-15, respectively, and the selected Vb CDRs 1, 2, 3 amino acid sequences are set forth in SEQ ID NOs: 16-18, respectively;
(4) the selected Va CDRs 1, 2, 3 amino acid sequences are set forth in SEQ ID NOs: 19-21, respectively, and the selected Vb CDRs 1, 2, 3 amino acid sequences are set forth in SEQ ID NOs: 22-24, respectively;
(5) the selected Va CDRs 1, 2, 3 amino acid sequences are set forth in SEQ ID NOs: 25-27, respectively, and the selected Vb CDRs 1, 2, 3 amino acid sequences are set forth in SEQ ID NOs: 28-30, respectively;
(6) the selected Va CDRs 1, 2, 3 amino acid sequences are set forth in SEQ ID NOs: 31-33, respectively, and the selected Vb CDRs 1, 2, 3 amino acid sequences are set forth in SEQ ID NOs: 34-36, respectively;
(7) the selected Va CDRs 1, 2, 3 amino acid sequences are set forth in SEQ ID NOs: 37-39, respectively, and the selected Vb CDRs 1, 2, 3 amino acid sequences are set forth in SEQ ID NOs: 40-42, respectively;
(8) the selected Va CDRs 1, 2, 3 amino acid sequences are set forth in SEQ ID NOs: 43-45, respectively, and the selected Vb CDRs 1, 2, 3 amino acid sequences are set forth in SEQ ID NOs: 46-48, respectively;
(9) the selected Va CDRs 1, 2, 3 amino acid sequences are set forth in SEQ ID NOs: 49-51, respectively, and the selected Vb CDRs 1, 2, 3 amino acid sequences are set forth in SEQ ID NOs: 52-54, respectively;
(10) the selected Va CDRs 1, 2, 3 amino acid sequences are set forth in SEQ ID NOs: 61-63, respectively, and the selected Vb CDRs 1, 2, 3 amino acid sequences are set forth in SEQ ID NOs: 64-66, respectively;
(11) the selected Va CDRs 1, 2, 3 amino acid sequences are set forth in SEQ ID NOs: 67-69, respectively, and the selected Vb CDRs 1, 2, 3 amino acid sequences are set forth in SEQ ID NOs: 70-72, respectively;
(12) the selected Va CDRs 1, 2, 3 amino acid sequences are set forth in SEQ ID NOs: 73-75, respectively, and the selected Vb CDRs 1, 2, 3 amino acid sequences are set forth in SEQ ID NOs: 76-78, respectively;
(13) the selected Va CDRs 1, 2, 3 amino acid sequences are set forth in SEQ ID NOs: 79-81, respectively, and the selected Vb CDRs 1, 2, 3 amino acid sequences are set forth in SEQ ID NOs: 82-84, respectively; or
(14) the selected Va CDRs 1, 2, 3 amino acid sequences are set forth in SEQ ID NOs: 85-87, respectively, and the selected Vb CDRs 1, 2, 3 amino acid sequences are set forth in SEQ ID NOs: 88-90, respectively.

5. A T cell receptor (TCR) or antigen-binding fragment thereof, comprising
an alpha chain comprising a variable alpha (Va) region, wherein the Va region comprises a complementarity determining region 1 (CDR-1), a complementarity determining region 2 (CDR-2), and a complementarity determining region 3 (CDR-3), wherein the Va CDR1 region comprises an amino acid sequence that is at least 80% identical to SEQ ID NO: 55, the Va CDR2 region comprises an amino acid sequence that is at least 80% identical to SEQ ID NO: 56, the Va CDR3 region comprises an amino acid sequence that is at least 80% identical to SEQ ID NO: 57; and
a beta chain comprising a variable beta (Vb) region, wherein the Vb region comprises a complementarity determining region 1 (CDR-1), a complementarity determining region 2 (CDR-2), and a complementarity determining region 3 (CDR-3), wherein the Vb CDR1 region comprises an amino acid sequence that is at least 80% identical to SEQ ID NO: 58, the Vb CDR2 region comprises an amino acid sequence that is at least 80% identical to SEQ ID NO: 59, the Vb CDR3 region comprises an amino acid sequence that is at least 80% identical to SEQ ID NO: 60.

6. The TCR or antigen-binding fragment thereof of any one of claims 1-5, wherein the TCR or antigen-binding fragment thereof binds to or recognizes a peptide epitope of LMP2 (FLYALALLL) (SEQ ID NO: 139) that is presented by a major histocompatibility complex (MHC) molecule.

7. The TCR or antigen-binding fragment thereof of claim 6, wherein the MHC molecule is an HLA-A2 molecule.

8. The T cell receptor (TCR) or antigen-binding fragment thereof of claim 1, wherein the Va region is encoded by a sequence from rearrangement of a human TRAV gene segment and a human TRAJ gene segment, and the Vb region is encoded by a sequence from rearrangement of a human TRBV gene segment, optionally a human TRBD gene segment, and a human TRBJ gene segment, wherein
the TRAV gene segment is TRAV21;
the TRAJ gene segment is TRAJ33;
the TRBV gene segment is TRBV10-2;
the TRBD gene segment is TRBD1 or TRBD2; and
the TRBJ gene segment is TRBJ2-7.

9. The T cell receptor (TCR) or antigen-binding fragment thereof of claim 1 or 8,
wherein the Va CDR1 region comprises an amino acid sequence that is at least 80% identical to SEQ ID NO: 143, the Va CDR2 region comprises an amino acid sequence that is at least 80% identical to SEQ ID NO: 144, the Va CDR3 region comprises an amino acid sequence that is at least 80% identical to SEQ ID NO: 145; and
wherein the Vb CDR1 region comprises an amino acid sequence that is at least 80% identical to SEQ ID NO: 146, the Vb CDR2 region comprises an amino acid sequence that is at least 80% identical to SEQ ID NO: 147, the Vb CDR3 region comprises an amino acid sequence that is at least 80% identical to SEQ ID NO: 148.

10. A T cell receptor (TCR) or antigen-binding fragment thereof, comprising
an alpha chain comprising a variable alpha (Va) region, wherein the Va region comprises a complementarity determining region 1 (CDR-1), a complementarity determining region 2 (CDR-2), and a complementarity determining region 3 (CDR-3), wherein the Va CDR1 region comprises an amino acid sequence that is at least 80% identical to SEQ ID NO: 143, the Va CDR2 region comprises an amino acid sequence that is at least 80% identical to SEQ ID NO: 144, the Va CDR3 region comprises an amino acid sequence that is at least 80% identical to SEQ ID NO: 145; and
a beta chain comprising a variable beta (Vb) region, wherein the Vb region comprises a complementarity determining region 1 (CDR-1), a complementarity determining region 2 (CDR-2), and a complementarity determining region 3 (CDR-3), wherein the Vb CDR1 region comprises an amino acid sequence that is at least 80% identical to SEQ ID NO: 146, the Vb CDR2 region comprises an amino acid sequence that is at least 80% identical to SEQ ID NO: 147, the Vb CDR3 region comprises an amino acid sequence that is at least 80% identical to SEQ ID NO: 148.

11. The TCR or antigen-binding fragment thereof of any one of claims 1 and 8-10, wherein the TCR or antigen-binding fragment thereof binds to or recognizes a peptide epitope of LMP2 CLGGLLTMV (SEQ ID NO: 167) and/or SLGGLLTMV (SEQ ID NO: 168) that is presented by a major histocompatibility complex (MHC) molecule.

12. The TCR or antigen-binding fragment thereof of claim 11, wherein the MHC molecule is an HLA-A2 molecule.

13. A T cell receptor (TCR) or antigen-binding fragment thereof, comprising:
an alpha chain comprising a variable alpha (Va) region, wherein the Va region comprises a complementarity determining region 1 (CDR-1), a complementarity determining region 2 (CDR-2), and a complementarity determining region 3 (CDR-3), wherein the Va CDR1 region comprises an amino acid sequence that is at least 80% identical to a selected Va CDR1 amino acid sequence, the Va CDR2 region comprises an amino acid sequence that is at least 80% identical to a selected Va CDR2 amino acid sequence, the Va CDR3 region comprises an amino acid sequence that is at least 80% identical to a selected Va CDR3 amino acid sequence; and
a beta chain comprising a variable beta (Vb) region, wherein the Vb region comprises a complementarity determining region 1 (CDR-1), a complementarity determining region 2 (CDR-2), and a complementarity determining region 3 (CDR-3), wherein the Vb CDR1 region comprises an amino acid sequence that is at least 80% identical to a selected Vb CDR1 amino acid sequence, the Vb CDR2 region comprises an amino acid sequence that is at least 80% identical to a selected Vb CDR2 amino acid sequence, the Vb CDR3 region comprises an amino acid sequence that is at least 80% identical to a selected Vb CDR3 amino acid sequence;
wherein the selected Va CDRs 1, 2, and 3 and Vb CDRs 1, 2, and 3 amino acid sequences are one of the following:
(1) the selected Va CDRs 1, 2, 3 amino acid sequences are set forth in SEQ ID NOs: 91, 92, and 93, respectively, and the selected Vb CDRs 1, 2, 3 amino acid sequences are set forth in SEQ ID NOs: 94, 95, and 96, respectively;
(2) the selected Va CDRs 1, 2, 3 amino acid sequences are set forth in SEQ ID NOs: 97, 98, and 99, respectively, and the selected Vb CDRs 1, 2, 3 amino acid sequences are set forth in SEQ ID NOs: 100, 101, and 102, respectively;
(3) the selected Va CDRs 1, 2, 3 amino acid sequences are set forth in SEQ ID NOs: 103, 104, and 105, respectively, and the selected Vb CDRs 1, 2, 3 amino acid sequences are set forth in SEQ ID NOs: 106, 107, and 108, respectively;
(4) the selected Va CDRs 1, 2, 3 amino acid sequences are set forth in SEQ ID NOs: 149, 150, and 151, respectively, and the selected Vb CDRs 1, 2, 3 amino acid sequences are set forth in SEQ ID NOs: 152, 153, and 154, respectively; and
(5) the selected Va CDRs 1, 2, 3 amino acid sequences are set forth in SEQ ID NOs: 155, 156, and 157, respectively, and the selected Vb CDRs 1, 2, 3 amino acid sequences are set forth in SEQ ID NOs: 158, 159, and 160, respectively.

14. The T cell receptor (TCR) or antigen-binding fragment thereof of claim 13, wherein the Va region is encoded by a sequence from rearrangement of a human TRAV gene segment and a human TRAJ gene segment, and the Vb region is encoded by a sequence from rearrangement of a human TRBV gene segment, optionally a human TRBD gene segment, and a human TRBJ gene segment, wherein
the TRAV gene segment is TRAV4, TRAV25, TRAV22, or TRAV6;
the TRAJ gene segment is TRAJ23, TRAJ47, TRAJ29, TRAJ43, or TRAJ11;
the TRBV gene segment is TRBV12-4, TRBV12-3, TRBV11-2, TRBV4-1, or TRBV11-2;
the TRBD gene segment is TRBD2; and
the TRBJ gene segment is TRBJ1-1, TRBJ2-1, TRBJ2-5, or TRBJ2-7.

15. The TCR or antigen-binding fragment thereof of claim 13 or 14, wherein the TCR or antigen-binding fragment thereof binds to or recognizes a peptide epitope of LMP2 (SSCSSCPLSK) (SEQ ID NO: 140) that is presented by a major histocompatibility complex (MHC) molecule.

16. The TCR or antigen-binding fragment thereof of claim 15, wherein the MHC molecule is an HLA-A11 molecule.

17. A T cell receptor (TCR) or antigen-binding fragment thereof, comprising:
an alpha chain comprising a variable alpha (Va) region, wherein the Va region comprises a complementarity determining region 1 (CDR-1), a complementarity determining region 2 (CDR-2), and a complementarity determining region 3 (CDR-3), wherein the Va CDR1 region comprises an amino acid sequence that is at least 80% identical to a selected Va CDR1 amino acid sequence, the Va CDR2 region comprises an amino acid sequence that is at least 80% identical to a selected Va CDR2 amino acid sequence, the Va CDR3 region comprises an amino acid sequence that is at least 80% identical to a selected Va CDR3 amino acid sequence; and
a beta chain comprising a variable beta (Vb) region, wherein the Vb region comprises a complementarity determining region 1 (CDR-1), a complementarity determining region 2 (CDR-2), and a complementarity determining region 3 (CDR-3), wherein the Vb CDR1 region comprises an amino acid sequence that is at least 80% identical to a selected Vb CDR1 amino acid sequence, the Vb CDR2 region comprises an amino acid sequence that is at least 80% identical to a selected Vb CDR2 amino acid sequence, the Vb CDR3 region comprises an amino acid sequence that is at least 80% identical to a selected Vb CDR3 amino acid sequence;
wherein the selected Va CDRs 1, 2, and 3 and Vb CDRs 1, 2, and 3 amino acid sequences are one of the following:
(1) the selected Va CDRs 1, 2, 3 amino acid sequences are set forth in SEQ ID NOs: 109, 110, and 111, respectively, and the selected Vb CDRs 1, 2, 3 amino acid sequences are set forth in SEQ ID NOs: 112, 113, and 114, respectively; or
(2) the selected Va CDRs 1, 2, 3 amino acid sequences are set forth in SEQ ID NOs: 115, 116, and 117, respectively, and the selected Vb CDRs 1, 2, 3 amino acid sequences are set forth in SEQ ID NOs: 118, 119, and 120, respectively.

18. The T cell receptor (TCR) or antigen-binding fragment thereof of claim 17, wherein the Va region is encoded by a sequence from rearrangement of a human TRAV gene segment and a human TRAJ gene segment, and the Vb region is encoded by a sequence from rearrangement of a human TRBV gene segment, optionally a human TRBD gene segment, and a human TRBJ gene segment, wherein
the TRAV gene segment is TRAV12-1;
the TRAJ gene segment is TRAJ21;
the TRBV gene segment is TRBV20-1;
the TRBD gene segment is TRBD1; and
the TRBJ gene segment is TRBJ2-5 or TRBJ2-3.

19. The TCR or antigen-binding fragment thereof of claim 17 or 18, wherein the TCR or antigen-binding fragment thereof binds to or recognizes a peptide epitope of LMP2 (IYVLVMLVL) (SEQ ID NO: 141) that is presented by a major histocompatibility complex (MHC) molecule.

20. A T cell receptor (TCR) or antigen-binding fragment thereof, comprising:
an alpha chain comprising a variable alpha (Va) region, wherein the Va region comprises a complementarity determining region 1 (CDR-1), a complementarity determining region 2 (CDR-2), and a complementarity determining region 3 (CDR-3), wherein the Va CDR1 region comprises an amino acid sequence that is at least 80% identical to a selected Va CDR1 amino acid sequence, the Va CDR2 region comprises an amino acid sequence that is at least 80% identical to a selected Va CDR2 amino acid sequence, the Va CDR3 region comprises an amino acid sequence that is at least 80% identical to a selected Va CDR3 amino acid sequence; and
a beta chain comprising a variable beta (Vb) region, wherein the Vb region comprises a complementarity determining region 1 (CDR-1), a complementarity determining region 2 (CDR-2), and a complementarity determining region 3 (CDR-3), wherein the Vb CDR1 region comprises an amino acid sequence that is at least 80% identical to a selected Vb CDR1 amino acid sequence, the Vb CDR2 region comprises an amino acid sequence that is at least 80% identical to a selected Vb CDR2 amino acid sequence, the Vb CDR3 region comprises an amino acid sequence that is at least 80% identical to a selected Vb CDR3 amino acid sequence;
wherein the selected Va CDRs 1, 2, and 3 and Vb CDRs 1, 2, and 3 amino acid sequences are one of the following:
(1) the selected Va CDRs 1, 2, 3 amino acid sequences are set forth in SEQ ID NOs: 121, 122, and 123, respectively, and the selected Vb CDRs 1, 2, 3 amino acid sequences are set forth in SEQ ID NOs: 124, 125, and 126, respectively;
(2) the selected Va CDRs 1, 2, 3 amino acid sequences are set forth in SEQ ID NOs: 127, 128, and 129, respectively, and the selected Vb CDRs 1, 2, 3 amino acid sequences are set forth in SEQ ID NOs: 130, 131, and 132, respectively;
(3) the selected Va CDRs 1, 2, 3 amino acid sequences are set forth in SEQ ID NOs: 133, 134, and 135, respectively, and the selected Vb CDRs 1, 2, 3 amino acid sequences are set forth in SEQ ID NOs: 136, 137, and 138, respectively; or
(4) the selected Va CDRs 1, 2, 3 amino acid sequences are set forth in SEQ ID NOs: 161, 162, and 163, respectively, and the selected Vb CDRs 1, 2, 3 amino acid sequences are set forth in SEQ ID NOs: 164, 165, and 166, respectively.

21. The T cell receptor (TCR) or antigen-binding fragment thereof of claim 15, wherein the Va region is encoded by a sequence from rearrangement of a TCR alpha variable (TRAV) gene segment and a TCR alpha joining (TRAJ) gene segment, wherein the Vb region is encoded by a sequence from rearrangement of a TCR beta variable (TRBV) gene segment, optionally a TCR beta diversity (TRBD) gene segment, and a TCR beta joining (TRBJ) gene segment, wherein
the TRAV gene segment is TRAV25, TRAV12-3, or TRAV21;
the TRAJ gene segment is TRAJ16, TRAJ3, or TRAJ35;
the TRBV gene segment is TRBV6-6, TRBV24-1, or TRBV30;
the TRBD gene segment is TRBD1 or TRBD2; and
the TRBJ gene segment is TRBJ1-1, TRBJ2-5, TRBJ2-3, or TRBJ2-7.

22. The TCR or antigen-binding fragment thereof of claim 20 or 21, wherein the TCR or antigen-binding fragment thereof binds to or recognizes a peptide epitope of LMP2 (TYGPVFMCL) (SEQ ID NO: 142) that is presented by a major histocompatibility complex (MHC) molecule.

23. The TCR or antigen-binding fragment thereof of claim 19 or 22, wherein the MHC molecule is an HLA-A24 molecule.

24. The TCR or antigen-binding fragment thereof of any one of claims 1-23, wherein the alpha chain comprises a mouse alpha chain constant region, and the beta chain comprises a mouse beta chain constant region.

25. The TCR or antigen-binding fragment thereof of any one of claims 1-23, wherein the alpha chain comprises a human alpha chain constant region, and the beta chain comprises a human beta chain constant region.

26. The TCR or antigen-binding fragment thereof of any of claims 1-25, wherein, the TCR or antigen-binding fragment thereof, when expressed on the surface of a T cell, stimulates cytotoxic activity against a target cancer cell.

27. The TCR or antigen-binding fragment thereof of claim 26, wherein the target cancer cell comprises a nucleic acid sequence encoding LMP2 or expresses LMP2.

28. A vector comprising a nucleic acid encoding TCR or antigen-binding fragment thereof of any one of claims 1-27.

29. A vector comprising:
a) a first nucleic acid sequence encoding a TCR alpha chain comprising an alpha chain variable region of a human anti-LMP2 TCR and an alpha chain constant region; and
b) a second nucleic acid sequence encoding a TCR beta chain comprising a beta chain variable region of the human anti-LMP2 TCR and a beta chain constant region,
wherein the TCR alpha chain and the TCR beta chain form the TCR or antigen-binding fragment thereof of any one of claims 1-27.

30. The vector of claim 29, wherein the first nucleic acid sequence and the second nucleic acid sequence is linked by a linker sequence.

31. The vector of claim 30, wherein the linker sequence is a P2A sequence.

32. The vector of any one of claims 28-31, wherein the vector is an expression vector, a viral vector, a retroviral vector, or a lentiviral vector.

33. The vector of claim 32, wherein the retroviral vector is pMP71.

34. An engineered cell comprising the vector of any one of claims 28-33.

35. An engineered cell, comprising the TCR or antigen-binding fragment thereof of any of claims 1-27.

36. The engineered cell of claim 34 or 35, wherein the TCR or antigen binding fragment thereof is heterologous to the cell.

37. The engineered cell of any one of claims 34-36, wherein the engineered cell is a cell line.

38. The engineered cell of any one of claims 34-36, wherein the engineered cell is a primary cell obtained from a subject (e.g., a human subject).

39. The engineered cell of any of claims 34-38, wherein the engineered cell is a T cell.

40. The engineered cell of claim 39, wherein the T-cell is isolated from a human subject.

41. The engineered cell of claim 39, wherein the T cell is CD8+.

42. The engineered cell of claim 39, wherein the T cell is CD4+.

43. The engineered cell of any one of claims 34-42, wherein the engineered cell expresses a bifunctional trap protein.

44. The engineered cell of claim 43, wherein the bifunctional trap protein targets a checkpoint inhibitor (e.g., PD-1) and a member of the transforming growth factor beta family (e.g., TGF-β).

45. The engineered cell of any one of claims 34-42, wherein the engineered cell expresses an antibody or antigen-binding fragment thereof targeting a checkpoint inhibitor (e.g., PD-1).

46. The engineered cell of any one of claims 34-42 and 45, wherein the engineered cell expresses a protein that binds to a member of the transforming growth factor beta family (e.g., TGF-β).

47. A method for producing the engineered cell, comprising introducing the vector of any one of claims 28-33 into the cell in vitro or ex vivo.

48. The method of claim 47, wherein the introducing step is carried out by transduction.

49. A method of treating a disease or a disorder, comprising administering the engineered cell of any one of claims 34-46 to a subject having a disease or disorder associated with EBV.

50. The method of claim 49, wherein the disease or disorder associated with EBV is a cancer.

51. A method of treating a tumor in a subject, the method comprising
administering to the subject in need thereof an engineered T cell, comprising a nucleic acid encoding the TCR or antigen-binding fragment thereof of claims 1-27 that specifically binds to an antigen in the tumor.

52. A method of treating a tumor in a subject, the method comprising
administering to the subject in need thereof
(a) an engineered T cell, comprising: a nucleic acid encoding the TCR or antigen-binding fragment thereof of claims 1-27 that specifically binds to an antigen in a tumor; and
(b) either one or both of a checkpoint inhibitor and a protein that binds to a member of the transforming growth factor beta family (e.g., TGF-β).

53. A method of treating a tumor in a subject, the method comprising
administering to the subject in need thereof
an engineered T cell, comprising: a nucleic acid encoding
(a) the TCR or antigen-binding fragment thereof of claims 1-27 that specifically binds to an antigen in a tumor; and
(b) a bifunctional trap protein that targets a checkpoint inhibitor and a member of the transforming growth factor beta family (e.g., TGF-β).

54. The method of any one of claims 51-53, wherein the tumor is an EBV-associated tumor.

55. The method of claim 50 or 54, wherein the cancer or the EBV-associated tumor is Burkitt’s lymphoma, immunosuppressive lymphoma, diffuse large B-cell lymphoma, diffuse large B-cell lymphoma associated with chronic inflammation, lymphomatoid granulomatosis, plasmablastic lymphoma, primary effusion lymphoma, post-transplant lymphoproliferative disorder, nasopharyngeal carcinoma, gastric adenocarcinoma, lymphoepithelioma-associated carcinoma, immunodeficiency-related leiomyosarcoma, or Hodgkin’s lymphoma.
